SEQUENCE LISTING (103 sequences)

SEQ ID NO: 1 - DNA, 20 bp, Artificial Sequence
  Synthetic
aagtccagcaatcagagcta

SEQ ID NO: 2 - DNA, 20 bp, Artificial Sequence
  Synthetic
tgtcttacaggccctgcctc

SEQ ID NO: 3 - DNA, 181 bp, Artificial Sequence
  Synthetic
actcgggcctcactcactgggaactcgatggattctggggcacagttatccttcagcagattctccttcaggtcacagcgaggtgagccgggtggcagggcctgtaagacaggagcccaaagagaagtccagcaatcagagctatgccgactctctacctcctgcaggccctaccacttcc

SEQ ID NO: 4 - DNA, 30 bp, Artificial Sequence
  Synthetic PCR primer
cgtggaattcgctggtctaccaggcatctt

SEQ ID NO: 5 - DNA, 29 bp, Artificial Sequence
  Synthetic PCR primer
ccgaagcttaccttgtgctctatgcccac

SEQ ID NO: 6 - DNA, 30 bp, Artificial Sequence
  Synthetic PCR primer
cgtggaattcggcatcttactgtacaggct

SEQ ID NO: 7 - DNA, 29 bp, Artificial Sequence
  Synthetic PCR primer
ggcaagcttaagacttcctcctcagacct

SEQ ID NO: 8 - DNA, 87 bp, Homo sapiens
ggaggtagagagtcgccatagctctgattgctggacttctctttgggctcctgtcttacaggccctgcctctgggctcacctcgctg

SEQ ID NO: 9 - DNA, 87 bp, Homo sapiens
cctccatctctcagcggtatcgagactaacgacctgaagagaaacccgaggacagaatgtccgggacggagacccgagtggagcgac

SEQ ID NO: 10 - DNA, 21 bp, Homo sapiens
gccctgcctctgggctcacct

SEQ ID NO: 11 - DNA, 21 bp, Homo sapiens
gccctgcctccgggctcacct

SEQ ID NO: 12 - DNA, 18 bp, Homo sapiens
ggccctgccacccggctc

SEQ ID NO: 13 - DNA, 42 bp, Homo sapiens
agcttcagttctgcaatcagagctaaagattagccctgctgc

SEQ ID NO: 14 - DNA, 41 bp, Homo sapiens
tcctcaggttcagtaatcagagctatggaggctaccataaa

SEQ ID NO: 15 - DNA, 41 bp, Homo sapiens
atgaccaggccagcaaccagagctatgggtggggctgctat

SEQ ID NO: 16 - DNA, 42 bp, Homo sapiens
agtcaaataccaacgatcagagctacagacactaagttttta

SEQ ID NO: 17 - DNA, 42 bp, Homo sapiens
catgtaaatacagcaatcagagcaagagagcagggtgttgac

SEQ ID NO: 18 - DNA, 42 bp, Homo sapiens
gtctctctcgtacatgccctgcctcaggcttaaaattgacac

SEQ ID NO: 19 - DNA, 43 bp, Homo sapiens
agctgtgtctttcagcccctgcctcaggcaaccatgctccttt

SEQ ID NO: 20 - DNA, 42 bp, Homo sapiens
tcccaggtttttcaggccctgcctccagacacagaattgcct

SEQ ID NO: 21 - DNA, 41 bp, Homo sapiens
tgagaggttttccaggccctgcctcaagccaagaggcaaga

SEQ ID NO: 22 - DNA, 42 bp, Homo sapiens
tgttgtgtcctggaggccctgcctcaggtttcccagtttggc

SEQ ID NO: 23 - DNA, 23 bp, Homo sapiens
aagtccagcaatcagagctatgg

SEQ ID NO: 24 - DNA, 23 bp, Homo sapiens
cagttctgcaatcagagctaaag

SEQ ID NO: 25 - DNA, 20 bp, Artificial Sequence
  Synthetic
gagaaccatcaacccaatgc

SEQ ID NO: 26 - DNA, 20 bp, Artificial Sequence
  Synthetic
gtgtatggcagtttgtccac

SEQ ID NO: 27 - DNA, 23 bp, Homo sapiens
aggttcagtaatcagagctatgg

SEQ ID NO: 28 - DNA, 21 bp, Artificial Sequence
  Synthetic PCR primer
gccataaagtgggatcatttg

SEQ ID NO: 29 - DNA, 20 bp, Artificial Sequence
  Synthetic PCR primer
accataggtctcctcgatgt

SEQ ID NO: 30 - DNA, 23 bp, Homo sapiens
caggccagcaaccagagctatgg

SEQ ID NO: 31 - DNA, 20 bp, Artificial Sequence
  Synthetic PCR primer
tcaatgctaaacaacggcac

SEQ ID NO: 32 - DNA, 20 bp, Artificial Sequence
  Synthetic PCR primer
gctgctgatctgaaagggta

SEQ ID NO: 33 - DNA, 23 bp, Homo sapiens
aataccaacgatcagagctacag

SEQ ID NO: 34 - DNA, 20 bp, Artificial Sequence
  Synthetic PCR primer
caactgagcctttcctacca

SEQ ID NO: 35 - DNA, 20 bp, Artificial Sequence
  Synthetic PCR primer
agagacggaattgagacacc

SEQ ID NO: 36 - DNA, 23 bp, Homo sapiens
aaatacagcaatcagagcaagag

SEQ ID NO: 37 - DNA, 20 bp, Artificial Sequence
  Synthetic PCR primer
attaaagcttctgctggcga

SEQ ID NO: 38 - DNA, 21 bp, Artificial Sequence
  Synthetic PCR primer
tgatactccacctgacaactc

SEQ ID NO: 39 - DNA, 23 bp, Homo sapiens
tgtcttacaggccctgcctctgg

SEQ ID NO: 40 - DNA, 23 bp, Homo sapiens
tctcgtacatgccctgcctcagg

SEQ ID NO: 41 - DNA, 20 bp, Artificial Sequence
  Synthetic PCR primer
ctgtctctgctgcacatcta

SEQ ID NO: 42 - DNA, 20 bp, Artificial Sequence
  Synthetic PCR primer
gttcaggtggagacaggaat

SEQ ID NO: 43 - DNA, 23 bp, Homo sapiens
tgtctttcagcccctgcctcagg

SEQ ID NO: 44 - DNA, 23 bp, Artificial Sequence
  Synthetic PCR primer
taagaccatatggacatggcaga

SEQ ID NO: 45 - DNA, 21 bp, Artificial Sequence
  Synthetic PCR primer
gagtagcaaatggctggaaca

SEQ ID NO: 46 - DNA, 23 bp, Homo sapiens
ggtttttcaggccctgcctccag

SEQ ID NO: 47 - DNA, 20 bp, Artificial Sequence
  Synthetic PCR primer
gagtttgctgtgcagagatg

SEQ ID NO: 48 - DNA, 20 bp, Artificial Sequence
  Synthetic PCR primer
tgcttaagtgtgggccttta

SEQ ID NO: 49 - DNA, 23 bp, Homo sapiens
ggttttccaggccctgcctcaag

SEQ ID NO: 50 - DNA, 20 bp, Artificial Sequence
  Synthetic PCR primer
aaagagagatgagctcgtgg

SEQ ID NO: 51 - DNA, 20 bp, Artificial Sequence
  Synthetic PCR primer
tttcaagagtggagtggctt

SEQ ID NO: 52 - DNA, 23 bp, Homo sapiens
tgtcctggaggccctgcctcagg

SEQ ID NO: 53 - DNA, 20 bp, Artificial Sequence
  Synthetic PCR primer
gaaattctttggccacctcc

SEQ ID NO: 54 - DNA, 20 bp, Artificial Sequence
  Synthetic PCR primer
cacagtgccaaatatcagca

SEQ ID NO: 55 - DNA, 30 bp, Artificial Sequence
  synthetic - primer
cgtggaattcaagtggagcacacctatgag

SEQ ID NO: 56 - DNA, 29 bp, Artificial Sequence
  synthetic primer
ggcaagcttaccttgctttggcattgttt

SEQ ID NO: 57 - DNA, 1599 bp, Artificial Sequence
  synthetic
ggcgtcgaattcagcacttcaagtgaacatggaggagccagaatccaaacagcaagattgtgctgctggacgtgccggtccgggcagaggcccaagtggagctgcgagggtgagaggccaggggtggagaagggagatggcattcagggctctaaactccagggggcgctggggaaacctcacaggccaatcagggcatcacactctctctgggggtcttgggcacctgcaggaactcctttccagcctccctggtggtggcagcagaagaaggtgagagggagcagaacagcttggacagctggggacccaaagtggagcacacctatgaggtattggggagcctcgcgtccctggctggggtgagcgggtcctcagaactccgggtgaggcgctaagctccccacaccctgccaccaccaccccttcagctccacaacaatggccctgggactgtgaatggtcttcacctcagcatccaccttccgggacagtcccagccctccgacctgctctacatcctggatatacagccccaggggggccttcagtgcttcccacagcctcctgtcaaccctctcaaggtaagagctgggtggaagaaagacctgggaaggcggccccagaccaaccaacgttgcacctctgtgggctggggttcgggggagacctgggcctgaccactcctttgcccccccaggtggactgggggctgcccagccccagcccctcccccattcacccggcccatcacaagcgggatcgcagacagatcttcctgccagagcccgagcagccctcgaggcttcaggatccagttctcgtagtgagcaggctctctggtctctggcccggcctccccgggacccacggggcagaggggatgggaggagggagaggggtccgggtgtgctgtgggcctctgtgggccacgcttggtccctgggagcacttcaattgcagttggagtagcatgctggcttgtgtctggggtgagctgaaagacacttgcactttttaaaagcttcccagtacgttaaggagcataaaacaatgccaaagcaaggttatcatagatctgagcattgtgcgctgggggatgaccctccctgcatctctgggactatgtgagcaagcccgtggaaagacagcatccgaagcttggatccaaggcccttcctgatgggaaggccaccgcttcctgaacccccggccccttctgcgttgggtcctgggggtaagggggtgggggatgatggggtgatgggccgggacgggctggggactgacgatgcttcccctcagagctgcgactcggcgccctgtactgtggtgcagtgtgacctgcaggagatggcgcgcgggcagcgggccatggtcacggtgctggccttcctgtggctgcccagcctctaccaggtggggtgggccgtggtggggcggggccgggccttctgggcggggaccactttgctctgggaggggcggggtttggtgtgggagggcaggaagagagggaaggcaaggtttactttgggggattgcagtgggattaggtcagaggcctccatgttcacttgaagtgctgaattcgcagcg

SEQ ID NO: 58 - DNA, 20 bp, Artificial Sequence
  synthetic - guide RNA
gagtagcgcgagcacagcta

SEQ ID NO: 59 - DNA, 20 bp, Artificial Sequence
  synthetic - guide RNA
cggccccagaccaaccaccg

SEQ ID NO: 60 - DNA, 20 bp, Artificial Sequence
  synthetic - guide RNA
agcacttcaagtgaacatgg

SEQ ID NO: 61 - DNA, 20 bp, Artificial Sequence
  synthetic - guide RNA
gggcagcccccagtccacct

SEQ ID NO: 62 - DNA, 199 bp, Artificial Sequence
  synthetic
ctcgagggctgctcgggctctggcaggaagatctgtctgcgatcccgcttgtgatgggccgggtgaatgggagaggggctggggctgggcagcccccagtccatctgcaggggcaaaggagtggtcaggcccaggtctcccccgaaccccagcccacagaggtgccccggtggttggtctggggccgccttcccaggtc

SEQ ID NO: 63 - DNA, 25 bp, Artificial Sequence
  synthetic
caccgatcagcattaagtaccactg

SEQ ID NO: 64 - DNA, 25 bp, Artificial Sequence
  synthetic
aaaccagtggtacttaatgctgatc

SEQ ID NO: 65 - DNA, 25 bp, Artificial Sequence
  synthetic
caccgagtgtgcattgccacctcag

SEQ ID NO: 66 - DNA, 25 bp, Artificial Sequence
  synthetic
aaacctgaggtggcaatgcacactc

SEQ ID NO: 67 - DNA, 25 bp, Artificial Sequence
  synthetic
caccgagcattaagtaccactgagg

SEQ ID NO: 68 - DNA, 25 bp, Artificial Sequence
  synthetic
aaaccctcagtggtacttaatgctc

SEQ ID NO: 69 - DNA, 25 bp, Artificial Sequence
  synthetic
caccggtccatcgtttcactgtacc

SEQ ID NO: 70 - DNA, 25 bp, Artificial Sequence
  synthetic
aaacggtacagtgaaatgatggacc

SEQ ID NO: 71 - DNA, 25 bp, Artificial Sequence
  synthetic
caccggcccatcatttctctgaacc

SEQ ID NO: 72 - DNA, 25 bp, Artificial Sequence
  synthetic
aaacggttcagagaaacgatgggcc

SEQ ID NO: 73 - DNA, 25 bp, Artificial Sequence
  synthetic
caccgataaaagtgcatgttagagg

SEQ ID NO: 74 - DNA, 25 bp, Artificial Sequence
  synthetic
aaaccctctaacatgcacttttatc

SEQ ID NO: 75 - DNA, 25 bp, Artificial Sequence
  synthetic
caccggaatatttttcttactccta

SEQ ID NO: 76 - DNA, 25 bp, Artificial Sequence
  synthetic
aaactaggagtaagaaaaatattcc

SEQ ID NO: 77 - DNA, 25 bp, Artificial Sequence
  synthetic
caccgatgtcagtatcaccaagaag

SEQ ID NO: 78 - DNA, 25 bp, Artificial Sequence
  synthetic
aaaccttcttggtgatactgacatc

SEQ ID NO: 79 - DNA, 25 bp, Artificial Sequence
  synthetic
caccgaacaagacatgtgagtgaag

SEQ ID NO: 80 - DNA, 25 bp, Artificial Sequence
  synthetic
aaaccttcactcacatgtcttgttc

SEQ ID NO: 81 - DNA, 25 bp, Artificial Sequence
  synthetic
caccgtaagtatttcaacaatgaga

SEQ ID NO: 82 - DNA, 25 bp, Artificial Sequence
  synthetic
aaactctcattgttgaaatacttac

SEQ ID NO: 83 - DNA, 25 bp, Artificial Sequence
  synthetic
caccgcagtggggcactattgtaca

SEQ ID NO: 84 - DNA, 25 bp, Artificial Sequence
  synthetic
aaactgtacaatagtgccccactgc

SEQ ID NO: 85 - DNA, 25 bp, Artificial Sequence
  synthetic
caccgcctgtacaatagtgccccac

SEQ ID NO: 86 - DNA, 25 bp, Artificial Sequence
  synthetic
aaacgtggggcactattgtacaggc

SEQ ID NO: 87 - DNA, 25 bp, Artificial Sequence
  synthetic
caccgcaacctactcgttgctgctt

SEQ ID NO: 88 - DNA, 25 bp, Artificial Sequence
  synthetic
aaacaagcagcaacgagtaggttgc

SEQ ID NO: 89 - DNA, 24 bp, Artificial Sequence
  synthetic
caccgatatcttttggagctgtcc

SEQ ID NO: 90 - DNA, 24 bp, Artificial Sequence
  synthetic
aaacggacagctccaaaagatatc

SEQ ID NO: 91 - DNA, 25 bp, Artificial Sequence
  synthetic
caccgcactaaccaggtattcattg

SEQ ID NO: 92 - DNA, 25 bp, Artificial Sequence
  synthetic
aaaccaatgaatacctggttagtgc

SEQ ID NO: 93 - DNA, 24 bp, Artificial Sequence
  synthetic
caccgggtggggctcgtttttccc

SEQ ID NO: 94 - DNA, 24 bp, Artificial Sequence
  synthetic
aaacgggaaaaacgagccccaccc

SEQ ID NO: 95 - DNA, 87 bp, Homo sapiens
gggcattcctgaagctgacagcattcgggccgagatgtctcgctccgtggccttagctgtgctcgcgctactctctctttctggcct

SEQ ID NO: 96 - DNA, 87 bp, Homo sapiens
aggccagaaagagagagtagcgcgagcacagctaaggccacggagcgagacatctcggcccgaatgctgtcagcttcaggaatgccc

SEQ ID NO: 97 - DNA, 24 bp, Homo sapiens
tgggggctgcccagccccagcccc

SEQ ID NO: 98 - DNA, 27 bp, Artificial Sequence
  synthetic
gacgtctacctgacccccgacgggtcg

SEQ ID NO: 99 - DNA, 87 bp, Homo sapiens
cctgggcctgaccactcctttgcccccccaggtggactgggggctgcccagccccagcccctcccccattcacccggcccatcacaa

SEQ ID NO: 100 - DNA, 87 bp, Homo sapiens
ttgtgatgggccgggtgaatgggggaggggctggggctgggcagcccccagtccacctgggggggcaaaggagtggtcaggcccagg

SEQ ID NO: 101 - DNA, 23 bp, Homo sapiens
ccctgcagatggactggggctgc

SEQ ID NO: 102 - protein, 5 aa, Homo sapiens
SSTTG

SEQ ID NO: 103 - protein, 5 aa, Homo sapiens
LSTTQ

(19) United States
(12) Patent Application Publication
(10) Pub. No.: US 2020/0332259 A1
(43) Pub. Date: Oct. 22, 2020

(54) METHOD TO BIOENGINEER DESIGNER RED BLOOD CELLS USING GENE EDITING AND STEM CELL METHODOLOGIES

(71) Applicant: VERSITI BLOOD RESEARCH INSTITUTE FOUNDATION, INC., Milwaukee, WI (US)

(72) Inventors: PETER JAY NEWMAN, Bayside, WI (US); Sridhar Rao, Brookfield, WI (US); Nanyan Zhang, Wauwatosa, WI (US); Huiying Zhi, Brookfield, WI (US)

(21) Appl. No.: 16/913,741

(22) Filed: Jun. 26, 2020

Related U.S. Application Data
(63) Continuation-in-part of application No. 14/931,321, filed on Nov. 3, 2015, now Pat. No. 10,725,041.
(60) Provisional application No. 62/074,870, filed on Nov. 4, 2014.

(51) Int. Cl. C12N 5/0789; C12N 9/22; C12N 15/113; C12N 5/078

(52) U.S. Cl. CPC C12N 5/0647; C12N 9/22; C12N 15/113; C12N 2506/45; C12N 2310/20; C12N 2800/80; C12N 5/0644

(57) ABSTRACT

A method of creating cells expressing specific red blood cell antigens is disclosed. In one embodiment, the method comprises the steps of (a) combining one or more guide RNAs targeting within a red blood cell antigen target locus; (b) adding a repair template comprising a mutation in the target locus flanked by a homology arm on each side, wherein the template may additionally include a diagnostic restriction enzyme site at the target locus; (c) ligating the guide sequence of step (b) into a plasmid which also expresses a nuclease and, optionally, a selectable marker or a reporter gene; (d) transfecting pluripotent cells with the plasmid of step (c) in the presence of an HDR repair oligo; (e) cloning and testing the resulting reporter positive clones for expression of the antigen target of interest; and (f) culturing the resulting cells to expand their numbers or to create a differentiated cell type of interest.

### CROSS-REFERENCE TO RELATED APPLICATIONS

This application is a continuation-in-part of U.S. application Ser. No. 14/931,321, filed Nov. 3, 2015, which claims the benefit of U.S. Provisional Patent Application Ser. No. 62/074,870 filed Nov. 4, 2014, each of which are incorporated herein by reference in their entirety.

### STATEMENT REGARDING FEDERALLY SPONSORED RESEARCH

This invention was made with government support under Grant No. P01-HL44612 awarded by the National Institutes of Health. The U.S. Government has certain rights in this invention.

### REFERENCE TO A SEQUENCE LISTING SUBMITTED VIA EFS-WEB

The content of the ASCII text file of the sequence listing named “160180_00145_ST25.txt” which is 20.6 kb in size was created on Jun. 26, 2020 and electronically submitted via EFS-Web herewith the application is incorporated herein by reference in its entirety.

### BACKGROUND

Platelet alloantigens are substances that induce the production of alloantibodies when platelets bearing such antigens are infused into patients who lack the specific alloantigen Immune responses to platelet alloantigens are involved in the pathogenesis of several clinical syndromes including neonatal alloimmune thrombocytopenia, post-transfusion purpura, and refractory responses to platelet transfusion. In addition, immune thrombocytopenia can be an unusual complication of a type of graft-versus-host disease in which donor lymphocytes make alloantibodies specific for the platelets produced by the recipient of an organ allograft.

Patients can lack a particular platelet-associated antigen altogether because they have defective alleles of the gene encoding the antigen. Such patients can make antibodies against platelets of virtually all donors that bear the platelet-associated antigen. For example, patients with Bernard-Soulier syndrome, who lack platelet GPIb-V-IX, or patients with Glanzmann thrombasthenia, who lack expression of GPIIb (CD41) and GPIIIa (CD61), can be induced to make broadly-reactive antiplatelet antibodies. Also, several percent of Japanese and approximately 0.3 percent of Caucasians are deficient in CD36, one of the major platelet glycoproteins of platelets that also is known as GPIV. Because these patients lack a platelet antigen, they can develop antiplatelet antibodies specific for the deficient platelet protein after receiving transfusions of platelets from normal donors or after pregnancy. More commonly, platelet-specific alloantigens result from genetic polymorphism in genes encoding functional platelet proteins. These alloantigens first were defined by antiplatelet antibodies discovered in the sera of multiparous females who gave birth to infants with neonatal thrombocytopenia. Many of these subsequently were found to recognize allotypic determinants of platelet-associated membrane glycoproteins, such as GPIIb/IIIa (CD41/CD61). Each of these allotypic determinants may be generated by only a single amino acid substitution in a major platelet-associated glycoprotein. However, it is possible that glycosylation may contribute to or influence the expression of certain Human Platelet Alloantigenic (HPA) epitopes, such as those associated with human platelet antigen 3 (HPA-3). In any case, these amino acid substitutions generally do not appear to affect the function of platelets in vitro. However, it is conceivable that the genetic polymorphism in platelet glycoproteins may be associated with more subtle differences in platelet physiology that can contribute to the relative risk for thrombosis and/or atherosclerosis. (Williams Hematology, Chapter 138)

The human leukocyte histocompatibility antigens, HLA, are polymorphic cell surface glycoproteins that present antigen peptide fragments to T-cell receptors. HLA antigens are encoded by multiple, closely linked genes, located in a 4-Mb region of DNA on chromosome 6, that comprise the major histocompatibility complex (MHC) and play a central role in the regulation of immune responses. In general, the MHC genes are inherited as a single unit in simple Mendelian fashion. The products of the MHC HLA-A, HLA-B, and HLA-C genes are called class I antigens. Class I antigens are expressed on essentially all tissues in the body and present small peptide fragments to CD8+ T cells. (Williams Hematology, Chapter 138)

There are six major groups of HLA antigens: HLA-A, HLA-B, HLA-C, HLA-DR, HLA-DQ, and HLA-DP. These groups are divided into classes of antigens designated as class I and class II, representing the two types of HLA molecules. The HLA-A, HLA-B, and HLA-C antigens are the class I antigens. The HLA-DR, HLA-DQ, and HLA-DP antigens are the class II antigens. (Williams Hematology, Chapter 138)

In addition to the HLA antigens, platelets also express glycoproteins that can be recognized by autoantibodies or by antibodies made by recipients of platelet transfusions. The latter are due to platelet alloantigens that reflect polymorphism in the genes encoding major platelet glycoproteins. Immune responses to platelet alloantigens are involved in the pathogenesis of several clinical syndromes, including neonatal alloimmune thrombocytopenia, post-transfusion purpura, and refractory responses to platelet transfusion. (Williams Hematology, Chapter 138)

The inventors have discovered a method of creating human platelets expressing specific HPA isotypes utilizing CRISPR/Cas9 gene editing methods and laboratory cell culture techniques. Deletion of the β₂ microglobulin gene offers distinct practical advantages that will be outlined in the description of the invention.

The inventors have discovered a method to generate human platelets that express any minor or major HPA that is desired, so called “designer platelets”. After demonstrating that one can convert PI^{A1} to PI^{A2} in DAMI cells, the inventors have most recently shown this conversion in human induced pluripotent stem (iPS) cells which can be differentiated into megakaryocytes and then platelets using methods known in the art. Our initial anticipated use will be the development of a new platform for rapid flow cytometric detection of rare platelet antigens. This will be made useful and easier than antigen capture ELISA test (ACE) or modified antigen capture ELISA test (MACE) because we will also knock out β₂ microglobulin in the iPS cells so that anti-HLA antibodies in maternal or patient sera will have no Class I targets to bind to, hopefully simplifying the assay and lowering back-ground.

In concept, it would be very useful to have such a panel for laboratory testing. Even though the market might be small, the project may provide proof-of principal for future studies to express rare RBC antigens (of which there are many). Right now, reference blood banks maintain frozen RBC panels expressing various low frequency RBC antigens or (equally important) lacking high frequency (public) antigens and they thaw them out when they need to check specificity of an unknown antibody in a patient. Producing “designer RBCs”, that look identical to physiologic RBC's, could be a serious technical challenge because the cultured cells need to shed their nucleus, among other things, and techniques to do this are not currently completely finalized. However, one could express the rare RBC antigens in nucleated RBC's, anucleated RBC's, platelets, iPS cells, or iPS cell and then use these cell types as laboratory controls and sources of these rare antigens.

An additional use of iPS-derived designer platelets will be to provide rare platelet types for transfusion. This will require the use of a platelet bioreactor. The commercial use of platelet bioreactors is not yet commonplace. However, one advantage of this strategy, is the gene editing arm of the technology, which allows you to make platelets of specific HPA types. The therapeutic use of platelets that lack specific HLA antigens or express matching HLA antigens could be a solution to various forms of platelet refractoriness. The platelets would be group ABO negative or group 0, to rule out issues with ABO compatibility. HPA-1a-negative platelets might be useful for the most common form of NAIT. Platelets matched for other HPA antigens are occasionally useful in immunized thrombocytopenic patients.

### BRIEF SUMMARY OF THE INVENTION

In a some aspects, provided herein is a method of creating cells expressing specific platelet or red blood cell alloantigens by combining gene editing techniques and cell culture techniques employing pluripotent cells, the method comprising the steps of editing a plutipotent cell so that the cell expresses the alloantigen of interest and culturing the cell to expand or create a differentiated cell type. In a preferred embodiment, the cells are further edited by removal of HLA class I antigens.

The method comprises the steps of (a) combining one or more guide RNAs targeting within a platelet alloantigen target locus; (b) adding a repair template comprising a mutation in the target locus flanked by a homology arm on each side, wherein the template may additionally include a diagnostic restriction enzyme site at the target locus; (c) ligating the guide sequence of step (b) into a plasmid which also expresses a nuclease and, optionally, a selectable marker or a reporter gene; (d) transfecting pluripotent cells with the plasmid of step (c) in the presence of an HDR repair oligo; (e) cloning and testing the resulting reporter positive clones for expression of the alloantigen target of interest; and (f) culturing the resulting cells to expand their numbers or to create a differentiated cell type of interest.

In some embodiments, the method comprises the steps of (a) combining one or more guide RNAs targeting within a red cell alloantigen target locus; (b) adding a repair template comprising a mutation in the target locus flanked by a homology arm on each side, wherein the template may additionally include a diagnostic restriction enzyme site at the target locus; (c) ligating the guide sequences of step (b) into a plasmid which also expresses a nuclease and, optionally a selectable marker or a reporter gene; (d) transfecting pluripotent cells with the plasmid of step (c) in the presence of an HDR repair oligo; (e) cloning and testing the resulting reporter positive clones for expression of the alloantigen target of interest; and (f) culturing the resulting cells to expand their numbers or to create a differentiated cell type of interest.

In some embodiments, the method includes the step of further editing the cells in step (a) by removal of HLA class I antigens, preferably by genetic removal of the β₂ microglobulin gene.

In some aspects, provided herein is a method for creating a mammalian hematopoietic progenitor cell that does not express any Rh red blood cell antigen, the method comprising the steps of: a) providing one or more guide RNAs designed to target a gene selected from the group consisting of RHD, RHCE, and RHAG; b) ligating the guide RNA of step (a) into a plasmid encoding a Cas9 nuclease; c) transfecting mammalian induced pluripotent stem cells with the plasmid of step (b); d) cloning and selecting the resulting clones that do not express the Rh antigens; and e) differentiating the selected clones into mammalian hematopoietic progenitor cells that do not express the Rh antigens. In some embodiments, the target gene is RHD or RHCE. In some embodiments, the induced pluripotent stem cell comprising the RHD, RHCE, and RNAG genes. In some embodiments, the mammalian induced pluripotent stem cell is transfected with the plasmid of step (b) in the presence of a homology-directed repair (HDR) template oligonucleotide. In some embodiments, the HDR template oligonucleotide encodes a stop codon to be introduced into the target gene. In some embodiments, the HDR template oligonucleotide encodes missense mutation in the target gene. In some embodiments, the HDR template oligonucleotide additionally encodes a diagnostic restriction enzyme site. In some embodiments, the plasmid additionally encodes a reporter gene. In some embodiments, the Cas9 nuclease is Cas9n.

In some aspects, provided herein is a mammalian hematopoietic progenitor cell created by the methods described herein that does not express any Rh red blood cell antigen.

In some aspects, provided herein is a method for creating a mammalian hematopoietic progenitor cell that does not express any MNS red blood cell antigen, the method comprising the steps of: a) providing one or more guide RNAs designed to target a genes GYPA and GYPB; b) ligating the guide RNA of step (a) into a plasmid encoding a Cas9 nuclease; c) transfecting mammalian induced pluripotent stem cells with the plasmid of step (b); d) cloning and selecting the resulting clones that do not express the MNS antigens; and e) differentiating the selected clones into mammalian hematopoietic progenitor cells that do not express the MNS antigens. In some embodiments, the induced pluripotent stem cell comprises the GYPA and GYPB genes. In some embodiments, the mammalian induced pluripotent stem cell is transfected with the plasmid of step (b) in the presence of a homology-directed repair (HDR) template oligonucleotide. In some embodiments, the HDR template oligonucleotide encodes a stop codon to be introduced into the target genes. In some embodiments, the HDR template oligonucleotide encodes missense mutation in the target genes. In some embodiments, the HDR template oligonucleotide additionally encodes a diagnostic restriction enzyme site. In some embodiments, the plasmid additionally encodes a reporter gene. In some embodiments, the Cas9 nuclease is Cas9n.

In some aspects, provided herein is a mammalian hematopoietic progenitor cell created by the methods described herein that does not express any MNS red blood cell antigen.

In some aspects, provided herein is a method for creating a mammalian hematopoietic progenitor cell that does not express any Kell red blood cell antigen, the method comprising the steps of: a) providing one or more guide RNAs designed to target a genes selected from the group consisting of XK and KEL; b) ligating the guide RNA of step (a) into a plasmid encoding a Cas9 nuclease; c) transfecting mammalian induced pluripotent stem cells with the plasmid of step (b); d) cloning and selecting the resulting clones that do not express the Kell antigens; and e) differentiating the selected clones into mammalian hematopoietic progenitor cells that do not express the Kell antigens. In some embodiments, the induced pluripotent stem cell comprises the XK and KEL genes. In some embodiments, the mammalian induced pluripotent stem cell is transfected with the plasmid of step (b) in the presence of a homology-directed repair (HDR) template oligonucleotide. In some embodiments, the HDR template oligonucleotide encodes a stop codon to be introduced into the target genes. In some embodiments, the HDR template oligonucleotide encodes missense mutation in the target genes. In some embodiments, the HDR template oligonucleotide additionally encodes a diagnostic restriction enzyme site. In some embodiments, the plasmid additionally encodes a reporter gene. In some embodiments, the Cas9 nuclease is Cas9n.

In some aspects, provided herein is a mammalian hematopoietic progenitor cell created by the methods described herein that does not express any Kell red blood cell antigen.

In some aspects, provided herein is a method for creating mammalian cells that does not express any Rh red blood cell antigen, the method comprising the steps of: a) providing one or more guide RNAs designed to target a gene selected from the group consisting of RHD, RHCE, and RHAG; b) ligating the guide RNA of step (a) into a plasmid encoding a Cas9 nuclease; c) transfecting the mammalian cell with the plasmid of step (b), wherein the mammalian cell is selected from the group consisting of a mammalian pluripotent stem cell, a K562 erythro-leukemia cell, or a DAMI cell; d) cloning and selecting the resulting clones that do not express the Rh antigens; and e) expanding the selected clones in culture to produce mammalian cells that do not express the Rh antigens.

In some aspects provided herein is a method for creating mammalian cells expressing a specific platelet alloantigen, the method comprising the steps of: a) providing one or more guide RNAs designed to target platelet alloantigen target locus; b) ligating the guide RNA of step (a) into a plasmid encoding a Cas9 nuclease; c) transfecting the mammalian cell with the plasmid of step (b) in the presence of a homology directed repair template oligonucleotide, wherein the mammalian cell is selected from the group consisting of a mammalian pluripotent stem cell, a K562 erythro-leukemia cell, or a DAMI cell; d) cloning and selecting the resulting clones that do not express the platelet alloantigen of interest; and e) expanding the selected clones in culture to produce mammalian cells that express the platelet alloantigen of interest.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A: Depicts the strategy used to convert the P1^{A1} allelic form of GPIIIa to P1^{A2}, specifically, a pair of 20 bp gRNAs were designed to target the single-stranded nuclease, Cas9n, to opposite strands of the ITGB3 gene with a 13 bp offset surrounding the PI^{A} polymorphic site. The gRNAs were cloned into the BbsI site of the CRISPR vectors px461 or px462, which encode green fluorescent protein (GFP) or a puromycin-resistance gene, respectively, as well as Cas9n. The use of two different guides to direct the Cas9n nickase to nearby sites at this locus significantly reduces the incidence of off-target mutations relative to that incurred using a single guide RNA and the double-strand nuclease Cas9 (49, 50).

FIG. 1B: Depicts the strategy used to convert the PI^{A1} allelic form of GPIIIa to P1^{A2}, specifically, schematic illustration of the ITGB3 locus, showing the location of the two gRNA binding sites (orange bars) and the protospacer adjacent motifs (PAM) sequences (magenta), positioned 53 bp and 0 nucleotide upstream of the T>C mutation site, necessary to guide Cas9n to its cleavage site (red arrow heads). A 181 bp P1^{A2} HDR template was designed to introduce the Leu→Pro amino acid polymorphism. The T>C mutation responsible for the PI^{A1}/P1 polymorphism (highlighted in red) is flanked by 90 nucleotide homology arms, and creates an NciI site at the target locus that can be used for genotyping (13). The HDR template also contains two silent mutations (highlighted in blue) to prevent re-cleavage by Cas9n (see Methods).

FIG. 2A: Illustrates the conversion of P1^{A1}-homozygous DAMI cells to P1^{A2} using CRISPR/Cas9n-directed gene editing, specifically, DAMI cells were transfected with px461-gRNA1, px461-gRNA2, and a single-stranded P1-encoding HRD repair template using Nucleofection. GFP positive cells, representing ˜40% of the total population, were FACS-sorted 24 hrs post transfection, placed into cell culture, and expanded.

FIG. 2B: Illustrates the conversion of P1^{A1}-homozygous DAMI cells to P1 using CRISPR/Cas9n-directed gene editing, specifically, genomic DNA from cultured GFP-positive DAMI cells was isolated, PCR-amplified, and analyzed using the Surveyor nuclease. The red bracket indicates the range of expected fragment sizes. Note that the cell population that had been transfected with the two gRNAs shows the presence of insertions/deletions (indels), indicative of Cas9n-mediated cleavage at the P1^{A} locus.

FIG. 2C: Illustrates the conversion of P1^{A1}-homozygous DAMI cells to P1 using CRISPR/Cas9n-directed gene editing, specifically, genomic DNA from single cell GFP-positive DAMI clones was PCR amplified and digested with NciI to identify those clones encoding the P1^{A2} allelic isoform of GPIIIa. The red arrows indicate the expected NciI digestion products. Red asterisks indicate P1-positive clones #22 and #24.

FIG. 2D: Illustrates the conversion of P1^{A1}-homozygous DAMI cells to P1^{A2} using CRISPR/Cas9n-directed gene editing, specifically, the ITGB3 locus surrounding the P1^{A1}/P1^{A2} polymorphic site was PCR-amplified from genomic DNA of DAMI cell clones #22 and #24 and subjected to DNA sequence analysis, confirming the presence of the HDR-introduced T>C 29523 point mutation. The red arrow highlights the heterozygous partial allelic substitution expected in the multiploid DAMI cell line.

FIG. 2E: Illustrates the conversion of P1^{A1}-homozygous DAMI cells to P1^{A2} using CRISPR/Cas9n-directed gene editing, specifically, detergent cell lysates from wild-type and clone #24 DAMI cells were immunoprecipitated using the GPIIIa-specific mAb, AP3, followed by immunoblotting with human maternal anti-P1^{A2} antiserum. The relative equivalence of antigen loading was determined by immunoblotting whole cell lysates (WCL) with AP3 and anti-b-actin antibodies. Note that clone #24, but not wild-type DAMI cells, has a P1^{A2}-reactive band (red asterisk).

FIG. 3A: Illustrates the conversion of iPS cells from PI^{A1} to P1^{A2}, specifically, schematic of the diagnostic PCR reaction used to genotype the iPSCs. The NciI restriction enzyme site differentiates the PI^{A1} allelic isoform from P1^{A2}. Genomic DNA, isolated from iPS cells that had been transfected with px462-gRNA1, px462-gRNA2 and P1 ssODN and selected with puromycin, was PCR amplified and digested with NciI. Red arrows indicate the expected fragment sizes of a typical clone that had been converted to P1^{A2}.

FIG. 3B: Illustrates the conversion of iPS cells from P1^{A1} to P1^{A2}, specifically, sequencing data confirmed the T>C 29523 point mutation in CRISPR-edited P1^{A2} iPSCs. The red arrow indicates the target T>C mutation. The blue arrows indicate silent mutations that were intentionally introduced into the repair oligo to prevent digestion of the final edited genome by Cas9n.

FIG. 3C: Illustrates the conversion of iPS cells from P1^{A1} to P1^{A2}, specifically, allele-specific expression of GPIIb-IIIa (CD41) on both native and CRISPR-edited iPSC-derived day 8 hematopoietic progenitor cells. Non-adherent HPCs express abundant levels of the CD41/CD61 complex (integrin αIIb-β3) as well as CD235 (glycophorin A). Note that both cell lines were similarly double-positive.

FIG. 3D: Illustrates the conversion of iPS cells from P1^{A1} to P1^{A2}, specifically, cell lysates from wild-type, P1^{A1}-positive and CRISPR-edited P1^{A2} iPSC-derived HPCs were immunoprecipitated with AP3, followed by immunoblotting with human maternal anti-P1^{A2} antiserum. Note that the anti-P1^{A2} antiserum is positive for GPIIIa expressed in the gene-edited, but not native, iPS cell line (red arrow), while the P1^{A1}-specific mAb, SZ21, binds GPIIIa from native, but not gene-edited, iPS cells. Loading was evaluated by blotting with AP3 and anti-β-actin, as described in FIG. 2.

FIG. 4 shows DAMI cells transfected with px461-gRNA1, px461-gRNA2 and P1^{A2} ssODN using NUCLEOFECTION. Cells were analyzed 24 hrs post transfection using fluorescence and light microscopy.

FIG. 5 illustrates off-target analysis of gRNA1 and gRNA2. Top five putative off-target sites for gRNA1 and gRNA2 were PCR amplified from CRISPR-edited P1^{A2} iPS.K3 genomic DNA and directly sequenced. The possible off-target sequences were shown at the sixth to twenty eighth bases, as indicated by the capital letters above the sequencing peaks.

FIG. 6 shows the sequences and positions of on-target and possible off-target sites for gRNA1 and gRNA2. OT1: Off-target site for gRNA1. OT2: Off-target site for gRNA2. The primers for PCR amplification of off-target regions and expected size of PCR products are also listed.

FIGS. 7A-7B show generation of an HLA class I-negative iPSC founder line. (A) Schematic illustration of the B2M locus (SEQ ID NOs:95 and 96), showing the location of the gRNA binding site (orange bar) and the protospacer adjacent motifs sequence (magenta) necessary to guide Cas9 to its cleavage site (red arrow head). ATG start codon for b₂M translation is highlighted in red. Green arrow indicates an insertion or deletion (indel) is expected to be introduced into the genome through non-homologous end joining DNA repair pathway to cause a frameshift mutation in the B2M gene. (B) Flow cytometry analysis demonstrating the loss of surface expression of both b₂M and HLA in B2M knockout (KO) cells. APC, allophycocyanin; FITC, fluorescein isothiocyanate; WT, wild-type.

FIGS. 8A-8D show detection of anti-HPA-3a and HPA-3b alloantibodies using genetically edited iPSC-derived MKs. (A) Schematic illustration of donor plasmid and targeting strategy for converting HPA-3a to HPA-3b in B2MKO iPSCs. Red triangles flanking exon 26 of the ITGA2B gene indicate the 2 gRNA binding sites that will guide Cas9 to remove the entire exon encoding HPA-3a. HDR donor plasmid contains the removed sequence by Cas9 cleavage (orange box) with targeted T.G mutation responsible for HPA-3b conversion, flanked by 600-bp homology arms on each side (orange line). The recognition sequence and the PAM sequence of guide 2 (green line) are added to both ends of the homology arms for linearizing the donor templates in the transfected cells. Donor plasmid also contains silent mutations (blue X) to prevent re-cleavage by Cas9 and to generate an MfeI site for genotyping. (B) Genomic DNA, isolated from puromycin-resistant iPSC clones was amplified by PCR and digested with MfeI, which differentiates the HPA-3b allelic isoform from WT HPA-3a. Red arrows indicate the expected fragment sizes of a typical clone that had been converted to HPA-3b. (C) Sequencing data confirmed the T.G 13809 point mutation in CRISPR-edited HPA-3b iPSCs. (SEQ ID NO:97) The red arrow indicates the target T.G mutation. (D) Reactions of anti-HPA-3a and anti-HPA-3b patient sera with allele-specific iPSC-derived MKs in flow cytometric analysis. Both HPA-3a (gray) and HPA-3b (blue) iPSC lines were differentiated into CD41+/CD42b+ MKs. The MKs were incubated with patient sera followed by phycoerythrin (PE)-conjugated donkey anti-human immunoglobulin G (IgG). Anti-HPA-3a P3 patient serum did not contain anti-HLA class I antibody and was detectable only by using a whole-platelet assay in a clinical diagnostic laboratory. Other anti-HPA-3a and anti-HPA-3b patient sera were all clinically confirmed with MACE or MAIPA assays. For, forward; Rev, reverse.

FIGS. 9A-9D show detection of anti-HPA-9b alloantibodies using genetically edited iPSC-derived MKs. (A) Schematic illustration of a portion of the HDR template (SEQ ID NOs:57 and 98) and targeting strategy for converting HPA-9a to HPA-9b in HPA-3b iPSC clone. (SEQ ID NOs:99 and 100) The gRNA binding site (orange bar) and the PAM sequence (magenta) will guide Cas9 to its cleavage site (red arrow head) next to the HPA-9 allele. A 199-bp HPA-9b HDR template was designed to introduce the Val→Met amino acid polymorphism. The G.A mutation responsible for the HPA-9a/HPA-9b polymorphism (highlighted in red) is flanked by 99 nucleotide homology arms. Silent mutations (highlighted in blue) were introduced to prevent re-cleavage by Cas9 and create a PstI site at the target locus that can be used for genotyping. (B) Genomic DNA, isolated from puromycin-resistant iPSC clones was amplified by PCR and digested with PstI, which differentiates the HPA-9b allelic isoform from HPA-9a. Red arrow indicates the expected fragment sizes of a typical clone that had been converted to HPA-9b. (C) Sequencing data confirmed the G.A 13790 point mutation in CRISPR-edited HPA-9b iPSCs. (SEQ ID NO:101) The red arrow indicates the target G.A mutation. (D) Reactions of anti-HPA-9b patient sera with allele-specific iPSC-derived MKs in flow cytometric analysis. All of the HPA-3a (gray), HPA-3b (blue) and HPA-9b (red) iPSC lines were differentiated into CD41+/CD42b+ MKs. The MKs were incubated with patient sera followed by PE-conjugated donkey anti-human IgG. Anti-HPA-9b P1-P3 sera were clinically confirmed with either an MACE or an MAIPA assay. Anti-HPA-9b P4-P6 were HPA-9b suspected patient samples from clinically unresolved FNAIT cases.

FIG. 10 shows the HPA-3b gBlock fragment (SEQ ID NO:57).

FIG. 11 shows MK differentiation. Allele-specific iPSC-derived MKs were analyzed by flow cytometry to confirm the surface expression of CD41 and CD42b.

FIGS. 12A-12C show freeze-thawed iPSC-derived MKs preserve HPAs on the cell surface. (A-B) Anti-HPA-3a patient sera P4 (from Milwaukee) was tested with fresh or cryopreserved allele-specific iPSC-derived MKs in flow cytometric assay (A) and MACE assay (B). (C) Anti-HPA-9b P5 and P6 patient sera were tested with cryopreserved allele-specific iPSC-derived MKs in flow cytometric assay.

FIG. 13 shows GPIIb from both human platelets and iPSC-derived MKs contain sialylated T antigen. Cell lysates from human platelets and iPSC-derived MKs were immunoprecipitated with AP3 antibody, followed by neuraminidase treatment. The left panel shows T antigens with PNA blot. The middle and right panel show immunoblotting with anti-GPIIb or anti-GPIIIa antibody, respectively. Note that T antigens are only detectable on GPIIb after neuraminidase treatment to remove terminal sialic acids.

FIG. 14 shows examples of RBC antigens of interest in the methods described herein.

FIG. 15 shows an overview of the embodiments presented in Example 3.

FIG. 16 shows embodiments of RBC antigen surface targets of the methods described herein.

FIG. 17 shows a schematic of protein assembly on the RBC surface. Several proteins complexes on the red cell surface carrying antigens for glycophorins A, B, C, and D (labeled GPA, GPB, GPC and GPD). Proteins carrying Duffy and Kell red cell antigens are also shown.

FIG. 18 shows evaluation of guide RNA pairs using K562 model cells before using iPS cells.

FIG. 19 shows a schematic of the Rh system complex.

FIG. 20 shows successful deletion (knockout) of the RhAG gene in iPS cells.

FIG. 21 shows the protocol for differentiating hematopoietic progenitor cells to the erythroid lineage.

FIG. 22 shows differentiation into RBCs and demonstration that RhAG has been lost from the cell surface.

FIG. 23 shows RhAG knockout cells also fail to express RhD and RHCE.

FIG. 24 shows an example of an antibody identification panel.

FIG. 25 shows a schematic on an antibody identification panel method.

FIG. 26 shows an example of antibody panel interpretation.

FIG. 27 shows examples of red blood cell blood group systems.

FIG. 28 shows how RBC blood group systems correspond to antibody identification panels.

FIG. 29 shows exemplary embodiments of an RBC antibody identification panel of the present disclosure.

FIG. 30 shows the day 0 test for iPS cell pluripotency markers.

FIG. 31 shows differentiation of HPCs from iPS cells.

FIG. 32 shows identification of CD41+CD235+ HPCs.

FIG. 33 shows expression of antigens and blood group systems on differentiated erythroblasts.

FIG. 34 shows an exemplary vector for gRNA expression.

FIG. 35 shows confirmation of gene editing in K562 cells using flow cytometry.

FIG. 36 shows the antigens of the MNS blood group system. (SEQ ID NOs:102 and 103)

FIG. 37 shows the knockout of MNS blood group antigens by targeting the GYPA and GYPB genes.

FIG. 38 shows the antigens of the Kell blood group system.

FIG. 39 shows the knockout of the Kell blood group system antigens by targeting the XK gene.

### DESCRIPTION OF THE INVENTION

In General

Human platelet alloantigens (HPAs) reside on functionally important platelet membrane glycoproteins and are caused by single nucleotide polymorphisms in the genes that encode them. Antibodies that form against HPAs are responsible for several clinically important alloimmune bleeding disorders, including fetal and neonatal alloimmune thrombocytopenia, posttransfusion purpura, and multitransfusion platelet refractoriness.

The HPA-1a/HPA-1b alloantigen system, also known as the P1^{A1}/PI^{A2} polymorphism, is the most frequently implicated HPA among Caucasians, and a single C29523T nucleotide substitution, resulting in a Leu33Pro amino acid polymorphism within the PSI domain of the integrin β3 subunit (platelet glycoprotein IIIa) was shown 25 years ago to be responsible for generating the HPA-1a/HPA-1b alloantigenic epitopes. Like other low-frequency alloantigens, HPA-1b/b platelets are relatively rare in the population, and therefore often difficult to obtain for purposes of transfusion therapy and diagnostic testing.

The platelet alloantigen system has had a variety of nomenclature styles over the years since it was first documented by one of the inventors on this application. The human platelet alloantigen or HPA nomenclature is the most widely used today. However, historically, the antigens were known by names such as Pla, Pen, Bak, Br, Gov, and others. Certain of the alloantigen mutations occur more frequently in nature, leading to a higher incidence of clinical issues associated with that polymorphism.

Below is a listing of HPA alloantigens suitable for the present invention, the glycoprotein impacted, and their genetic basis:

| | Glyco- | Hugo Gene | | Nucleotide | | Mature |
|---|---|---|---|---|---|---|
| Antigen | protein | Nomenclature | Chromosome | Change | Precursor | Protein |
| HPA-1 | GPIIIa | ITGB3 | 17 | 176T > C | L59P | L33P |
| HPA-2 | GPIba | GP1BA | 17 | 482C > T | T161M | T145M |
| HPA-3 | GPIIb | ITGA2B | 17 | 2621T > G | I874S | I843S |
| HPA-4 | GPIIIa | ITGB3 | 17 | 506G > A | R169Q | R143Q |
| HPA-5 | GPIa | ITGA2 | 5 | 1600G > A | E534K | E505K |
| HPA-6w | GPIIIa | ITGB3 | 17 | 1544G > A | R515Q | R489Q |
| HPA-7w | GPIIIa | ITGB3 | 17 | 1297C > G | P433A | P407A |
| HPA-8w | GPIIIa | ITGB3 | 17 | 1984C > T | R662C | R636C |
| HPA-9w | GPIIb | ITGA2B | 17 | 2602G > A | V868M | V837M |
| HPA-10w | GPIIIa | ITGB3 | 17 | 263G > A | R88Q | R62Q |
| HPA-11w | GPIIIa | ITGB3 | 17 | 1976G > A | R659H | R633H |
| HPA-12W | GPIbb | GP1BB | 22 | 119G > A | G40E | G15E |
| HPA-13W | GPIa | ITGA2 | 5 | 2483C > T | T828M | T799M |
| HPA-14W | GPIIIa | ITGB3 | 17 | 1909_1911de | K637del | K611del |
| | | | | IAAG | | |
| HPA-15 | CD109 | CD109 | 6 | 2108C > A | S703Y | S682Y |
| HPA-16W | GPIIIa | ITGB3 | 17 | 497C > T | T166I | T140I |
| HPA-17W | GPIIIa | ITGB3 | 17 | 662C > T | T221M | T195M |
| HPA-18W | GP1a | ITGA2 | 5 | 2235G > T | Q745H | Q716H |
| HPA-19W | GPIIIa | ITGB3 | 17 | 487A > C | K163Q | K137Q |
| HPA-20W | GPIIb | ITGA2B | 17 | 1949C > T | T650M | T619M |
| HPA-21W | GPIIIa | ITGB3 | 17 | 1960G > A | E654K | E628K |
| HPA-22bw | GPIIb | ITGA2B | 17 | 584A > C | K195T | K164T |
| HPA-23bw | GPIIIa | ITGB3 | 17 | 1942C > T | R648W | R622W |
| HPA-24bw | GPIIb | ITGA2B | 17 | 1508G > A | S503N | S472N |
| HPA-25bw | GPIa | ITGA2 | 5 | 3347C > T | T1116M | T1087M |
| HPA-26bw | GPIIIa | ITGB3 | 17 | 1818G > T | K606N | K580N |
| HPA-27bw | GPIIb | ITGA2B | 17 | 2614C > A | L872M | L841M |
| HPA-28bw | GPIIb | ITGA2B | 17 | 2311G > T | V771L | V740L |
| HPA-29bw | GPIIIa | ITGB3 | 17 | 98C > T | T33M | T7M |

Our examples below disclose one embodiment of the present invention. As a first step in producing designer platelets expressing low-frequency human platelet alloantigens, we employed a CRISPR/Cas9 RNA-guided nicking nuclease system to transform megakaryocyte-like cells expressing the Leu33 allele of integrin β3 to the Pro33 form. Two different guide RNAs that target the ITGB3 gene with a 13-base pair offset 53 bases and 0 nucleotides upstream of the C/T polymorphism site were designed and cloned into plasmids that co-express GFP as well as a mutated form of Cas9 that nicks only one strand of DNA (Cas9n). Such a double-nicking strategy has been shown in other systems to increase the specificity of gene targeting while minimizing off-target effects.

A 200 bp single-stranded DNA oligonucleotide encompassing the single base C29523T mismatch was also synthesized to be used for homology-directed repair (HDR) of the endogenous ITGB3 gene sequence. The HDR oligo was then transfected, together with the two plasmids encoding the guide RNAs+Cas9n+GFP, into megakaryocyte-like DAMI cells. Twenty-four hours post-transfection, GFP positive cells were sorted by flow cytometry and isolated as single clones.

Surveyor endonuclease assays revealed that ˜30% of the GFP positive clones had been cleaved at the expected location, indicating efficient double nicking directed by the pair of guide RNAs. Additionally, two out of twenty seven isolated clones had incorporated the HDR repair template, as reported by a diagnostic NciI restriction enzyme site that is specific for the T29523-bearing HPA-1b allele. Sequence analysis further confirmed conversion of C29523 to T in these two clones. Finally, Western blotting using HPA-1b-specific human alloantisera verified that these DAMI cells now expressed the HPA-1b (P1^{A2}) alloantigenic epitope. Taken together, these results establish that the CRISPR/Cas system can be successfully employed to genetically edit this and other clinically-important HPAs in human cells. Application of this technology for the generation of alloantigen-specific human induced pluripotent stem cells holds great potential as a general tool for producing designer platelets for diagnostic and therapeutic use.

### Embodiments of the Present Invention

In one embodiment, the present invention is the creation of designer platelets or red blood cells via use of gene editing and pluripotent cell culture techniques. In a broad example of the present invention, one would use any gene editing technique to insert an alloantigen of interest in a pluripotent cell and culture and/or differentiate the cell to create the cell type desired. In a preferred embodiment, the cells would be manipulated to be devoid of HLA class I molecules.

In one embodiment, the present invention is a method to create alloantigen-specific platelets using CRISPR/Cas 9 gene editing strategies. The method relies upon existing CRISPR/Cas9 methods in combination with existing pluripotent cell culture methods. In the most preferred embodiment, the cells would be additionally edited to remove the β₂ microglobulin gene responsible for expression of HLA on the surface of platelets. The resulting platelets would be useful in laboratory testing or transfusion.

Cells devoid of HLA would be especially useful in diagnostic testing which seeks to determine the presence of patient antibody to platelet alloantigens. In current methods, multiple sources of platelets carrying varying HLA types need to be used to rule out potential cross reactions of patient antibody with the specific platelet alloantigen versus antibodies to HLA on the surface of the platelet. These novel cells devoid of HLA would offer a benefit to simplify laboratory testing.

The inventors have discovered a method of creating platelets expressing specific platelet alloantigens (“designer platelets”) by combining gene editing techniques, preferably CRISPR/CAS-9 gene editing techniques, and cell culture techniques employing pluripotent cells, such as iPS or DAMI cells. In one embodiment, the method comprises the steps of: a) combining two guide RNAs targeting ITGB3 gene around a platelet alloantigen locus of interest, such as the PI^{A1} locus; b) adding a repair template which carries the targeted mutation flanked by a homology arm on each side which creates a restriction enzyme site, preferably an NciI site, at the target locus; c) ligating the guide sequences into a plasmid which also expresses a nuclease capable of cleaving double stranded nucleotides, such as Cas9n, and a reporter gene, such as GFP; d) transfecting pluripotent cells, such as iPS cells, or DAMI cells with the nuclease/guide RNA plasmids in the presence of the HDR repair oligo; e) cloning, expanding and testing the resulting reporter-positive clones for expression of the alloantigen transgene of interest; and f) culturing the resulting cells in an appropriate developmental manner so that platelets are expressed in the culture.

In a preferred embodiment, the cells are genetically manipulated to express the platelet antigen of interest and would be additionally manipulated to be devoid of HLA class I molecules. This is advantageous to rule out issues of cross-reactivity with antibodies from a patient sample or other issues of HLA compatibility in a patient. One practical way to accomplish this embodiment would be to first produce the pluripotent or DAMI cell of interest that has been manipulated to have the β2 microglobulin gene of HLA (responsible for HLA expression) removed. The cells could then be then further edited to express the platelet isotypes of interest. β2 microglobulin guide RNAs are available commercially from sources such as Santa Cruz Biotechnologies and, thus, β2 microglobulin deficient cells can be produced by following manufacturer instructions.

In one preferred embodiment, one would use pluripotent or iPS cell growth and differentiation conditions that favored the myeloid or megakaryocytic lineages. By using a cell type that is a precursor to red cells or platelets, one could better reproduce the carbohydrate glycosylation patterns of the surface glycoproteins of antigenic interest, thereby creating an epitope that more closely resembles naturally occurring alloantigens.

In another embodiment, one could delete the β2 microglobulin gene of HLA and add the platelet isotype of interest at the same time during the gene editing process. Briefly, the method comprises the steps of: a) combining two guide RNAs targeting ITGB3 gene around a platelet alloantigen locus, such as the PI^{A1} locus, along with one or more guide RNAs targeting the areas flanking the β2 microglobulin gene of HLA; b) adding the repair template which carries the targeted mutation flanked by a homology arm on each side which creates an at the target locus; c) ligating the guide sequences into a plasmid which also expresses a nuclease, such as Cas9n, and a reporter gene, such as GFP; d) transfecting iPS or DAMI cells with the nuclease/guide RNA plasmids in the presence of the HDR repair oligo; e) cloning, expanding and testing the resulting reporter positive clones for expression of the alloantigen transgene of interest and the deletion of the β2 microglobulin gene; and f) culturing the resulting cells so that the designer platelets are expressed in the culture.

In another embodiment, the invention is a method of creating “Designer Red Cells” utilizing the same gene editing approach. Red cells comprise a variety of antigens on their surface which include ABO, RhD, and the following:

RhCE: C(RH2), E(RH3),c(RH4), e(RH5),CW(RH8), V(RH10), hrS(RH19), VS(RH20), hrB(RH31)

Kell: K(KEL1), k(KEL2), Kpa(KEL3), Kpb(KEL4), Jsa(KEL6), Jsb(KEL7)

Kidd: Jka(Jk1), Jkb(Jk2), JKB_null(IVS5-1a), JKB_null(871C)

Duffy: Fya(FY1), Fyb(FY2), FYB_GATA, FYB[265T]_FYX

MNS: M(MNS1), N(MNS2), S(MNS3), s(MNS4), U(MNS5), Mia(MNS7)

Diego: Dia(DI1), Dib(DI2)

Dombrock: Doa(DO1), Dob(D02), Hy(DO4), Joa(DO5)

Colton: Coa(CO1), Cob(CO2)

Cartwright: Yta(YT1), Ytb(YT2)

Lutheran: Lua(LU1), Lub(LU2)

Briefly, the method comprises the steps of: a) combining two guide RNAs targeting one or more of the above-listed red cell genes; b) adding the repair template which carries the targeted mutation flanked by a homology arm on each side which creates a restriction site, such as an NciI site, at the target locus; c) ligating the guide sequences into a plasmid which also expresses a nuclease and a reporter gene such as GFP; d) transfecting pluripotent cells or DAMI cells with the nuclease/guide RNA plasmids in the presence of the HDR repair oligo; e) cloning, expanding and testing the resulting reporter positive clones for expression of the alloantigen transgene(s) of interest; and f) culturing the resulting cells in a developmentally suitable manner so that designer red cells are expressed in the culture.

In another embodiment, the invention is a method of using the resulting pluripotent cells cells, cell derivatives, designer red cells, or designer platelets in the laboratory as reagents to test patient blood samples for the presence of antibody to the expressed alloantigens or as controls for nucleic acid testing of those genes.

In another embodiment, the invention is a method of creating designer platelets for use in transfusion of patients with platelets of a specific isotype. For example, one could transfuse gene-edited, alloantigen specific designer platelets or their progenitor cells into patients for the purpose of correcting thrombocytopenia and similar bleeding disorders.

In another embodiment, the invention is a method of creating designer platelets or red cells for use in diagnostic testing through solubilizing a gene-edited pluripotent cell or progeny cells with a detergent and linking those solubilized alloantigen proteins to a solid surface such as a bead or plate. This solid surface, most preferably a bead, carrying the platelet alloantigen would serve as a platelet for the purpose of detection platforms such as flow cytometry and others. A solid surface, such as an ELISA plate, would serve as a platelet for the purpose of detection platforms such as ELISA and others. A variety of detergents could be used that include both non-ionic or ionic detergents that onecould find by empirical testing. Common detergents used for this purpose include CHAPS, Tween®20, Triton X 100 and others.

A preferred method uses Cas9n as a nuclease because it relies on single nucleic acid chain break resulting in a higher efficiency of clones produced. However, other Cas family nucleases, or family of nucleases could be used. Other suitable nucleases, such as Cpf1, zinc finger nucleases, and talens, could be used though additional nucleases with similar properties are in development.

In another embodiment, the invention is a method of using the resulting pluripotent cells, cell derivatives, designer red cells or designer platelets in the laboratory as reagents to test patient blood samples for the presence of antibody. One would do this by using the designer platelets or designer red cells as the source of antigen to then test for binding of patient-derived antibodies from a blood sample. The designer red cell or designer platelet would serve as a solid surface for the patient antibody to bind.

Any blood sample could serve as a source of patient antibody for the purpose of detecting patient antibody titers to a specific platelet alloantigen. In terms of detection and quantification of the patient antibody, methods such as dilution titration, dose response curves, and the use of a secondary antibody directed to the patient antibody known by those of skill in the art could be used. Detection platforms could include enzyme linked immunosorbend assays (ELISAs), western blotting, direct or indirect microscopy, flow cytometry or other methods known in the art.

In another embodiment, the designer red cell or designer platelet would serve as a source of control reagents for either nucleic acid or antigenic analysis. At times, it is difficult to source patients with very rare alloantigens. These patient samples are needed as controls for both nucleic acid or DNA testing of patient materials and for antigenic testing of patient materials.

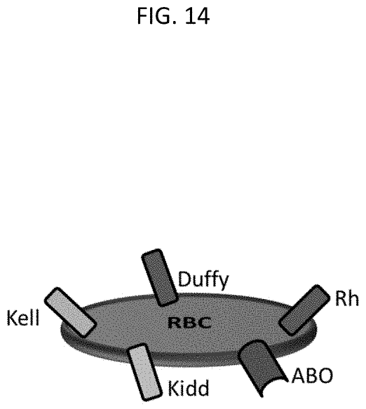

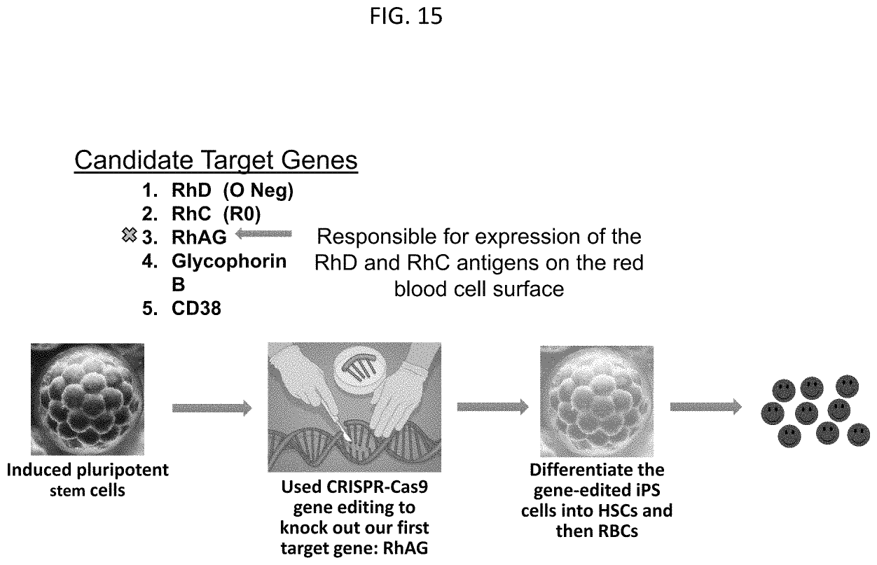

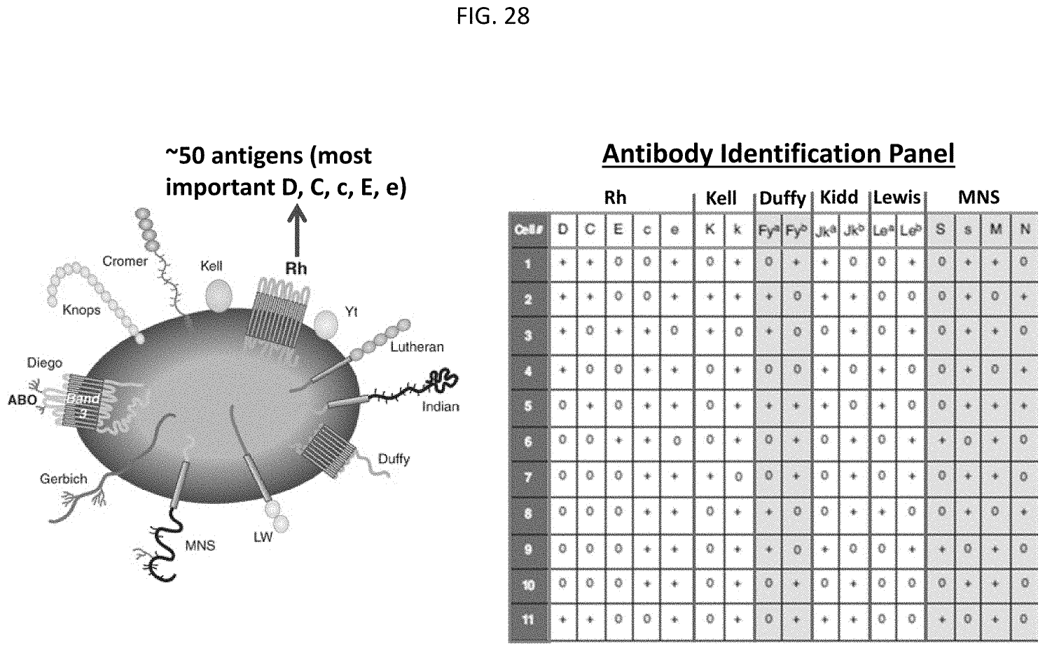

The International Society of Blood Transfusion (ISBET) recognizes 36 blood group systems including more than 350 different antigens. As used herein, “blood system group” refers to the recognized groups of one or more antigens that are controlled at a single gene locus or by two or more very closely linked loci. Common red cell antigens belong to, but are not limited to, the Kell, Duffy, Rh, ABO, Kidd, MNS, P, Lutheran, Lewis, Diego, Dombrock, Colton, and Cartwright blood system groups. (FIGS. 14 and 28). The antigens assigned to each blood system group, as well as the genes encoding the antigens, are known and described in the art. See, for example, Mitra et al. (“Blood group systems,” Indian J. Anaesth., 2014, 58(5):524-528).

Blood Group Systems

| | Number | | |
|---|---|---|---|
| Name | of Antigens | Gene Name | Chromosome |
| ABO | 4 | ABO | 9 |
| MNS | 43 | GYPA, GYPB, GYPE | 4 |
| P | 1 | P1 | 22 |
| Rhesus (Rh) | 49 | RHD, RHCE | 1 |
| Lutheran | 20 | LU | 19 |
| Kell | 25 | KEL | 7 |
| Lewis | 6 | FUT3 | 19 |
| Duffy | 6 | FY | 1 |
| Kidd | 3 | SLC14A1 | 18 |
| Diego | 21 | SLC4A1 | 17 |
| Dombrock | 7 | ART4 | 12 |
| Colton | 2 | AQP1 | 7 |
| Cartwright (Yt) | 2 | ACHE | 7 |

In some embodiments, the target gene of interest is RHD, RHC, RhAG, CD38, XK, GYPA, GYPB, GYPE, P1, LU, KEL, FUT3, FY, SLC14A1, SLC4A1, ART4, AQP1, and ACHE.

In some embodiments, the methods described herein are used to modify or delete all or a portion of the Rh blood group system. After the ABO blood group system, the Rh blood group system is the most important. The Rh blood group system includes 49 defined blood group antigens, including D, C, c, E, and e. Expression of the RhD antigen, or lack thereof, is common describe as a “positive” or “negative” cell type in an individual. In other words, an individual with the RhD antigen as a positive blood type, whereas an individual without the RhD antigen has a negative blood type. The Rh family includes proteins RhD, RhCE, RhAG, RhBG, and RHCG encoded by their respective genes RHD, RHCE, RHAG, RHBG, and RHCG.

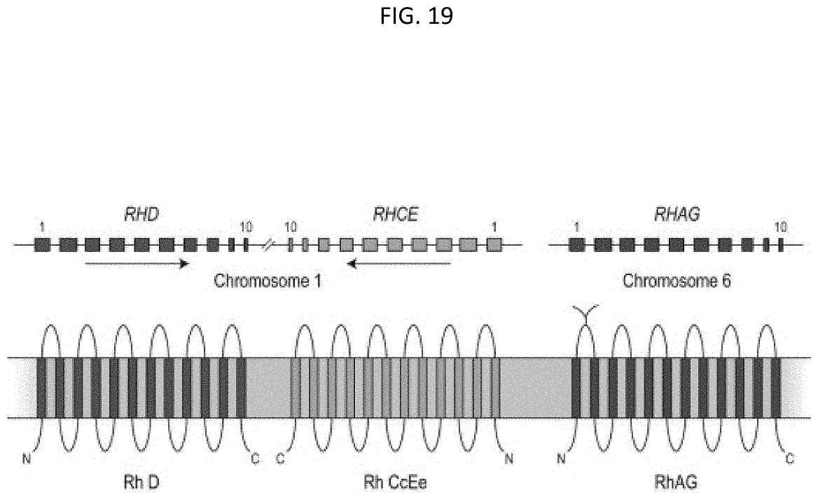

The RhD antigen is also known as Rh polypeptide 1 (RhP1) or cluster of differentiation 240D (CD240D) and is encoded by the RHD gene. RhD and RhCE differ by only 36 amino acids. Each of RhD and RhCE are 12-pass transmembrane proteins (FIG. 19).

The RHAG gene encodes RhAG, which is responsible for bringing the immunogenic Rh blood group system proteins RhD and RhCE to the RBC surface. However, RhAG itself is not polymorphic and does not include any of the Rh blood system antigens. Without wishing to be bound by any particular theory or embodiment, disruption, deletion, or mutation of RHAG gene will disrupt presentation of the RhD and RhCE antigens on the red blood cell surface. Effectively, disruption, deletion, or mutation of the RHAG gene will disrupt or eliminate the presentation of the RhD and RhCE antigens on the RBC surface and therefore produce an RBC without the RhD and RhCE antigens. In some embodiments, the methods described herein are used to knockout the Rh blood group system proteins RhD and RhCE by introducing a missense mutation within the RHAG gene. In some embodiments, the methods described herein are used to knockout the Rh blood group system proteins RhD and RhCE by deleting a portion of the RHAG gene.

In some embodiments, the methods described herein are used to knockout the Rh blood group system proteins RhD and RhCE by deleting a portion or the entirety of each of the RHD and RHCE genes.

In some embodiments, the methods described herein are used to modify or knockout surface expression of the protein carrying the Kell polymorphism. Specifically, a missense mutation or deletion is engineered within the KX gene on chromosome X encoding Kx, whereby the Kell antigen is not presented on the RBC surface. Mutations in the XK gene are associated with McLoed syndrome, which is an X-linked recessive disorder characterized by abnormalities in the neuromuscular and hematopoietic systems. Kell antigens are very weakly expressed on XK negative RBCs. Therefore, mutation within or deletion of the XK gene will create an iPS cell that may be differentiated into a hematopoietic progenitor cell that does not express the Kell blood group system antigens. To mutate or knockout the Kell blood group system antigens, an iPS cell is transfected with a vector encoding guide RNAs targeting the XK gene and encoding a Cas9 nuclease. Following transfection, cells in which a portion or the entire XK gene has been deleted or mutated are expanded and differentiated into hematopoietic progenitor cells and RBCs.

In some embodiments, the methods described herein are used to modify or knockout surface expression of MNS blood group system antigens. The antigens of the MNS blood group system are found on extracellular portions of the glycophorin A and glycophorin B proteins, which are encoded the glycophorin A (GYPA) and glycophorin B (GYPB) genes, respectfully. Therefore, mutation within or deletion of the GYPA and GYPB genes will create iPS cells that may be differentiated into a hematopoietic progenitor cell that does not express the MNS blood group system antigens. To mutate or knockout the MNS blood group system antigens, an iPS cell is transfected with a vector encoding guide RNAs targeting the GYPA and GYPB genes and encoding a Cas9 nuclease. Following transfection, cells in which a portion or all of the GYPA and GYPB genes have been mutated or deleted are expanded and differentiated into hematopoietic progenitor cells and RBCs.

In some embodiments, selection of clones that do not express the recited antigens may be done by sequencing the target gene to confirm deletion or mutation. In some embodiments, selection of clones that do not express the recited antigens may be done using flow cytometry.

In some embodiments, RBC antigen expression is disrupted by introducing a missense mutation or a stop codon into the coding sequence of a gene encoding the antigen. For example, a homology directed repair templet oligo nucleotide can be introduced while transfecting the iPS cell with the vector encoding the guide RNA and the Cas9 nuclease.

### Definitions

By “blood sample” we mean whole blood, plasma, sera, platelet rich plasma or other products which can be created by fractionating or purifying products from a blood product. This would include but not be limited to such products as cryopresserved blood products or antibody purified from any of the aforementioned blood products. Blood samples could be used as a source of patient antibody.

By “cell type of interest” we mean platelets, red cells, progenitor cells, stem cells, or alloantigens produced by gene editing methods described herein which are then bound to a solid surface such as a bead or microtiter plate to create a cell substitute.

By “designer platelets” we mean a platelet that is the result of genetic editing so that it expresses a specific platelet antigen or group of antigens on its surface, an iPS cell or iPS cell derivative that expresses a specific platelet antigen or group of antigens on its surface, or a platelet antigen from any of the aforementioned cell types that is subsequently bound to a solid surface such as a microtiter plate or bead to create a surface similar to a platelet with respect to platelet antigen expression. Designer platelets could be made to lack expression of HLA or other surface antigens by means of additional gene editing.

By “designer red blood cells” or “designer red cells” we mean a cell that is the result of genetic editing so that is expresses a specific red blood cell antigen or group of antigens on its surface and/or is devoid of one or more red blood cell antigen or group of antigens. A designer red blood cell may be an engineered iPS cell derived erythrocyte or erythrocyte precursor cell, a genetically modified human erytholeukemic cell line such as K562, an erythroblast cell line such as TF-1, another genetically edited leukemic cell line capable of red blood cell antigen surface display or red blood cell antigen expression, or an antigen from any of the aforementioned cell types that is subsequently bound to a solid surface such as a microtiter plate or bead to create a surface similar to a cell with respect to red cell antigen expression.

By “gene editing” we mean any number of enzyme systems that one could use to perform gene editing which include; CRISPR/Cas (Clustered regularly interspaced short palindrome repeats (CRISPRs)) and CRISPR-associated Zinc-finger nucleases (ZFNs); and transcription-activator-like effector nucleases (TALENs). These are chimeric nucleases composed of programmable, sequence-specific DNA-binding modules linked to a nonspecific DNA cleavage domain.

By “guide sequence” we mean short pieces of RNA complementary to the DNA sequence to be edited which provide both targeting and scaffolding or binding ability for an enzyme.

By “HDR repair oligo” we mean homology-directed repair oligonucleotide to accomplish a template-dependent DNA break repair. By supplying a homology-containing donor template along with a site specific nuclease, HDR faithfully inserts the donor molecule at the targeted locus. This approach enables the insertion of single or multiple transgenes, as well as single nucleotide substitutions as is the case for the alloantigen edits which are the subject of the present application.

By “homology arm” we mean that piece of the repair template that is responsible for pairing targeting the repair template to the portion of DNA to be edited. Homology arms can have varying lengths and are most typically 50-80 bases in length.

By “pluripotent cells” we mean to include cells with the developmental possibility of multiple lineages, including induced pluripotent stem cells, bone marrow, DAMI cells, progenitor cells, human embryonic stem cells, or any cell capable of differentiating and being grown in cell culture.

By “repair template” we mean a piece of DNA which provides the edited DNA to be incorporated into the genome.

### EXAMPLES

### Example 1

Human platelet alloantigens (HPAs) reside on functionally important platelet membrane glycoproteins, and are caused by single nucleotide polymorphisms in the genes that encode them. Antibodies that form against HPAs are responsible for several clinically important alloimmune bleeding disorders, including fetal and neonatal alloimmune thrombocytopenia and post-transfusion purpura. The HPA-1a/HPA-1b alloantigen system, also known as the P1^{A1}/PI^{A2} polymorphism, is the most frequently implicated HPA among Caucasians, and a single Leu33Pro amino acid polymorphism within the integrin β3 subunit is responsible for generating the HPA-1a/HPA-1b alloantigenic epitopes. HPA-1b/b platelets, like those bearing other low-frequency platelet-specific alloantigens, are relatively rare in the population, and difficult to obtain for purposes of transfusion therapy and diagnostic testing. We employed CRISPR/Cas9 gene editing technology to transform Leu33-positive megakaryocyte-like DAMI cells and induced pluripotent stem (iPS) cells to the Pro33 allotype. CD41-positive megakaryocyte progenitors derived from these cells expressed the HPA-1b (P1^{A2}) alloantigenic epitope, as reported by diagnostic NciI restriction enzyme digestion, DNA sequencing, and western blot analysis using HPA-1b-specific human maternal alloantisera. Application of CRISPR/Cas9 technology to genetically edit this and other clinically-important HPAs holds great potential for production of designer platelets for diagnostic, investigative and ultimately therapeutic use.

In addition to their well-described roles in platelet adhesion and thrombus formation, each of the major human platelet membrane glycoproteins exists in the human gene pool in multiple allelic isoforms, most of which differ from the predominant wild-type allele by only a single amino acid. A subset of these polymorphic isoforms is immunogenic in man—i.e. the three-dimensional structures encompassing the polymorphic amino acid are capable of eliciting an alloimmune response in appropriately mis-matched individuals. The resulting alloantibodies bind to exposed target epitopes on the platelet surface, resulting in rapid clearance from circulation of the opsonized platelets by liver and splenic macrophages (1).

Alloantibodies to platelet-specific antigens are responsible for two clinically-important bleeding disorders: Post-transfusion purpura (PTP) and neonatal alloimmune thrombocytopenia (NAIT—variously referred to in the literature as NATP, FNIT, and FNAIT) (2). PTP is a rare syndrome in which a multiparous woman, after receiving a blood transfusion, enigmatically clears not only the transfused platelets, but her own as well, leading to severe thrombocytopenia, bruising, and petechiae. Unlike PTP, NAIT is a fairly common disorder, complicating 1 in 350 pregnancies (3), and leading to severe fetal and/or neonatal thrombocytopenia in approximately 1 in 1000 births (3, 4). Although many infants recover uneventfully, NAIT is the leading cause of severe thrombocytopenia in the fetus and neonate, often producing bleeding serious enough to require transfusion with “antigen-negative” platelets. The most destructive consequences of NAIT, however, are intracranial hemorrhage and intrauterine death as early as 20-24 weeks of gestation (5). Despite advances in treatment, NAIT remains the leading cause of intracranial hemorrhage in full-term infants (6-10), often leading to life-long disability.

The first human platelet alloantigen system was identified serologically more than 50 years ago and termed Zw (11) or Platelet Antigen 1 (PI^{A1}) (12) respectively. The N^{A} epitope is controlled by a single Leu33Pro amino acid polymorphism within the PSI domain of platelet membrane glycoprotein (GP)IIIa (=the integrin β3 subunit) (13), and work performed in many laboratories since that time has led to the identification of 29 distinct Human Platelet-specific Alloantigen (HPA) systems (HPAs 1-29) on six different glycoproteins (14). PI^{A1} (HPA-1a), however, remains the alloantigen that most commonly provokes PTP and NAIT, being responsible for ˜80% of the cases in which an alloantibody can be detected.

Despite the availability of numerous DNA-based platforms for the rapid genotyping of each of the HPAs (15-19), identification of a platelet alloantigen-specific antibody in the maternal sera is still required to make a positive diagnosis of NAIT (10), and less commonly, for posttransfusion refractoriness (20). Determination of antibody specificity, and in some cases titer, is also critical for guiding prenatal treatment to reduce the likelihood of prenatal bleeding and intracranial hemorrhage in utero, facilitating postnatal management, and managing future pregnancies (10, 21, 22). Platelets bearing low-frequency platelet alloantigens, however, are often difficult or impossible to obtain, and their lack of availability represents a significant barrier for developing effective therapies, and for diagnosing, NAIT. The purpose of the present investigation was to combine recent advances in gene editing and platelet production technologies to generate antigenically-distinct, alloantigen-specific megakaryocyte progenitors for diagnostic and investigative use.

Results

CRISPR-mediated conversion of PI^{A1} homozygous DAMI cells to P1^{A2}. Because induced pluripotent stem (iPS) cells do not express the GPIIb-IIIa (CD41/CD61) complex unless they are subjected to a rather lengthy differentiation process, conditions for CRISPR-mediated genome editing, including selection of guide RNAs (gRNAs) and homology directed repair (HDR) oligonucleotides, were first optimized using DAMI cells; a human polyploid megakaryocytic cell line that constitutively expresses the common PI^{A1} allelic isoform of GPIIIa (23).

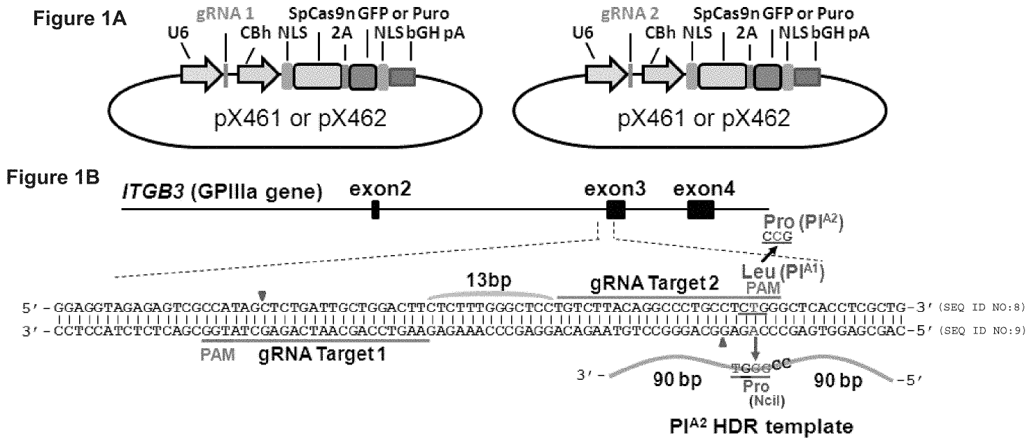

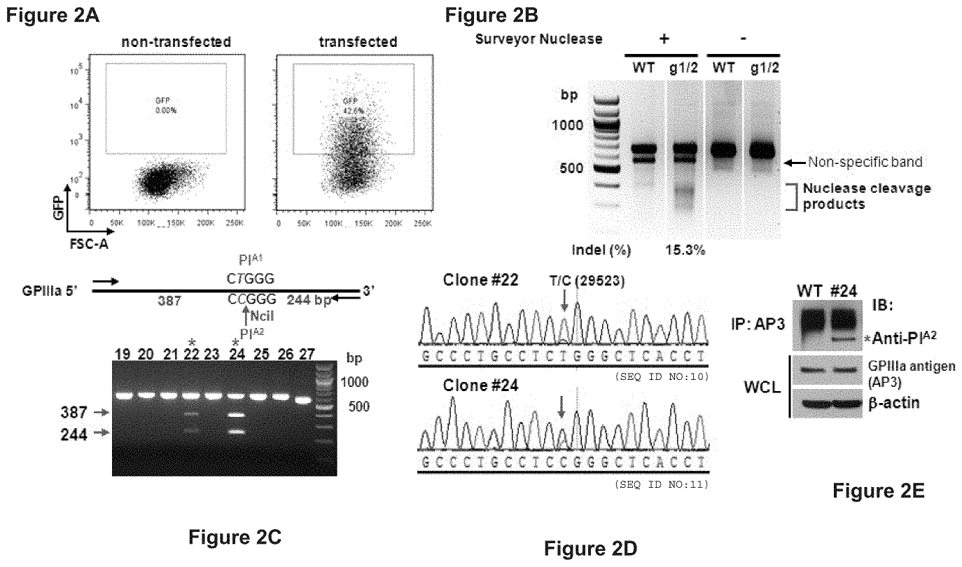

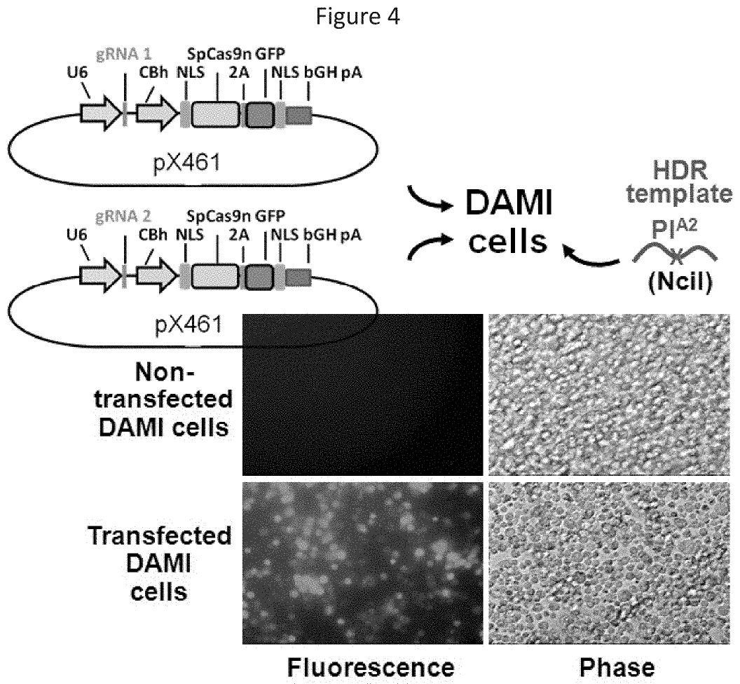

To convert the PI^{A1} allelic form of GPIIIa, which differs from P1 by a single T29523C nucleotide substitution in the ITGB3 gene, to P1^{A2}, we designed two gRNAs targeting opposite strands of ITGB3 gene (FIG. 1B) and introduced them into px461, which encodes the single-strand nickase Cas9n and green fluorescent protein (GFP) (FIG. 1A). GFP-encoding px461 plasmids harboring each gRNA sequence were transfected into DAMI cells together with a 181 nucleotide P1^{A2} HDR template (FIG. 4), and the resulting GFP positive cells were sorted by flow cytometry to enrich for transfected cells (FIG. 2A). Following cell expansion, Surveyor nuclease digestion of a genomic DNA hybridized/re-hybridized PCR amplicon spanning the Cas9n cleavage site revealed partial cleavage products of 270-371 bp (FIG. 2B), indicating efficient gRNA-directed double nicking by Cas9n. Genomic DNA from 27 GFP-positive single cell clones was digested with NciI, revealing two clones (#22 and #24) that carried the P1^{A2} polymorphism (FIG. 2C).

DNA sequence analysis (FIG. 2D) confirmed heterozygous replacement of the P1^{A2} HDR template in these cells. Based on the band intensity of the NciI cleavage products, it appears that approximately half of the PI^{A1} alleles in clone #24 were CRISPR-converted to P1^{A2}, while only one fourth were converted in clone #22—expected due to the polyploid nature of the DAMI cell population. Finally, immunoprecipitation/western blot analysis using a well-characterized human anti-P1^{A2} maternal alloantiserum demonstrated that at least a subpopulation of GPIIIa molecules from clone #24 now expresses the Pro33, P1^{A2} alloantigenic epitope (FIG. 2E).

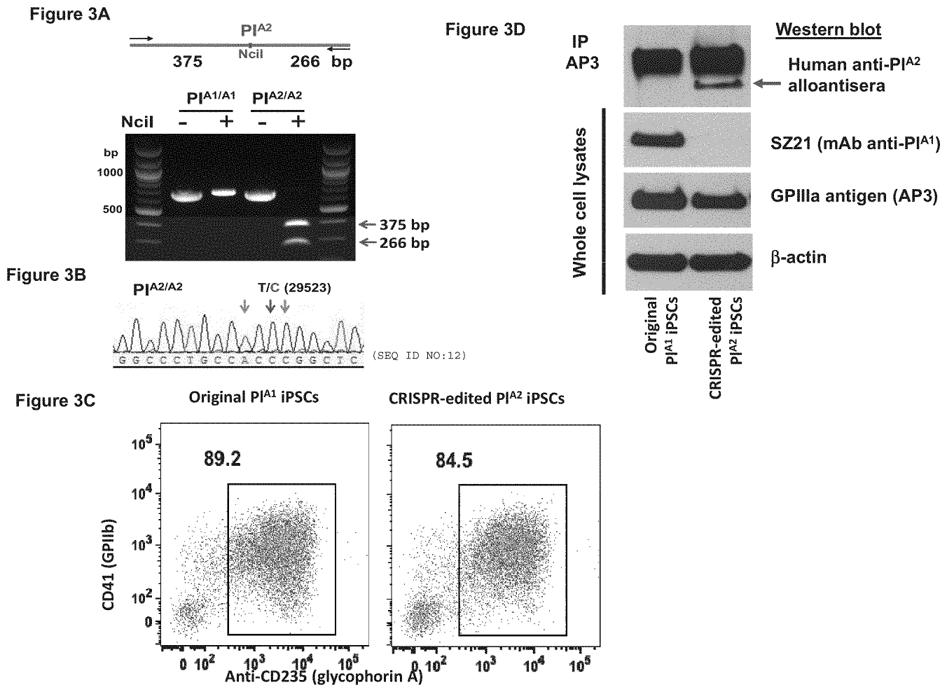

PI^{A1} to P1^{A2} conversion of human iPS cells. Having optimized the conditions for editing the ITGB3 locus in DAMI cells, we applied a similar protocol to edit iPS.K3 cells—a footprint-free cell line that was reprogrammed from human foreskin fibroblasts with non-episomal plasmids (24). DNA sequencing (not shown) of genomic DNA of iPS.K3 cells in and around the P1^{A} polymorphism showed them to be homozygous for the PI^{A1} allele. gRNAs 1 and 2 were cloned into the CRISPR/Cas9 vector, px462, which expresses a puromycin resistance gene (FIG. 1A) and cotransfected with the P1 HDR template into iPS.K3 cells using Nucleofection. Clones from puromycin-resistant colonies were manually picked and expanded two weeks postplating and subjected to diagnostic NciI restriction enzyme digestion to identify clones in which biallelic conversion of PI^{A1} to P1^{A2} had taken place. FIG. 3A shows the NciI digestion pattern of one such homozygous P1 clone, the T>C 29523 genotype of which was verified by DNA sequencing (FIG. 3B).

Wild-type P1A1 homozygous iPS.K3 cells and their CRISPR-edited progeny were then differentiated into hematopoietic progenitor cells (HPCs) using a previously-described serum-free, feeder-free, adherent differentiation system (25, 26). The HPCs generated with this method possess erythroid, megakaryocyte, and myeloid multi-lineage potential, and co-express the CD41/CD61 GPIIb-IIIa complex, as well as CD235 (glycophorin A). As shown in FIG. 3C, HPCs from both iPS cell lines express similar levels of CD41+ and CD235+ on their surface, demonstrating importantly that the CRISPR-modified cells retained full ability to differentiate. Finally, GPIIIa from the P1^{A2}, but not wild-type, iPS cell line expressed the P1^{A2} allelic isoform of GPIIIa, as shown by its specific reactivity with a human anti-P1^{A2} alloantiserum, and its concomitant loss of SZ21 binding (FIG. 3D). Taken together, these data demonstrate successful CRISPR-mediated homozygous conversion of P1^{A1} to P1^{A2} human iPS cells and their subsequent differentiation into GPIIb-IIIa-expressing HPCs.

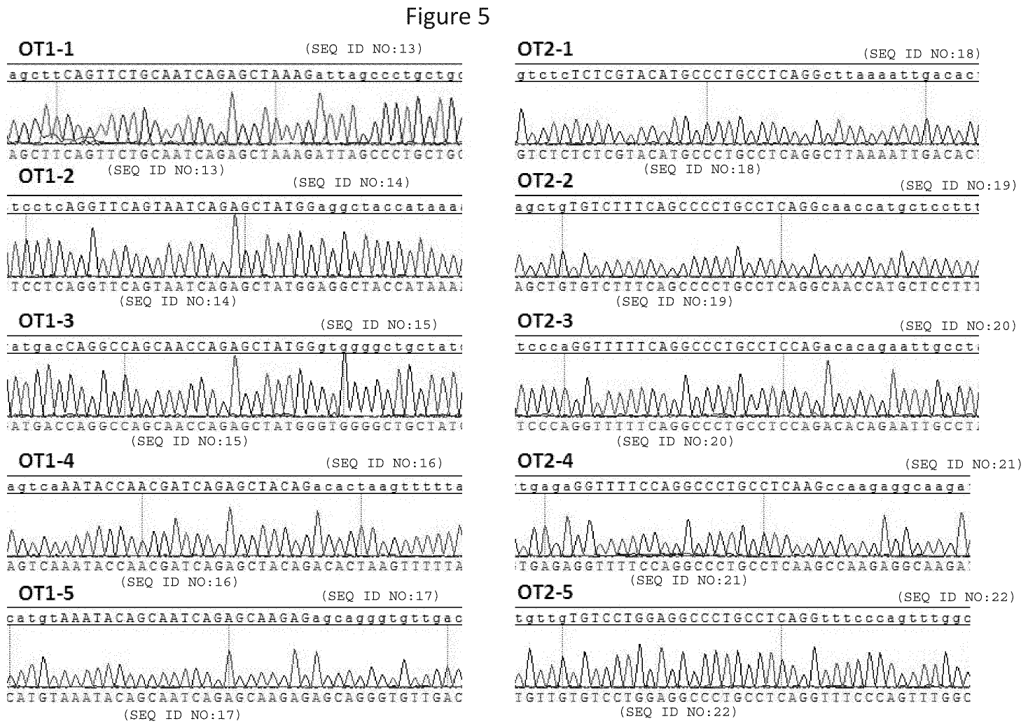

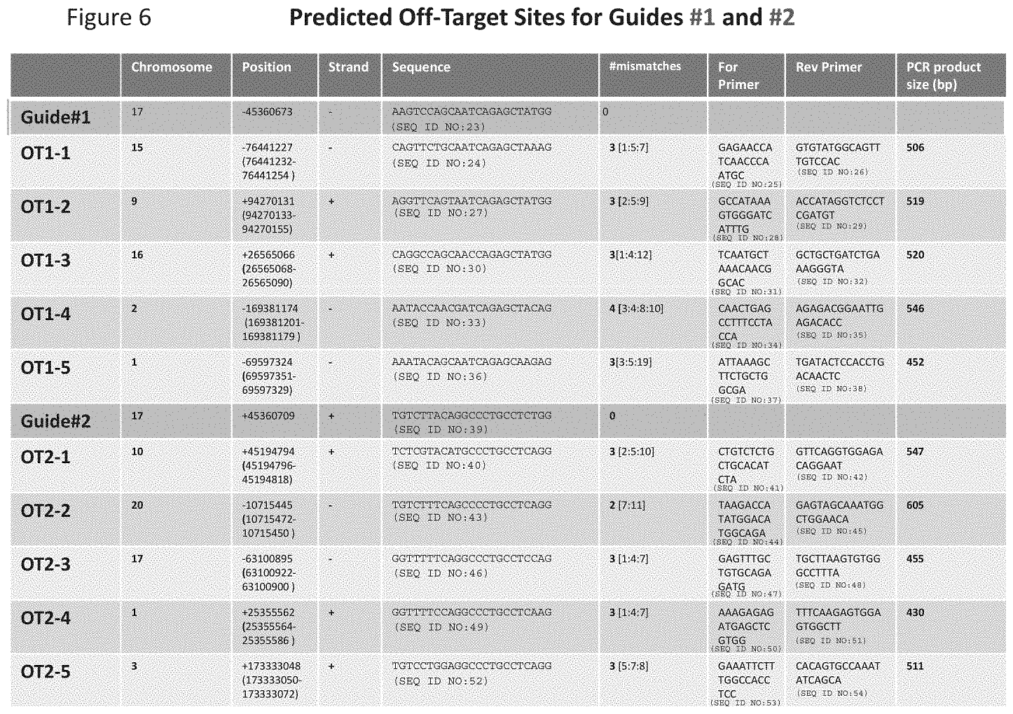

An unintended consequence of CRISPR/Cas9 technology is the occasional introduction of off-target mutations elsewhere in the genome that may affect cell growth and differentiation. This problem can be mitigated in part by using a single-strand Cas9 nickase in combination with two different gRNAs that target opposite strands surrounding the sequence to be edited (FIG. 1B). To evaluate putative off-target effects of the pair of the guide sequences used in this study, we PCR-amplified the top five off-target sites predicted (http://crispr.mit.edu/) for each of our guide sequences (FIG. 6) in our P1^{A2} iPS.K3 cell line, but found no mutations at these loci (FIG. 5).

Discussion

Despite the availability of genotyping for platelet-specific alloantigens, platelet immuno-diagnostics continues to be hampered by the technical complexities of HPA antibody detection—still the gold standard in making a clinical diagnosis of NAIT. Though the majority of human platelet alloantigenic determinants have now been characterized, platelets expressing them are often unavailable, and their detection is additionally hampered by instability or loss of the epitopes following detergent solubilization and storage (27). Finally, serological typing is complicated by the fact that ˜25% of multiparous women produce antibodies against Class I Human Leukocyte Antigens (HLA) (28) that mask detection of platelet-specific alloantigenic epitopes. Taken together, laboratories charged with resolving difficult cases of NAIT have struggled to translate basic scientific discoveries into improved clinical care of families afflicted by this serious condition. The goal of the present investigation, therefore, was to exploit the convergence of CRISPR/Cas9 gene editing and iPS cell 4 platelet technologies to create human platelet progenitors expressing low-frequency platelet alloantigens for diagnostic, investigative, and perhaps future therapeutic, use.

In 2007, the Yamanaka (29, 30) and Thomson (31) labs reported that adult human fibroblasts can be reprogrammed, using a limited number of transcription factors, into pluripotent stem cells. Building upon this discovery, several groups have developed efficient protocols for differentiating iPS cells to HPCs (32, 33), that can be expanded to megakaryocytes (34, 35), and platelets (36-38). While still a long way off from producing a transfusable number of platelets, the ability to generate and cryopreserve iPS cell-derived megakaryocyte progenitor cells leaves open the possibility of maintaining an inexhaustible source of platelets and their progenitors for diagnostic and investigative applications. We sought to exploit this capability to produce antigenically-distinct megakaryocytes and progenitor cells from genetically-customized iPS cells in sufficient quantities for characterization of their platelet-specific alloantigen expression and function by flow cytometry and other diagnostic methods.

Originally discovered as an ancient form of adaptive immunity that functions by incorporating short pieces of DNA into a series of clustered, regularly interspaced short palindromic repeats within the genomes of bacteria and archaea to direct degradation of foreign DNA (12), the CRISPR system of RNA-guided nucleases has largely supplanted earlier zinc finger and TALEN protein-guided nucleases as the preferred gene-editing tool (40). By incorporating a carefully-designed gRNA sequence into a plasmid or lentiviral vector encoding a Cas nuclease, one can engineer double- (41) or single- (42) strand breaks at precise endogenous loci within the genome of almost any cell that can be transfected or transduced, including iPS cells (43), embryonic stem cells, and zygotes (44).

In the present investigation, we combined these technologies to generate iPS cell-derived HPCs that express allele-specific forms of clinically-important human platelet alloantigens. Because it is the most frequent cause of NAIT and PTP in the western world, we performed proof-of-concept genetic manipulations on the P1^{A} alloantigen system, and were able to successfully generate sufficient quantities of PI^{A1}- and P1^{A2}-specific HPCs to perform flow cytometric detection of these human platelet alloantigens—an assay that requires less than ten microliters of human serum. Intact human cells are normally not used for alloantibody detection because maternal sera containing platelet antigen-specific alloantibodies also often contain antibodies specific for Class I HLA that are present on the platelet surface (45, 46). For this reason, time-consuming and technically-demanding antigen-capture ELISA assays are necessary that require hundreds of microliters of maternal alloantisera. HLA detection can be circumvented by introducing a stop codon into the β₂ microglobulin (β2M) gene that encodes the light chain of Class I HLA molecules, which is required for trafficking of all Class I heavy chains to the cell surface (47).

This tactic has been achieved using both siRNA technology in CD34+ hematopoietic stem cells (48) and TALEN technology in iPS cells (37) to produce HLA Class I-deficient platelets, and we have recently employed CRISPR technology to generate a (32M-negative founder iPS line (not shown) into which we plan to introduce polymorphisms that define each of the major human platelet alloantigens. The availability of a potentially replenishable source of alloantigen-specific megakaryocyte and platelet progenitors should go a long way towards improving the diagnosis, treatment and care of patients suffering from this all-to-common cause of morbidity and mortality in newborns.

Methods

Guide RNA plasmid constructs. gRNAs were designed using the CRISPR Design Tool (http://crispr.mit.edu/) to minimize off-target effects and selected to precede a 5′-NGG protospacer-adjacent motif (PAM). gRNAs used in this study were: gRNA1: 5′-AAGTCCAGCAATCAGAGCTA-3′ (SEQ ID NO:1), gRNA2: 5′-TGTCTTACAGGCCCTGCCTC3′ (SEQ ID NO:2). Oligos were annealed and cloned into the BbsI site of the Cas9 expression plasmids px461 or px462 (Addgene, Cambridge, Mass.).

Single-stranded homology-directed repair (HDR) template. A single-stranded oligo-deoxynucleotide (ssODN), 181 nucleotides in length, having the sequence 5′-ACTCGGGCCTCACTCACTGGGAACTCGATGGATTCTGGGGCACAGTTATCCTTCAGCAGATT CTCCTTCAGGTCACAGCGAGGTGAGCCGGGTGGCAGGGCCTGTAAGACAGGAGCCCAAAGA GAAGTCCAGCAATCAGAGCTATGCCGACTCTCTACCTCCTGCAGGCCCTACCACTTCC-3′ (SEQ ID NO:3) was synthesized by Integrated DNA Technologies (IDT, Coralville, Iowa). This oligo corresponds to the antisense strand, and in addition to containing the CTG→CCG P1^{A1} to P1^{A2} substitution, also contains silent mutations within the recognition sequence and the PAM sequence, of gRNA2 to avoid repetitive digestions by Cas9n.

Cell lines and transfection. 2×10⁶ DAMI cells were cultured at 37° C. in 5% CO2 in Iscove's Modified Dulbecco's Medium (IMDM) with 10% horse serum, penicillin (100 U/ml) and streptomycin (100 μg/ml) and transfected with 1 μg of each guide plasmid and 40 pmol of the ssODN HDR template using the Amaxa cell line Nucleofector Kit C (Lonza, Allendale, N.J.) and Nucleofector Program X-005. Transfection efficiency was assessed by visualizing GFP expression using fluorescence microscopy.

Human iPS.K3 cells (24) (kind gift of Dr. Steven Duncan, Medical College of Wisconsin) were grown on StemAdhere Defined Matrix-coated plates (Stemcell Technologies, Vancouver, BC) in mTeSR1/MEF conditioned medium (50:50) containing 4 ng/ml bFGF (Thermo Fisher Scientific, Grand Island, N.Y.) at 37° C. in 4% O2/5% CO2. After incubation with 10 μM ROCK inhibitor Y27632 (StemRD Inc. Burlingame, Calif.), 2×105 cells were transfected with 0.5 μg of each guide plasmid and 40 pmol of the HDR oligonucleotide using the Amaxa P3 primary cell 4D Nucleofector Kit (Lonza) and Nucleofector Program CB-150. The cells were then plated on DR4 MEF feeder cells supplemented with 10 μM Y27632. 24-hour post-transfection puromycin was applied at a concentration of 1 μg/ml for 24 hr. Single clones were harvested at 12 to 14 days post-puromycin-selection and re-plated on StemAdhere-coated plates. Karyotyping of the iPSC lines was performed by Dynacare Laboratories (Milwaukee, Wis.) after genotyping to identify the correct lines and every 15 passages routinely during culture.

Differentiation of iPS.K3 cells. Wild-type iPS.K3 cells and CRISPR-edited P1 iPS.K3 cells were differentiated to HPCs as previously described (25, 26). Briefly, cells were cultured in feeder-free conditions prior to plating on Matrigel for differentiation. The medium and cytokine changes were followed as described with the following modification. The GSK-3β inhibitor, CHIR99021 (Cayman, Ann Arbor, Mich.) (0.5-1 μM) was used instead of Wnt3a. Cells were cultured at 37° C., 5% CO₂, 5% O₂ and 90% N₂ for 7-9 days and loosely adherent HPCs were collected by carefully removing the supernatant. Cells were analyzed by flow cytometry for the surface expression of CD41a and CD235a.

Flow cytometry. 24 hrs post-transfection, DAMI cells were washed and resuspended in growth medium containing 25 mM HEPES buffer and filtered through 100 μm MACS SmartStrainers (Miltenyi Biotec, San Diego, Calif.). GFP⁺ cells were analyzed with a BD Biosciences (San Jose, Calif.) ARIA-IIIu Cell Sorter. Non-transfected cells were used as negative control. GFP⁺ cells were sorted as single cells into individual wells of 96-well plates. Analysis of iPSC-derived HPCs was performed using a CANTO Flow Cytometer (Becton Dickinson, San Jose, Calif.). The antibodies used were anti-CD235-APC and CD41a-PE (BD Biosciences). Flow cytometry data were analyzed using FLOWJO software (Tree Star Inc., Ashland, Oreg.).

Detection of introduced mutations in genomic DNA. Cells were harvested 72 hrs after transfection, and DNA was extracted using a QIAamp DNA mini kit (Qiagen, Germantown, Md.) according to the manufacture's protocol. The genomic region flanking the P1^{A1} site was amplified using PCR primer GPIIIa fw2: 5′-CGTGGAATTCGCTGGTCTACCAGGCATCTT-3′ (SEQ ID NO:4) and GPIIIa rev2: 5′-CCGAAGCTTACCTTGTGCTCTATGCCCAC-3′ (SEQ ID NO:5). PCR products were purified using QIAquick Spin Column (QIAGEN). Purified PCR products (400 ng) were mixed with 1× Taq DNA polymerase PCR buffer, denatured at 95° C. and reannealed to form DNA heteroduplexes. The reannealed PCR products were treated with Surveyor nuclease (IDT) following the manufacturer's protocol and analyzed on a 2% agarose gel. Quantification was based on relative band intensities. The percentage of DNA mismatches was determined by the formula 100×{1−[1−(b+c)/(a+b+c)]1/2}, wherein a is the integrated intensity of the undigested PCR product and b and c are the integrated intensities of each cleavage product.

Genotyping. Genomic DNA was extracted from each clone of DAMI and iPS.K3 cells using the QUICKEXTRACT DNA Extraction Solution (Epicenter, Madison, Wis.) following the manufacture's protocol. The region surrounding the P1^{A1}/P1^{A2} in polymorphism was amplified using GPIIIa fw1: 5′-CGTGGAATTCGGCATCTTACTGTACAGGCT-3′ (SEQ ID NO:6) and GPIIIa rev1: 5′-GGCAAGCTTA-AGACTTCCTCCTCAGACCT-3′ (SEQ ID NO:7). PCR products were purified using QIAquick Spin Column, digested with NciI (New England Biolabs Inc., Ipswich, Mass.), and analyzed on 2% agarose gels.

Immunoprecipitation and Western blot analysis. 2×10⁷ DAMI cells or 3×10⁶ iPSC-derived HPCs were lysed in 20 mM Tris (pH7.4), 150 mM NaCl, 1% Triton X-100, 1 mM EDTA, 10 mM N-ethylmaleimide and protease inhibitor cocktail (Thermo Fisher Scientific). Lysates were centrifuged at 17,000×g for 15 min at 4° C. Supernatants were collected, precleared with protein G sepharose and then incubated with the anti-GPIIIa monoclonal antibody (mAb) AP3 overnight at 4° C. Immune complexes were collected on protein G sepharose beads, eluted with nonreducing SDS sample buffer, and loaded onto 4-20% polyacrylamide gels. Following electrophoresis, the samples were electrotransferred onto PVDF membrane (EMD Millipore, Billerica, Mass.) and immunoblotted with either human anti-P1^{A2} antisera, the P1^{A1}-selective murine mAb, SZ21 (Beckman Coulter, Brea, Calif.), AP3, or a mouse mAb specific for β-actin (Sigma, St. Louis, Mo.). Bound antibodies were visualized using species-specific peroxidase-conjugated donkey anti-human IgG (H+L) or goat anti-mouse IgG (H+L) secondary antibodies from Jackson ImmunoResearch Laboratories (West Grove, Pa.).

### REFERENCES

1. Aster R H. Studies of the fate of platelets in rats and man. Blood. 1969; 34(2):117-28.
2. Newman P J, McFarland J G, and Aster R H. In: Loscalzo J, and Schafer A I eds. Thrombosis and Hemorrhage. Lippincott Williams and Wilkins; 2003:441-56.
3. Williamson L M, Hackett G, Rennie J, Palmer C R, Maciver C, Hadfield R, Hughes D, Jobson S, and Ouwehand W H. The natural history of fetomaternal alloimmunization to the platelet-specific antigen HPA-1a (PIA1, Zwa) as determined by antenatal screening. Blood. 1998; 92(7):2280-7.
4. Kjeldsen-Kragh J, Killie M K, Tomter G, Golebiowska E, Randen I, Hauge R, Aune B, Oian P, Dahl L B, Pirhonen J, et al. A screening and intervention program aimed to reduce mortality and serious morbidity associated with severe neonatal alloimmune thrombocytopenia. Blood. 2007; 110(3):833-9.
5. Giovangrandi Y, Daffos F, Kaplan C, Forestier F, Mac A J, and Moirot M. Very early intracranial haemorrhage in alloimmune fetal thrombocytopenia. Lancet. 1990; 336(8710):310.
6. Bonacossa I A, and Jocelyn L J. Alloimmune thrombocytopenia of the newborn: neurodevelopmental sequelae. Am J Perinatol. 1996; 13(4):211-5.
7. Dreyfus M, Kaplan C, Verdy E, Schlegel N, Durand-Zaleski I, and Tchernia G. Frequency of immune thrombocytopenia in newborns: a prospective study Immune Thrombocytopenia Working Group. Blood. 1997; 89(12):4402-6.
8. Spencer J A, and Burrows R F. Feto-maternal alloimmune thrombocytopenia: a literature review and statistical analysis. The Australian & New Zealand journal of obstetrics & gynaecology. 2001; 41(1):45-55.
9. Turner M L, Bessos H, Fagge T, Harkness M, Rentoul F, Seymour J, Wilson D, Gray I, Ahya R, Cairns J, et al. Prospective epidemiologic study of the outcome and cost-effectiveness of antenatal screening to detect neonatal alloimmune thrombocytopenia due to anti-HPA-1a. Transfusion. 2005; 45(12):1945-56.
10. Bussel J. Diagnosis and management of the fetus and neonate with alloimmune thrombocytopenia. Journal of thrombosis and haemostasis: JTH. 2009; 7 (Suppl 1):253-7.
11. van Loghem J J, Dorfmeyer H, van der Hart H, and Schreuder F. Serological and genetical studies on a platelet antigen (Zw). Vox Sang. 1959; 4(2):161-9.
12. Shulman N R, Aster R H, Pearson H A, and Hiller M C. Immunoreactions involving platelets. V I. Reactions of maternal isoantibodies responsible for neonatal purpura. Differentiation of a second platelet antigen system. The Journal of clinical investigation. 1962; 41(5):1059-69.
13. Newman P J, Derbes R S, and Aster R H. The human platelet alloantigens, PI^{A1} and P1^{A2}, are associated with a leucine33/proline33 amino acid polymorphism in membrane glycoprotein Ma, and are distinguishable by DNA typing. JClinInvest. 1989; 83(5):1778-81.
14. Metcalfe P, Watkins N A, Ouwehand W H, Kaplan C, Newman P, Kekomaki R, De Haas M, Aster R, Shibata Y, Smith J, et al. Nomenclature of human platelet antigens. Vox Sang. 2003; 85(3):240-5.
15. McFarland J G, Aster R H, Bussel J B, Gianopoulos J G, Derbes R S, and Newman P J. Prenatal diagnosis of neonatal alloimmune thrombocytopenia using allele-specific oligonucleotide probes. Blood. 1991; 78(9):2276-82.
16. Curtis B R. Genotyping for human platelet alloantigen polymorphisms: applications in the diagnosis of alloimmune platelet disorders. Seminars in thrombosis and hemostasis. 2008; 34(6):539-48.
17. Le Toriellec E, Chenet C, and Kaplan C. Safe fetal platelet genotyping: new developments. Transfusion. 2013; 53(8):1755-62.
18. An Q X, Li C Y, Xu L J, Zhang X Q, Bai Y J, Shao Z J, and Zhang W. High-throughput simultaneous genotyping of human platelet antigen-1 to -16 by using suspension array. Transfusion. 2013; 53(11):2722-8.
19. Skogen B, Bellissimo D, Hessner M J, Santoso S, Aster R H, Newman P J, and McFarland J G. Rapid determination of platelet alloantigen genotypes by polymerase chain reaction using allele specific primers. Transfusion. 1994; 34(11):955-60.
20. Vassallo R R, Jr. New paradigms in the management of alloimmune refractoriness to platelet transfusions. Current opinion in hematology. 2007; 14(6):655-63.
21. Bussel J B, and Sola-Visner M. Current approaches to the evaluation and management of the fetus and neonate with immune thrombocytopenia. Seminars in perinatology. 2009; 33(1):35-42.
22. Pacheco L D, Berkowitz R L, Moise K J, Jr., Bussel J B, McFarland J G, and Saade G R. Fetal and neonatal alloimmune thrombocytopenia: a management algorithm based on risk stratification. Obstetrics and gynecology. 2011; 118(5):1157-63.
23. Wilcox D A, Olsen J C, Ishizawa L, Griffith M, and White G C, 2nd. Integrin αIIb promoter-targeted expression of gene products in megakaryocytes derived from retrovirus-transduced human hematopoietic cells. Proceedings of the National Academy of Sciences of the United States of America. 1999; 96(17):9654-9.
24. Si-Tayeb K, Noto F K, Sepac A, Sedlic F, Bosnjak Z J, Lough J W, and Duncan S A. Generation of human induced pluripotent stem cells by simple transient transfection of plasmid DNA encoding reprogramming factors. BMC developmental biology. 2010; 10:81.
25. Mills J A, Paluru P, Weiss M J, Gadue P, and French D L. In: Qu KDBaC-K ed. Methods in Molecular Biology. New York: Springer Science+Business Media; 2014:181-94.
26. Paluru P, Hudock K M, Cheng X, Mills J A, Ying L, Galvao A M, Lu L, Tiyaboonchai A, Sim X, Sullivan S K, et al. The negative impact of Wnt signaling on megakaryocyte and primitive erythroid progenitors derived from human embryonic stem cells. Stem cell research. 2014; 12(2):441-51.
27. Harrison C R, Curtis B R, McFarland J G, Huff R W, and Aster R H. Severe neonatal alloimmune thrombocytopenia caused by antibodies to human platelet antigen 3a (Baka) detectable only in whole platelet assays. Transfusion. 2003; 43(10):1398-402.
28. Ouwehand W H. The dilemma of screening for antibodies against low-frequency human platelet antigens. Transfusion. 2005; 45(3):288-9.
29. Takahashi K, Tanabe K, Ohnuki M, Narita M, Ichisaka T, Tomoda K, and Yamanaka S. Induction of pluripotent stem cells from adult human fibroblasts by defined factors. Cell. 2007; 131(5):861-72.
30. Ohnuki M, Takahashi K, and Yamanaka S. Generation and characterization of human induced pluripotent stem cells. Current protocols in stem cell biology. 2009; Chapter 4:Unit 4A 2.
31. Yu J, Vodyanik M A, Smuga-Otto K, Antosiewicz-Bourget J, Frane J L, Tian S, Nie J, Jonsdottir G A, Ruotti V, Stewart R, et al. Induced pluripotent stem cell lines derived from human somatic cells. Science. 2007; 318(5858):1917-20.
32. Choi K D, Vodyanik M A, and Slukvin, I I. Generation of mature human myelomonocytic cells through expansion and differentiation of pluripotent stem cell-derived lin-CD34+CD43+CD45+ progenitors. The Journal of clinical investigation. 2009; 119(9):2818-29.
33. Kennedy M, D'Souza S L, Lynch-Kattman M, Schwantz S, and Keller G. Development of the hemangioblast defines the onset of hematopoiesis in human E S cell differentiation cultures. Blood. 2007; 109(7):2679-87.
34. Sullivan S K, Mills J A, Koukouritaki S B, Vo K K, Lyde R B, Paluru P, Zhao G, Zhai L, Sullivan L M, Wang Y, et al. High-level transgene expression in induced pluripotent stem cell-derived megakaryocytes: correction of Glanzmann thrombasthenia. Blood. 2014; 123(5):753-7.
35. Nakamura S, Takayama N, Hirata S, Seo H, Endo H, Ochi K, Fujita K, Koike T, Harimoto K, Dohda T, et al. Expandable megakaryocyte cell lines enable clinically applicable generation of platelets from human induced pluripotent stem cells. Cell stem cell. 2014; 14(4):535-48.
36. Thon J N, Mazutis L, Wu S, Sylman J L, Ehrlicher A, Machlus K R, Feng Q, Lu S, Lanza R, Neeves K B, et al. Platelet bioreactor-on-a-chip. Blood. 2014; 124 (12):1857-67
37. Feng Q, Shabrani N, Thon J N, Huo H, Thiel A, Machlus K R, Kim K, Brooks J, Li F, Luo C, et al. Scalable generation of universal platelets from human induced pluripotent stem cells. Stem Cell Reports. 2014; 3(5):817-31.
38. Wang Y, Hayes V, Jarocha D, Sim X, Harper D C, Fuentes R, Sullivan S K, Gadue P, Chou S T, Torok-Storb B J, et al. Comparative analysis of human ex vivo-generated platelets vs megakaryocyte-generated platelets in mice: a cautionary tale. Blood. 2015; 125(23):3627-36.
39. Karginov F V, and Hannon G J. The CRISPR system: small RNA-guided defense in bacteria and archaea. Molecular cell. 2010; 37(1):7-19.
40. Mali P, Esvelt K M, and Church G M. Cas9 as a versatile tool for engineering biology. Nature methods. 2013; 10(10):957-63.
41. Cong L, Ran F A, Cox D, Lin S, Barretto R, Habib N, Hsu P D, Wu X, Jiang W, Marraffini L A, et al. Multiplex genome engineering using CRISPR/Cas systems. Science. 2013; 339(6121):819-23.
42. Ran F A, Hsu P D, Wright J, Agarwala V, Scott D A, and Zhang F. Genome engineering using the CRISPR-Cas9 system. Nature protocols. 2013; 8(11):2281-308.
43. Ding Q, Regan S N, Xia Y, Oostrom L A, Cowan C A, and Musunuru K. Enhanced efficiency of human pluripotent stem cell genome editing through replacing TALENs with CRISPRs. Cell stem cell. 2013; 12(4):393-4.
44. Wang H, Yang H, Shivalila C S, Dawlaty M M, Cheng A W, Zhang F, and Jaenisch R. One-step generation of mice carrying mutations in multiple genes by CRISPR/Cas-mediated genome engineering. Cell. 2013; 153(4):910-8.
45. Nielsen L S, and Svejgaard A. H L-A immunization and H L-A types in pregnancy. Tissue Antigens. 1972; 2(4):316-27.
46. Aster R H, Szatkowski N, Liebert M, and Duquesnoy R J. Expression of HLA-B12, HLA-B8, w4, and w5 on platetelets. TransplantProc. 1977; 9(4):1695-6.
47. Lancet D, Parham P, and Strominger J L. Heavy chain of HLA-A and HLA-B antigens is conformationally labile: a possible role for b2-microglobulin. Proceedings of the National Academy of Sciences of the United States of America. 1979; 76(8):3844-8.
48. Gras C, Schulze K, Goudeva L, Guzman C A, Blasczyk R, and Figueiredo C. HLA-universal platelet transfusions prevent platelet refractoriness in a mouse model. Human gene therapy. 2013; 24(12):1018-28.
49. Ran F A, Hsu P D, Lin C Y, Gootenberg J S, Konermann S, Trevino A E, Scott D A, Inoue A, Matoba S, Zhang Y, et al. Double nicking by RNA-guided CRISPR Cas9 for enhanced genome editing specificity. Cell. 2013; 154(6):1380-9.
50. Shen B, Zhang W, Zhang J, Zhou J, Wang J, Chen L, Wang L, Hodgkins A, Iyer V, Huang X, et al. Efficient genome modification by CRISPR-Cas9 nickase with minimal off-target effects. Nature methods. 2014; 11(4):399-402.

### Example 2

Human platelet alloantigens (HPAs) reside on functionally important platelet membrane glycoproteins. Currently, 6 biallelic HPA systems (HPA-1, -2, -3, -4, -5, and -15) as well as 26 single low-frequency antigens have been described on 6 platelet glycoproteins.¹⁻⁴ Almost all the HPAs are caused by single amino acid substitutions encoded by single nucleotide polymorphisms on 1 of 6 different platelet glycoproteins. The only exception, HPA-14b, is caused by a single amino acid deletion resulting from an in-frame triplet deletion in the ITGB3 gene rather than a single nucleotide polymorphism.⁵

Antibodies that form against HPAs are responsible for several clinically important alloimmune bleeding disorders, including fetal and neonatal alloimmune thrombocytopenia (FNAIT, variously referred to in the literature as NATP, NAIT, and FNIT), posttransfusion purpura, and platelet transfusion refractoriness.^{6,7} In these conditions, serologic detection and characterization of anti-HPA alloantibodies are essential for proper diagnosis, treatment and, in FNAIT, the prevention of severe thrombocytopenia and its bleeding risks in subsequent pregnancies. Over the years, many creative assays have been developed for detecting alloantibodies against HPAs in patient sera, and they can be grouped into 2 categories: whole-platelet methods and glycoprotein-specific methods.⁸ That human platelets also express class I HLA antigens on their surface, coupled with the fact that patient HPA antisera often contain HLA class I antibodies (the binding of which can mask the presence of HPA-specific antibodies), creates a significant problem for whole-platelet antibody detection methods, thus limiting the HPA alloantibody detection to glycoprotein-specific assays for these patients. Currently, antigen capture assays based on the use of a monoclonal antibody such as modified antigen capture enzyme-linked immunosorbent assay (MACE)⁹ and monoclonal antibody immobilization of platelet antigens (MAIPA)′° are the most popular glycoprotein-specific assays in use because of their high specificity and sensitivity.

However, these methods can be performed only by specialized laboratories, are tedious, and require solubilization of platelet glycoproteins with detergent, a procedure that can result in the loss of labile antigenic determinants.^{11,12} In addition, the monoclonal antibodies used to capture the glycoproteins sometimes compete with the binding of the alloantibodies present in patient sera, resulting in false-negative results.⁸

HPA-3, historically known as the Bak^{a}/Bak^{b} alloantigen system, was first described in 1980 by von dem Borne et al” as a new specificity present in a maternal alloantibody in a case of FNAIT. The antigen was localized to GPIIb in 1986′⁴ and was found to be the result of an Ile843Ser polymorphism near the carboxyl terminus (C terminus) of the GPIIb extracellular domain.¹⁵ Interestingly, amino acid 843 is only 6 amino acids away from a Va1837Met polymorphism, identified as the HPA-9b antigen (also known as Max®) in an FNAIT case.¹⁶ HPA-3 antibodies are relatively rare, but they can often induce severe FNAIT with intracranial hemorrhage.^{11,17-20} Several studies have identified anti-HPA-3 antibodies as the most problematic specificity to detect,^{11,12,17,20,21} likely because of the heterogeneity of HPA-3 epitope formation. In addition to the nearby Ile843Ser polymorphism, some HPA-3 epitopes depend upon the 3-dimensional conformation of GPIIb and are thus sensitive to fixation or detergents.^{11,12,17,20} Others require as part of their recognition epitope adjacent O-linked carbohydrate structures or terminal sialic acids that can be lost during platelet storage.²¹⁻²³ One biochemical study identified Ser847 as a major O-glycosylation site required for anti-HPA-3a antibody binding.²⁴

In contrast to the HPA-3 alloantigen system, HPA-9b is a low-frequency HPA located near the C terminus of the extracellular domain of GPIIb, with an estimated gene frequency of 0.002 to 0.003 in whites.^{16,25} Despite its low frequency, FNAIT caused by anti-HPA-9b alloantibodies is not uncommon, and nearly all cases are accompanied by severe thrombocytopenia and bleeding.^{25,26} Detection of anti-HPA-9b alloantibodies can be extremely difficult using existing serologic assays,²⁵⁻²⁷ probably because of the same complications encountered for detection of alloantibodies to the nearby HPA-3 polymorphism.

Induced pluripotent stem cells (iPSCs) have become an optimal source for large-scale in vitro megakaryocyte (MK) and platelet production because they offer the advantages of unlimited expansion in culture and are amenable to genetic manipulation.^{28,29} To overcome the limitations for current HPA alloantibody detection, we generated a series of genetically edited iPSC lines lacking HLA class I antigen expression and that display, upon differentiation into human MKs, homozygous forms of GPIIb that carry the HPA-3a, HPA-3b, or HPA-9b alloantigens. These iPSC-derived MKs are suitable for simple, 1-step flow cytometric detection of anti-HPA-3a, HPA-3b, and HPA-9b alloantibodies in patient sera without HLA class I antibody interference. Because these cell lines share an identical genetic background (except for the genetically targeted HPA-3a, HPA-3b, and HPA-9b isoforms of GPIIb), they offer extremely high specificity for diagnostic purposes compared with donor-derived platelets. Ready availability of MKs that express on the cell surface intact glycoproteins that carry carbohydrate moieties that likely mimic those found on human platelets should facilitate detection of HPA alloantibodies that are normally difficult or impossible to detect using existing techniques.

### Materials and Methods

Anti-HPA-3a (patient 1 [P1]-P3) and anti-HPA-9b (P1-P2) patient sera were from the Platelet and Neutrophil Immunology Laboratory (Versiti, Milwaukee, Wis.). All these samples, except anti-HPA-3a (P3), were positive in a MACE assay. The anti-HPA-3a (P3) sample tested negative in the MACE assay but was positive using the platelet suspension immune fluorescence test. Anti-HPA-3b (P1-P3) and anti-HPA-9b (P3) patient sera were from the Institute for Clinical Immunology and Transfusion Medicine (Giessen, Germany). These samples were all positive in an MAIPA assay. Anti-HPA-9b (P4-P6) suspected sera were provided by Richard Aster.

Guide RNAs (gRNAs) were designed using the CRISPR Design Tool (crispr.mit.edu/and benchling.com/crispr) to minimize off-target effects, and they were selected to precede a 5′-NGG protospacer-adjacent motif (PAM). gRNA sequences are listed below. Oligos were annealed and cloned into the BbsI site of the Cas9 expression plasmids PX459 V2.0 (Addgene, Cambridge, Mass.).

gRNA Sequences

| | 5′-3′sequence |
|---|---|
| Guide targeting β2M | GAGTAGCGCGAGCACAGCTA |
| | (SEQ ID NO: 58) |
| Guide 1 for | CGGCCCCAGACCAACCACCG |
| generating HPA-3b | (SEQ ID NO: 59) |
| Guide 2 for | AGCACTTCAAGTGAACATGG |
| generating HPA-3b | (SEQ ID NO: 60) |
| Guide for | GGGCAGCCCCCAGTCCACCT |
| generating HPA-9b | (SEQ ID NO: 61) |

To construct the donor plasmid for HPA-3b, a 1.6-kb ITGA2B gBlock Gene Fragment was synthesized by Integrated DNA Technologies (Coralville, Iowa). The sequence of the gBlock Gene Fragment is provided in supplemental Data. The fragment contains the T→G (HPA-3a to HPA-3b) substitution as well as silent mutations within the recognition sequence and the PAM sequence of 2 gRNAs to avoid repetitive digestions by Cas9 and also introduces an MfeI site into the genome for genotyping. The recognition sequence and the PAM sequence of guide 2 for generating HPA-3b were added to both ends of the gBlock Gene Fragment for linearizing the donor templates in the transfected cells. The gBlock Gene Fragment was cloned into the pMiniT 2.0 vector using an NEB polymerase chain reaction (PCR) cloning kit (New England Biolabs Inc., Ipswich, Mass.).

HPA-3b gBlock fragment:
(SEQ ID NO: 57)
5′-ggcgtcgaattcAGCACTTCAAGTGAACATGGaggAGCCAGAATCCA
AACAGCAAGATTGTGCTGCTGGACGTGCCGGTCCGGGCAGAGGCCCAAGT
GGAGCTGCGAGGGTGAGAGGCCAGGGGTGGAGAAGGGAGATGGCATTCAG
GGCTCTAAACTCCAGGGGGCGCTGGGGAAACCTCACAGGCCAATCAGGGC
ATCACACTCTCTCTGGGGGTCTTGGGCACCTGCAGGAACTCCTTTCCAGC
CTCCCTGGTGGTGGCAGCAGAAGAAGGTGAGAGGGAGCAGAACAGCTTGG
ACAGCTGGGGACCCAAAGTGGAGCACACCTATGAGGTATTGGGGAGCCTC
GCGTCCCTGGCTGGGGTGAGCGGGTCCTCAGAACTCCGGGTGAGGCGCTA
AGCTCCCCACACCCTGCCACCACCACCCCTTCAGCTCCACAACAATGGCC
CTGGGACTGTGAATGGTCTTCACCTCAGCATCCACCTTCCGGGACAGTCC
CAGCCCTCCGACCTGCTCTACATCCTGGATATACAGCCCCAGGGGGGCCT
TCAGTGCTTCCCACAGCCTCCTGTCAACCCTCTCAAGGTAAGAGCTGGGT
GGAAGAAAGACCTGGGAAGGCGGCCCCAGACCAACCAACGTTGCACCTCT
GTGGGCTGGGGTTCGGGGGAGACCTGGGCCTGACCACTCCTTTGCCCCCC
CAGGTGGACTGGGGGCTGCCCAGCCCCAGCCCCTCCCCCATTCACCCGGC
CCATCACAAGCGGGATCGCAGACAGATCTTCCTGCCAGAGCCCGAGCAGC
CCTCGAGGCTTCAGGATCCAGTTCTCGTAGTGAGCAGGCTCTCTGGTCTC
TGGCCCGGCCTCCCCGGGACCCACGGGGCAGAGGGGATGGGAGGAGGGAG
AGGGGTCCGGGTGTGCTGTGGGCCTCTGTGGGCCACGCTTGGTCCCTGGG
AGCACTTCAATTGCAGTTGGAGTAGCATGCTGGCTTGTGTCTGGGGTGAG
CTGAAAGACACTTGCACTTTTTAAAAGCTTCCCAGTACGTTAAGGAGCAT
AAAACAATGCCAAAGCAAGGTTATCATAGATCTGAGCATTGTGCGCTGGG
GGATGACCCTCCCTGCATCTCTGGGACTATGTGAGCAAGCCCGTGGAAAG
ACAGCATCCGAAGCTTGGATCCAAGGCCCTTCCTGATGGGAAGGCCACCG
CTTCCTGAACCCCCGGCCCCTTCTGCGTTGGGTCCTGGGGGTAAGGGGGT
GGGGGATGATGGGGTGATGGGCCGGGACGGGCTGGGGACTGACGATGCTT
CCCCTCAGAGCTGCGACTCGGCGCCCTGTACTGTGGTGCAGTGTGACCTG
CAGGAGATGGCGCGCGGGCAGCGGGCCATGGTCACGGTGCTGGCCTTCCT
GTGGCTGCCCAGCCTCTACCAGGTGGGGTGGGCCGTGGTGGGGCGGGGCC
GGGCCTTCTGGGCGGGGACCACTTTGCTCTGGGAGGGGCGGGGTTTGGTG
TGGGAGGGCAGGAAGAGAGGGAAGGCAAGGTTTACTTTGGGGGATTGCAG
TGGGATTAGGTCAGAGGCctCCATGTTCACTTGAAGTGCTgaattcgcag
cg-3′

A single-stranded oligo-deoxynucleotide (ssODN) HPA-9b donor template was synthesized by Integrated DNA Technologies. The sequence of the oligo is provided in supplemental Data. This oligo corresponds to the antisense strand, contains a G→A (HPA-9a to HPA-9b) substitution, a silent mutation within the PAM sequence of gRNA to avoid repetitive digestions by Cas9, and introduces a PstI site into the genome for genotyping.

HPA-9b ssODN donor template
(SEQ ID NO: 62)
5′CTCGAGGGCTGCTCGGGCTCTGGCAGGAAGATCTGTCTGCGATCCCGC
TTGTGATGGGCCGGGTGAATGGGAGAGGGGCTGGGGCTGGGCAGCCCCCA
GTCCATCTGCAGGGGCAAAGGAGTGGTCAGGCCCAGGTCTCCCCCGAACC
CCAGCCCACAGAGGTGCCCCGGTGGTTGGTCTGGGGCCGCCTTCCCAGGT
C-3′

Human OT1-1 iPSCs³⁰ were cultured on Matrigel (Corning, Corning, N.Y.)-coated plates in mTeSR1 medium (STEMCELL Technologies Inc., Cambridge, Mass.) at 37° C. in 4% O₂ and 5% CO₂. After incubation with 10 mM Rho kinase (ROCK) inhibitor Y27632 (StemRD Inc., Burlingame, Calif.), 2×10⁵ cells were transfected with 0.5 or 1 mg of each guide plasmid in the presence or absence of 0.5 mg of the HPA-3b plasmid donor or 40 pmol of HPA-9b ssODN donor using the Amaxa P3 primary cell 4D Nucleofector Kit (Lonza, Allendale, N.J.) and Nucleofector Program CB-150. The cells were then plated on Matrigel-coated plates with 10 mM Y27632. Puromycin was applied at 24 hours after transfection at a concentration of 1 mg/mL for 48 hours. Single clones were harvested at 12 to 14 days after puromycin selection and re-plated on Matrigel-coated plates. Karyotyping of the iPSC lines was performed every 15 passages by Wisconsin Diagnostic Laboratories (Milwaukee, Wis.) to verify continued diploidy of all iPSC lines.

Genomic DNA was extracted from each iPSC clone by using the QuickExtract DNA Extraction Solution (Epicenter, Madison, Wis.) following the manufacture's protocol. The region surrounding the HPA-3 and HPA-9 polymorphisms was amplified by PCR using the pair of primers: ITGA2B for 5′-CGTGGAATTCAAGTG GAGCACACCTATGAG-3′ (SEQ ID NO:55) and ITGA2B rev 5′-GGCAAGC-TTACCTTGCTTTGGCATTGTTT-3′ (SEQ ID NO:56). PCR products were purified using QiaQuick Spin Column, digested with MfeI or PstI (New England Biolabs Inc.), and analyzed on 2% agarose gels.

CRISPR-edited iPSC lines were differentiated to hematopoietic progenitor cells (HPCs) as previously described.^{31,32} Briefly, cells were plated on Matrigel for differentiation. Media and cytokine changes were observed as described except that the GSK-3b inhibitor CHIR99021 (1 mM) (Tocris Bioscience, Minneapolis, Minn.) was used instead of Wnt3a. Cells were cultured at 37° C. with 4% 02 and 5% CO2 for 9 days, and loosely adherent HPCs were collected by carefully removing the supernatant. Cells were analyzed by flow cytometry to confirm surface expression of CD41a and CD235a. The HPCs were further differentiated to MKs in serum-free differentiation medium, which is composed of Iscove modified Dulbecco medium (Thermo Fisher Scientific, Waltham, Mass.) containing 25% Ham's F12 (Corning), 0.5% N2 (Thermo Fisher Scientific), 1% B27 without vitamin A (Thermo Fisher Scientific), 0.05% bovine serum albumin (Sigma, St. Louis, Mo.), 2-mM _{L}-glutamine and penicillin/streptomycin supplemented with 50 ng/mL stem cell factor (R&D Systems, Minneapolis, Minn.), and 50 ng/mL thrombopoietin (R&D Systems) at 37° C. in 5% CO₂ for 6 days. MKs were analyzed by flow cytometry to confirm the surface expression of CD41 and CD42b.

3×10⁵ iPSCs were incubated with fluorescein isothiocyanate-conjugated anti-human HLA-A, -B, -C and allophycocyanin-conjugated anti-human β2-microglobulin (β2M) antibodies (BioLegend, San Diego, Calif.) for 20 minutes at room temperature. 3×10⁵ iPSC-derived MKs were incubated with 25 to 50 mL of normal human sera or patient sera for 30 minutes at room temperature. After washing, the cells were incubated with fluorescein isothiocyanate-conjugated anti-CD41, allophycocyanin-conjugated anti-CD42b (BioLegend), and phycoerythrin-conjugated donkey anti-human immunoglobulin G (Jackson ImmunoResearch Laboratories, West Grove, Pa.) at room temperature for 20 minutes. Flow cytometry was performed using a BD LSRII flow cytometer (BD Biosciences). Flow cytometry data were analyzed using FlowJo software (Tree Star Inc., Ashland, Oreg.).

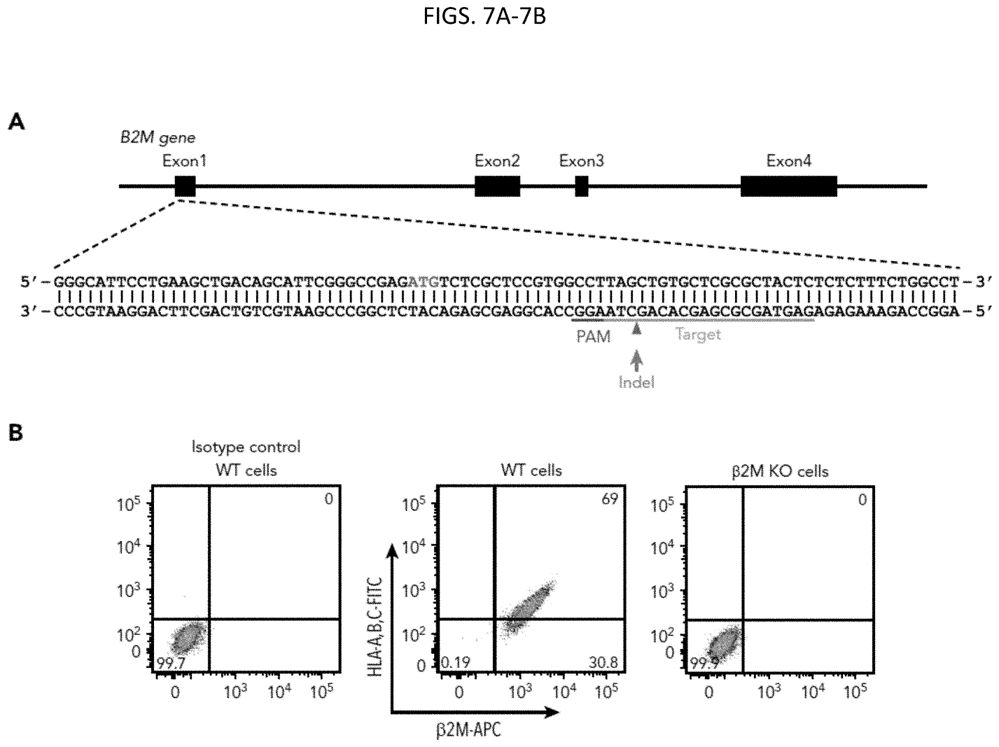

In addition to platelet-specific antigens, platelets also express other non-HPA antigens, including blood group ABH antigens and HLA class I antigens. The ABO antibodies are formed naturally, and they are pre-existing in the serum of incompatible naive individuals. In contrast, anti-HLA class I antibodies are developed during incompatible transfusion or pregnancy, and about one-third of multiparous women are sensitized to HLA class I antigens.³³ To prevent potential interference of anti-A, anti-B, and anti-HLA class I antibodies for anti-HPA alloantibody detection in patient sera, we aimed to generate a blood type O HLA class I-negative iPSC founder line. We recently reported the use of integration-free episomal vectors to generate a blood type O iPSC line (OT1-1) derived from human peripheral blood mononuclear cells obtained from a healthy donor.³⁰ β2M, encoded by the B2M gene, is the light chain of HLA class I molecules and is required for trafficking of all class I heavy chains to the cell surface. Disrupting β2M expression using short hairpin RNA or transcription activator-like effector nucleases (TALEN) technology has been carried out for generating HLA class I knockdown/knockout iPSCs.^{34,35} Similarly, we designed a gRNA sequence to target exon 1 of the B2M gene that would introduce an insertion or deletion (indel) into the genome to eliminate expression of β2M, and consequently, the class I HLA heavy chain (FIGS. 7A-7B). After transfection of these CRISPR/Cas9 constructs into the OT1-1 iPSCs, a puromycin-resistant β2M knockout clone (B2MKO) was selected. Flow cytometry analysis confirmed that surface expression of both β2M and the HLA class I heavy chain were abolished in the B2MKO iPSC line (FIG. 7B). These blood group O HLA-negative cells were then used as the founder line into which all additional amino acid substitutions were introduced.

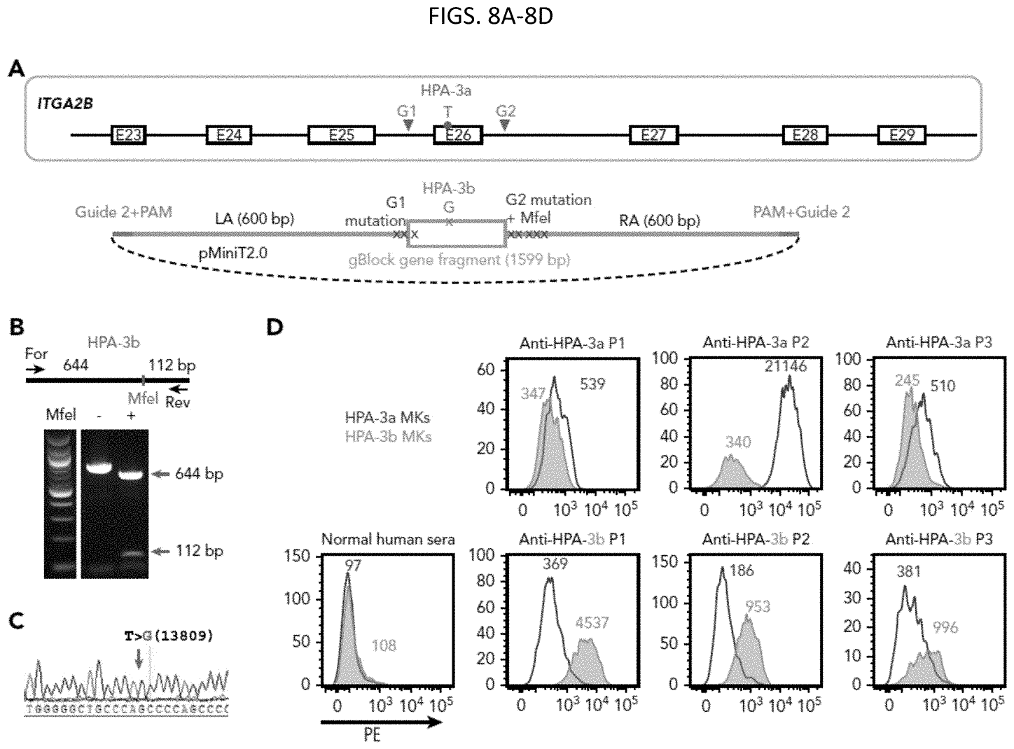

The HPA-3a and HPA-3b polymorphisms are caused by a single T13809G nucleotide substitution in the ITGA2B gene.¹⁵ DNA sequencing of the ITGA2B gene of OT1-1 cells (not shown) showed them to be homozygous for the HPA-3a and HPA-9a alleles. To convert HPA-3a to HPA-3b, a pair of gRNAs flanking exon 26 of ITGA2B were designed to remove the entire exon encoding the HPA-3a epitope (FIG. 8A). The homology-directed repair (HDR) template contained the targeted T13809G point mutation for generating the HPA-3b epitope as well as silent mutations to introduce an MfeI site for genotyping. In addition, guide 2 sequences were added to both ends of the homology arms to linearize the HDR donor inside the cells upon Cas9 cleavage (FIG. 8A), a feature that has previously been found to enhance HDR efficiency.³⁶ After co-transfecting B2MKO (HPA-3a) cells with the CRISPR/Cas9 guide constructs that coexpress a puromycin resistance gene, together with HDR donor plasmids, clones from puromycin-resistant colonies were manually picked, expanded, and subjected to diagnostic MfeI restriction enzyme digestion to identify clones in which biallelic conversion of HPA-3a to HPA-3b had taken place. FIG. 8B shows the MfeI digestion pattern of one such homozygous HPA-3b clone, the T>G 13809 genotype of which was verified by DNA sequencing (FIG. 8C).

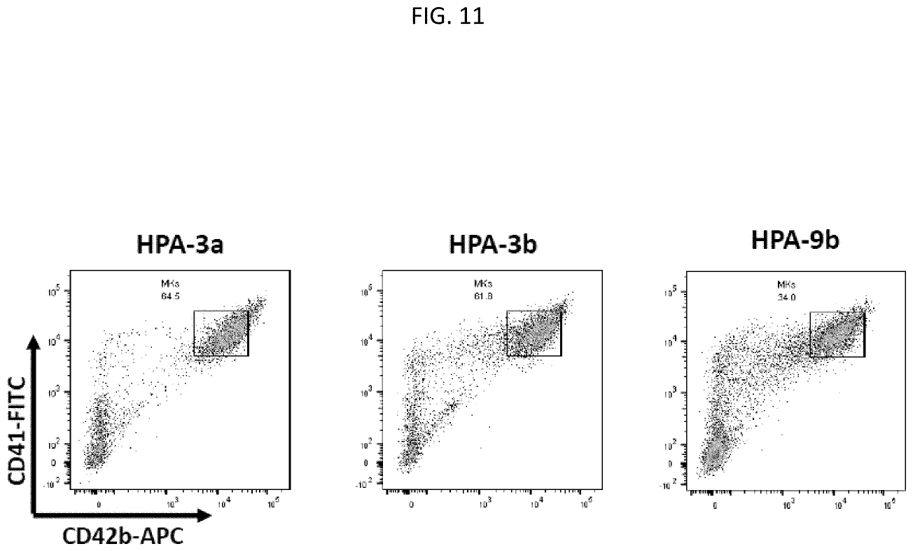

Both HPA-3a and HPA-3b iPSC lines were then differentiated into CD41+/CD42b+ MKs using a previously described serum-free, feeder-free, adherent differentiation system.^{31,32} The MKs derived from the 2 cell lines expressed similar levels of GPIIb (FIG. 11) and showed similar levels of background binding to normal human sera in flow cytometry (FIG. 8D). HPA-3a and HPA-3b patient samples, with the exception of anti-HPA-3a (P3 in FIG. 8D), had tested positive in either the MACE or the MAIPA assay. All 3 HPA-3a patient serum samples reacted with MKs derived from an HPA-3a iPSC line, as expected, and reactivity was lost in MKs in which the HPA-3a allele had been converted to HPA-3b (FIG. 8D). In contrast, HPA-3b-expressing MKs reacted strongly with HPA-3b- but not HPA-3a-specific patient sera. Notably, the anti-HPA-3a sample from P1 was tested because it had a known weak antibody in the MACE assay. As shown in FIG. 8D, it also had the weakest binding of samples tested in our whole-cell flow cytometric detection system, demonstrating comparable sensitivity of this assay to MACE. We also examined the ability of the HPA-3a-expressing MKs to react positively with serum from a patient (P3) containing anti-HPA-3a, but that had been negative in MACE. Similar to previous reports describing certain HPA-3a-specific antibodies that are able to recognize their epitopes only in the context of an intact glycoprotein presented on the surface of intact cells,^{11,12,17,20} sample P3 was positive, although weakly so, against iPSC-derived HPA-3a-but not HPA-3b-expressing MKs. The absence of anti-HLA class I antibodies is a prerequisite for identifying HPA-specific alloantibodies that recognize labile epitopes and that have, to date, been detectable only by using whole-platelet assays. Accordingly, our HLA class I-negative whole-cell assay system seems to provide an important advantage for detecting these types of anti-HPA-3 alloantibodies that are missed or masked when HLA class I antibodies are also present in the sera.

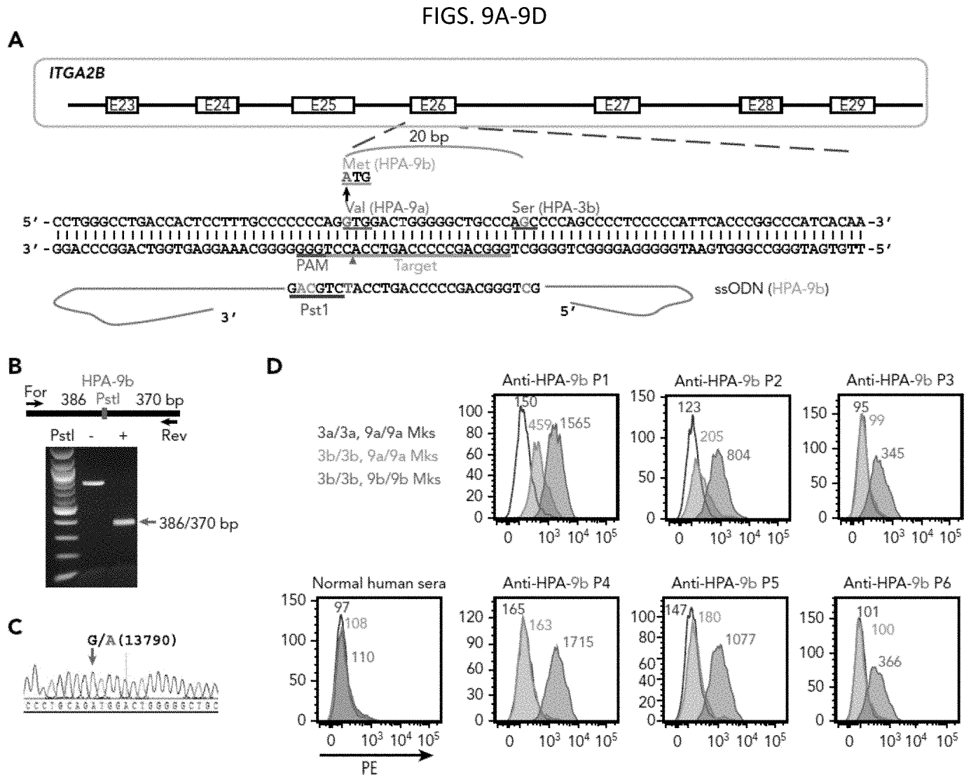

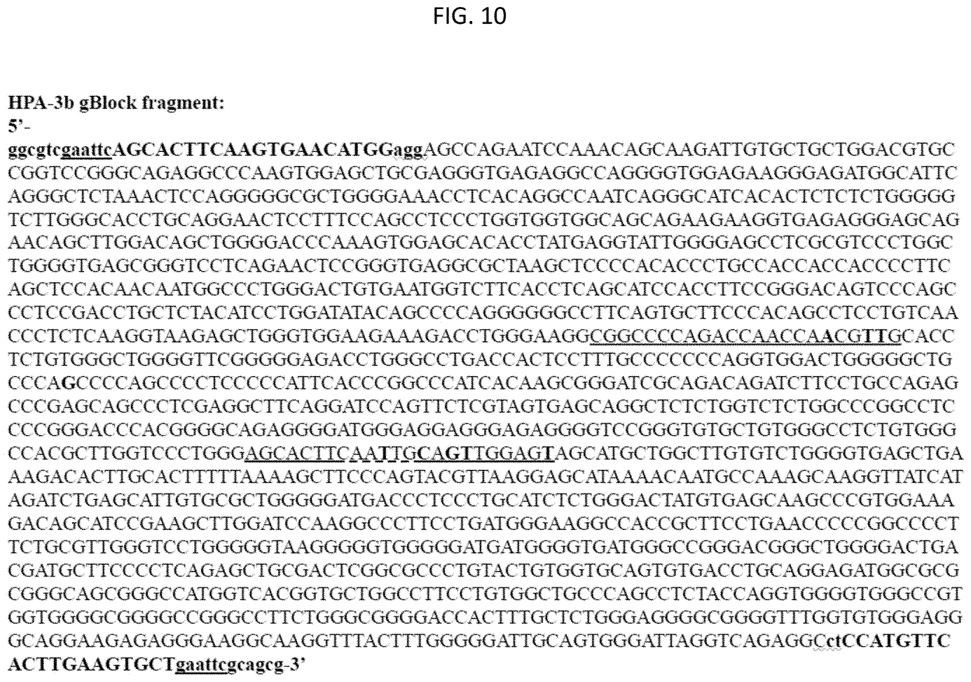

The HPA-9a and HPA-9b polymorphisms are caused by a single G13790A nucleotide substitution in the ITGA2B gene. HPA-9b is genetically linked to HPA-3b because the mutation from HPA-9a to HPA-9b took place on an HPA-3b allele.¹⁶ Thus, we edited HPA-9a to HPA-9b in our newly generated homozygous HPA-3b iPSC line using a guide sequence that targets exon 26 of the ITGA2B gene (FIG. 9A). The ssODN HDR donor contains the targeted G13790A mutation as well as silent mutations to introduce a PstI site for later genotyping. After co-transfection of the CRISPR/Cas9 guide construct and the HDR donor into HPA-3b iPSCs, puromycin-resistant clones were subjected to diagnostic PstI restriction enzyme digestion to identify homozygous HPA-9b clones (FIG. 9B). DNA sequencing confirmed the G>A 13790 mutation (FIG. 9C).

The newly generated HPA-3b/HPA-9b iPSC-derived cell line, when differentiated into CD41+/CD42b+ MKs, expressed levels of GPIIb similar to those of MKs derived from the HPA-3a and HPA-3b iPSC lines described above (FIG. 11) and exhibited very low background binding to normal human sera in flow cytometry (FIG. 9D). Three well-defined HPA-9b-specific alloantibodies (P1-P3) all reacted with HPA-3b/HPA-9b MKs in the flow cytometric test (FIG. 9D). Two of them (P1 and P2) also bound weakly to HPA-3b/HPA-9a MKs (FIG. 9D), but their reactivity was three- to four-fold stronger when the 9b polymorphism was present. Patient (P4-P6) sera were from unresolved cases of FNAIT suspected of containing anti-HPA-9b antibodies as a result of genetic incompatibility for HPA-9b in the parents, but in all 3 cases, the presence of anti-HPA-9b antibody or any other platelet-specific alloantibody could not be detected using standard techniques. As shown in FIG. 9D, anti-HPA-9b alloantibodies in these maternal sera were easily detected with a simple 1-step flow cytometric test using intact class I HLA-negative blood group O iPSC-derived MKs as target cells.

Although it became possible to genotype platelet-specific alloantigens beginning in the early 1990s, serological detection of HPA-specific alloantibodies remains critical for diagnosis, treatment, and prevention of platelet alloimmune disorders, including FNAIT, posttransfusion purpura, and platelet transfusion refractoriness. A wide range of techniques have been developed for HPA alloantibody detection over the last 40 years, but detection remains challenging in many cases. Maternal HPA alloantibodies can be identified in only 20% to 35% of apparent FNAIT cases referred for laboratory investigation.³⁷ In particular, antibodies specific for HPA-3a, HPA-3b, and HPA-9b can be extremely difficult to detect using standard serologic tests.^{38,39}

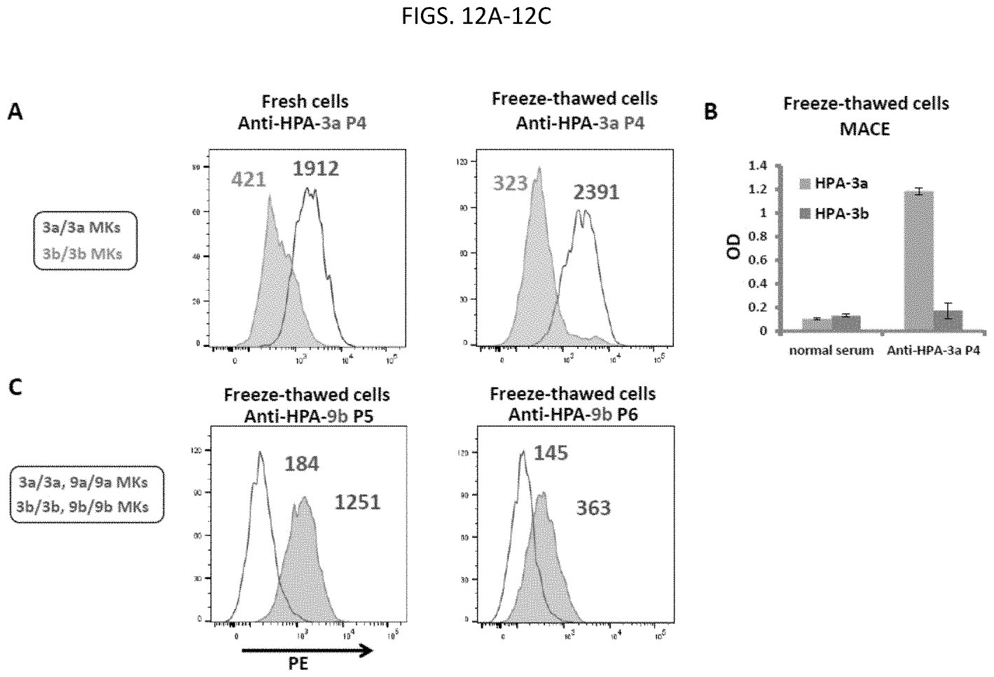

In this embodiment, we demonstrated generation of blood group O HLA class I-negative, HPA-3a, HPA-3b, and HPA-9b allele-specific human iPSC lines using CRISPR gene editing technology. Upon differentiation, the iPSC-derived MKs expressed allele-specific HPAs on their surface that were easily adapted for flow cytometric detection of anti-HPA-3a, HPA-3b, and HPA-9b alloantibodies in patient sera. Importantly, patient sera that had previously been identified using standard clinical enzyme-linked immunosorbent assay-based MAIPA and MACE assays, as well as samples that had been negative using these methods could be typed using these intact, allele-specific bioengineered MKs. In contrast to MACE or MAIPA assays, which are time-consuming, labor-intensive, and difficult to standardize, we suggest that the flow cytometric assay described herein will be fast, simple to perform with either fresh or previously frozen cells (FIGS. 12A-12C), and require only 50 to 100 mL of patient serum.

Characterizing the precise specificity of anti-HPA alloantibodies present in maternal sera has historically required access to a well-characterized panel of platelets that have a wide variety of phenotypic specificities. This can be challenging, especially when platelets expressing rare low-frequency HPAs are needed. Previous attempts to circumvent this problem using transfected cell lines expressing allele-specific human platelet membrane glycoproteins have encountered technical issues that have precluded their wide-spread adoption. For example, COS cells (derived from fibroblasts of the African green monkey) expressing allelic isoforms of human GPIIb/IIIa are unable to detect a significant number of HPA-3a and HPA-3b alloantisera,⁴⁰ likely because of their requirement for species-specific glycans that form part of their recognition epitope.²¹ Chinese hamster ovary (CHO) cells seem to be particularly poor in their ability to display human platelet alloantigenic epitopes, because a substantial number of human anti-HPA-3a,²¹ anti-HPA-9b,²⁵ and anti-Lapa4 human alloantisera fail to react with human GPIIb expressed in this cell line. Transformed human cancer cell lines are not immune to this problem either. Hayashi et al⁴¹ discovered that only 4 of 6 anti-HPA-3a alloantisera are reactive with GPIIb derived from human erythroleukemia K562 cells, suggesting a limitation of using these cells in diagnostic HPA testing.

The inability to consistently detect anti-HPA-3a alloantibodies using cells of non-platelet origin can be at least partially attributed to the heterogeneous requirement^{21-23,40} for O-linked oligosaccharide chains that emanate from serine residues 845 and 847,⁴² both of which are proximal to the Ile843Ser polymorphic residue that defines the HPA-3 alloantigen system.^{15,40} In particular, although 0-glycans attached to GPIIb Ser847 have been shown to participate in the formation of the HPA-3a epitope,²⁴ the involvement of glycans in the formation of the HPA-3b epitope remains unclear, especially because it is formed as a result of replacing Ile843 (HPA-3a) with still another serine residue that has the potential to host a third glycan in the immediate vicinity of the polymorphic amino acid. The effect, if any, of these glycans on the HPA-9b alloantigenic epitope is completely unknown.

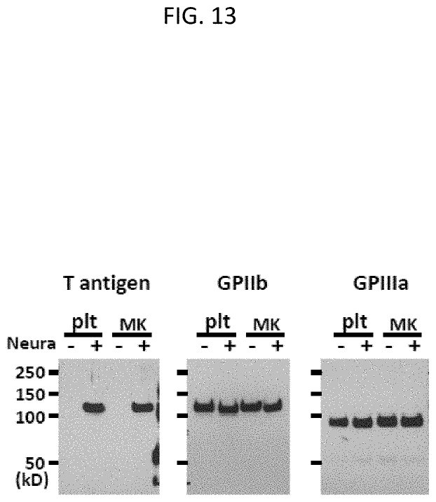

Mucin-type O-glycosylation is controlled by a large family of >20 genes that encode UDP-GalNAc:polypeptide GalNAc transferases (GalNAc-Ts) that are differentially expressed in cells and tissues, and marked changes in expression are also found in cancer cells.⁴³ Because of the differential regulation of mucin-type O-glycosylation in cells and tissues, human GPIIb synthesized by non-human cells (COS, CHO) or human cancer cell lines (K562) are likely to express completely different glycan chains than would GPIIb produced in human platelets or MKs. Preliminary studies performed in our laboratory suggest that GPIIb expressed in iPSC-derived MKs contains the same sialylated T-antigens present in native O-glycosylated GPIIb from human platelets⁴² (FIG. 13), making these cells uniquely suited for detecting HPA alloantibodies that recognize O-glycan-dependent epitopes.

Maternal sera containing platelet-specific alloantibodies often also harbor alloantibodies specific for 1 or more HLA class I polymorphisms. Thus, a major advantage of using HPA allele-specific MKs for detection of platelet-specific alloantibodies is that they can be engineered to not express the heavy and light chains of the HLA class I protein heterodimer complex, thereby lending themselves to widely available detection methods, like flow cytometry, that use intact cells. By knocking out β2M in our iPSC founder line (FIGS. 7A-7B), the resulting iPSCs could be easily modified using CRISPR/Cas9 gene editing technology to generate a series of cell lines that, upon differentiation, express homozygous allelic isoforms of GPIIb carrying the HPA-3a, HPA-3b, or HPA-9b alloantigenic determinants on their cell surface (FIGS. 8A-8D and 9A-9D). Because they are all derived from a single clonal iPSC line, these cells should provide extremely high specificity for HPA detection because they all share an identical genetic background, and therefore phenotype, with the sole exception of the targeted polymorphic HPA allele. Because these HPA allele-specific MKs express GPIIb allele in homozygous form, the increase in alloantigen density on the cell surface should also result in increased sensitivity. This was demonstrated by the ability to identify and type anti-HPA alloantibody reactivity in several maternal sera that had previously shown weak or negative reactivity using currently existing methodology (FIGS. 9A-9D).

Recent advances in techniques, including the establishment of human iPSCs and CRISPR/Cas9-mediated gene editing have opened up new possibilities for stem cell-based therapies in a wide variety of disorders, especially for transfusion medicine. Solid groundwork has been laid for the ex vivo production of MKs and platelets, and the field is advancing rapidly. Platelet products are qualitatively and quantitatively approaching a clinically applicable level owing to advances in expandable MK lines, platelet-producing bioreactors, and novel reagents.^{28,29} Undoubtedly, the capacity to generate large-scale donor-independent MKs and platelets will promote their clinical translation and application sooner than had been anticipated. Incorporating our system into expandable MK cell lines holds great potential for production of designer platelets for diagnostic, investigative, and ultimately therapeutic use. We envision a future in which specialty units of in vitro-generated HPA-matched iPSC-derived platelets are used clinically to complement donor-derived platelets.

### REFERENCES

1. Curtis B R. Recent progress in understanding the pathogenesis of fetal and neonatal alloimmune thrombocy-topenia. Br J Haematol. 2015; 171(5): 671-682.
2. Sullivan M J, Kuhlmann R, Peterson J A, Curtis B R. Severe neonatal alloimmune thrombocytopenia caused by maternal sensitization against a new low-frequency alloantigen (Domb) located on platelet glycoprotein Ma. Transfusion. 2017; 57(7): 1847-1848.
3. Poles A, Lucas G, Green F, et al. Neonatal alloimmune thrombocytopenia due to a new alloantigen Bl(a) defined by an Asp458Gly substitution in GPIIIa. Transfusion. 2019; 59(1): 396-404.
4. Wihadmadyatami H, Heidinger K, Ro\ddot{ }der L, et al. Alloantibody against new platelet alloantigen (Lap(a)) on glycoprotein Hb is responsible for a case of fetal and neonatal alloimmune thrombocytopenia. Transfusion. 2015; 55(12):2920-2929.
5. Santoso S, Kiefel V, Richter I G, et al. A functional platelet fibrinogen receptor with a deletion in the cysteine-rich repeat region of the beta(3) integrin: the Oe(a) alloantigen in neonatal alloimmune thrombocytopenia. Blood. 2002; 99(4): 1205-1214.
6. Newman P J, McFarland J G, Aster R H. The alloimmune thrombocytopenias. In: Loscalzo J, Schafer A I, eds. Thrombosis and Hemorrhage, Philadelphia, Pa.: Lippincott Williams and Wilkins; 2003:441-456.
7. Hod E, Schwartz J. Platelet transfusion refractoriness. Br J Haematol. 2008; 142(3): 348-360.
8. Hayashi T, Hirayama F. Advances in alloimmune thrombocytopenia: perspectives on current concepts of human platelet antigens, antibody detection strategies, and genotyping. Blood Transfus. 2015; 13(3):380-390.
9. Menitove J E, Pereira J, Hoffman R, Anderson T, Fried W, Aster R H. Cyclic thrombocytopenia of apparent autoimmune etiology. Blood. 1989; 73(6):1561-1569.
10. Kiefel V, Santoso S, Weisheit M, Mueller-Eckhardt C. Monoclonal antibody-specific immobilization of platelet antigens (MAIPA): a new tool for the identification of platelet-reactive antibodies. Blood. 1987; 70(6):1722-1726.
11. Harrison C R, Curtis B R, McFarland J G, Huff R W, Aster R H. Severe neonatal alloimmune thrombocytopenia caused by antibodies to human platelet antigen 3a (Baka) detectable only in whole platelet assays. Transfusion. 2003; 43(10):1398-1402.
12. Barba P, Pallare's P, Nogue's N, et al. Post-transfusion purpura caused by anti-HPA-3a antibodies that are only detectable using whole platelets in the platelet immunofluorescence test. Transfus Med. 2010; 20(3): 200-202.
13. von dem Borne A E, von Riesz E, Verheugt F W, et al. Baka, a new platelet-specific antigen involved in neonatal allo-immune thrombocytopenia. Vox Sang. 1980; 39(2):113-120.
14. van der Schoot C E, Wester M, Von Dem Borne A E, Huisman H G. Characterization of platelet-specific alloantigens by immunoblotting: localization of Zw and Bak antigens. Br J Haematol. 1986; 64(4):715-723.
15. Lyman S, Aster R H, Visentin G P, Newman P J. Polymorphism of human platelet membrane glycoprotein IIb associated with the Baka/Bakb alloantigen system. Blood. 1990; 75(12):2343-2348.
16. Noris P, Simsek S, de Bruijne-Admiraal L G, et al. Max(a), a new low-frequency platelet-specific antigen localized on glycoprotein IIb, is associated with neonatal alloimmune thrombocytopenia. Blood. 1995; 86(3):1019-1026.
17. Lin M, Shieh S H, Liang D C, Yang T F, Shibata Y. Neonatal alloimmune thrombocytopenia in Taiwan due to an antibody against a labile component of HPA-3a (Baka). Vox Sang. 1995; 69(4):336-340.
18. Boehlen F, Kaplan C, de Moerloose P. Severe neonatal alloimmune thrombocytopenia due to anti-HPA-3a. Vox Sang. 1998; 74(3): 201-204.
19. Glade-Bender J, McFarland J G, Kaplan C, Porcelijn L, Bussel J B. Anti-HPA-3A induces severe neonatal alloimmune thrombocytopenia. J Pediatr. 2001; 138(6):862-867.
20. Kataoka S, Kobayashi H, Chiba K, et al. Neonatal alloimmune thrombocytopenia due to an antibody against a labile component of human platelet antigen-3b (Bak b). Transfus Med. 2004; 14(6):419-423.
21. Socher I, Zwingel C, Santoso S, Kroll H. Heterogeneity of HPA-3 alloantibodies: consequences for the diagnosis of alloimmune thrombocytopenic syndromes. Transfusion. 2008; 48(3):463-472.
22. Take H, Tomiyama Y, Shibata Y, et al. Demonstration of the heterogeneity of epi-topes of the platelet-specific alloantigen, Baka. Br J Haematol. 1990; 76(3):395-400.
23. Djaffar I, Vilette D, Pidard D, Wautier J L, Rosa J P. Human platelet antigen 3 (HPA-3): localization of the determinant of the alloantibody Lek(a) (HPA-3a) to the C-terminus of platelet glycoprotein IIb heavy chain and contribution of O-linked carbohydrates. Thromb Haemost. 1993; 69(5):485-489.
24. Calvete J J, Muñiz-Diaz E. Localization of an O-glycosylation site in the alpha-subunit of the human platelet integrin GPIIb/IIIa involved in Baka (HPA-3a) alloantigen expression. FEBS Lett. 1993; 328(1-2):30-34.
25. Peterson J A, Balthazor S M, Curtis B R, McFarland J G, Aster R H. Maternal alloimmunization against the rare platelet-specific antigen HPA-9b (Max a) is an important cause of neonatal alloimmune thrombocytopenia. Transfusion. 2005; 45(9):1487-1495.
26. Kaplan C, Porcelijn L, Vanlieferinghen P, et al. Anti-HPA-9bw (Maxa) fetomaternal alloimmunization, a clinically severe neonatal thrombocytopenia: difficulties in diagnosis and therapy and report on eight families. Transfusion. 2005; 45(11):1799-1803.
27. Peterson J A, Gitter M, Bougie D W, et al. Low-frequency human platelet antigens as triggers for neonatal alloimmune thrombocytopenia. Transfusion. 2014; 54(5):1286-1293.
28. Moreau T, Evans A L, Vasquez L, et al. Large-scale production of megakaryocytes from human pluripotent stem cells by chemically defined forward programming Nat Commun. 2016; 7(1):11208.
29. Ito Y, Nakamura S, Sugimoto N, et al. Turbulence activates platelet biogenesis to enable clinical scale ex vivo production. Cell. 2018; 174(3):636-648.e18.
30. Zhang N, Newman P J. Packaging functionally important plasma proteins into the a-granules of human-induced pluripotent stem cell-derived megakaryocytes. J Tissue Eng Regen Med. 2019; 13(2):244-252.
31. Mills J A, Paluru P, Weiss M J, Gadue P, French D L. Hematopoietic differentiation of pluripotent stem cells in culture. In: Walker J M, ed. Methods in Molecular Biology, New York, N.Y.: Springer SciencelBusiness Media; 2014: 181-194.
32. Paluru P, Hudock K M, Cheng X, et al. The negative impact of Wnt signaling on mega-karyocyte and primitive erythroid progenitors derived from human embryonic stem cells. Stem Cell Res (Amst). 2014; 12(2):441-451.
33. King K E, Kao K J, Bray P F, et al. The role of HLA antibodies in neonatal thrombocytopenia: a prospective study. Tissue Antigens. 1996; 47(3):206-211.
34. Feng Q, Shabrani N, Thon J N, et al. Scalable generation of universal platelets from human induced pluripotent stem cells. Stem Cell Reports. 2014; 3(5):817-831.
35. Borger A K, Eicke D, Wolf C, et al. Generation of HLA-universal iPSC-derived megakaryo-cytes and platelets for survival under refractoriness conditions. Mol Med. 2016; 22: 274-285.
36. Zhang J P, Li X L, Li G H, et al. Efficient precise knockin with a double cut HDR donor after CRISPR/Cas9-mediated double-stranded DNA cleavage. Genome Biol. 2017; 18(1):35.
37. Peterson J A, McFarland J G, Curtis B R, Aster R H. Neonatal alloimmune thrombocytopenia: pathogenesis, diagnosis and management. Br J Haematol. 2013; 161(1):3-14.
38. Wu G G, Kaplan C, Curtis B R, Pearson H A. Report on the 14th International Society of Blood Transfusion Platelet Immunology Workshop. Vox Sang. 2010; 99(4):375-381.
39. Sachs U J, Kiefel V, Kroll H, Bein G, Santoso S. Report on the 15th International Society of Blood Transfusion Platelet Immunology Workshop. Vox Sang. 2012; 103(4):343-351.
40. Goldberger A, Kolodziej M, Poncz M, Bennett J S, Newman P J. Effect of single amino acid substitutions on the formation of the N A and Bak alloantigenic epitopes. Blood. 1991; 78(3): 681-687.
41. Hayashi T, Amakishi E, Matsuyama N, et al. Establishment of a cell line panel as an alternative source of platelet antigens for a screening assay of anti-human platelet antibodies. Transfus Med. 2011; 21(3):199-204.
42. King S L, Joshi H J, Schjoldager K T, et al. Characterizing the O-glycosylation landscape of human plasma, platelets, and endothelial cells. Blood Adv. 2017; 1(7):429-442.
43. Bennett E P, Mandel U, Clausen H, Gerken T A, Fritz T A, Tabak L A. Control of mucin-type O-glycosylation: a classification of the polypeptide GalNAc-transferase gene family. Glycobiology. 2012; 22(6):736-756.

### Example 3

Chronically transfused recipients with sickle cell disease, thalassemia, and warm autoimmune hemolytic anemia are constantly exposed to the dangers of red blood cell (RBC) alloimmunization. Correct identification of alloantibodies is vital to find a compatible donor unit for such patients. Diagnostic labs performing high-complexity testing are dependent on difficult-to-procure rare donor RBCs as a reagent in antibody identification panels.

Approx. 2-6% of patients receiving red cell transfusions develop antibodies to incompatible antigens present on donor red cells. If you have an antibody and receive incompatible red cells, you can develop life-threatening hemolysis. Therefore, every person requiring a blood transfusion has to be tested for the presence of blood group antibodies before each and every transfusion. This adds up to ˜5 million patients tested/transfused annually, and probably more than 10 million red cell typing tests performed each year. The RBCs currently used for making typing panels are obtained exclusively from blood donors, placed into commercially prepared kits, and used by IRLs all across the country.

Additionally, several patient populations exist that need specialty red cell units for transfusions. For example, patients with sickle cell disease, Thalassemia, myelodysplasia, certain cancers, or patients with rare blood groups like Bombay blood group ( 1/250,000), Rh null phenotype ( 1/6000,000). People with sickle cell disease and thalassemia, and random transfusion recipients have rare blood types and often have a hard time getting patient-matched red cells. For example, we see rare types from the Rh, MNS, and Kell/Kx to name 3 of the 36 blood group systems. Patients receiving imperfectly-matched units can develop red cell antibodies that react against non-self red blood cell antigens. These antibodies bind to red cells and cause their lysis, releasing toxic free hemoglobin into the blood stream—a condition known as a transfusion-associated hemolytic reaction.

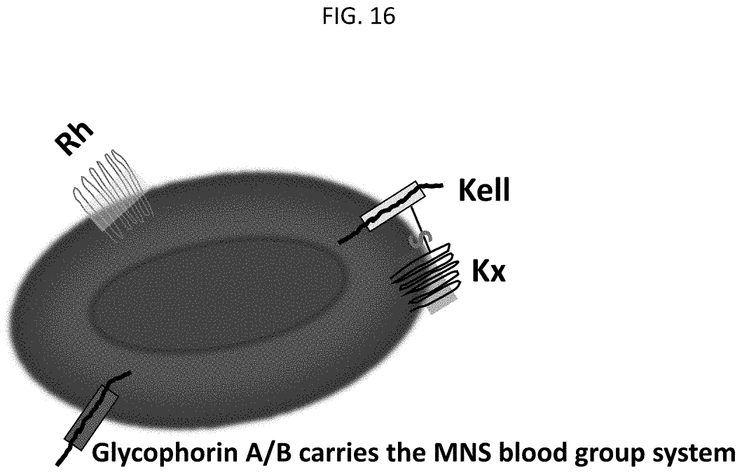

Examples of red cell antigens are provided in FIG. 14 and described herein. Additional antigen systems are shown in FIG. 16.

To knock out surface expression of the protein carrying the Kell polymorphism, a missense mutation within the gene for Kx—an accessory protein required for surface expression of Kell—was introduced using CRISPR/Cas9 gene editing. The Kx protein itself is also missing in a rare condition known as McLeod syndrome. To knock out the Rh blood group system proteins RhD and RhCE, a missense mutation within the gene for RhAG—an accessory protein required for surface expression of RhD and RhCE—was introduced using CRISPR/Cas9 gene editing. Guide RNAs used to knock out RhAg, Kx, and Glycophorins A and B are shown below.

Guide RNA sequences to knock out the MNS blood group system—bold and italicized nucleotides are added to facilitate cloning into a Bbsl digester Cas9 expressing plasmid.

| | Exon | | SEQ | |
|---|---|---|---|---|
| Name | targeted | Sequence (5′-3′) | ID NO: | PAM |
| G1 | Exon 2 | CACCGATCAGCATTAAGTACCACTG | 63 | AGG |
| | | AAACCAGTGGTACTTAATGCTGATC | 64 | |
| G2 | Exon 2 | CACCGAGTGTGCATTGCCACCTCAG | 65 | TGG |
| | | AAACCTGAGGTGGCAATGCACACTC | 66 | |
| G3 | Exon 2 | CACCGAGCATTAAGTACCACTGAGG | 67 | TGG |
| | | AAACCCTCAGTGGTACTTAATGCTC | 68 | |
| G4.1 | Exon 4 | CACCGGTCCATCGTTTCACTGTACC | 69 | AGG |
| | (GYPA) | AAACGGTACAGTGAAATGATGGACC | 70 | |
| G4.2 | Exon 4 | CACCGGCCCATCATTTCTCTGAACC | 71 | AGG |
| | (GYPB) | AAACGGTTCAGAGAAACGATGGGCC | 72 | |

Guide RNA sequences to knock out the Kell blood group system—bold and italicized nucleotides are added to facilitate cloning into a Bbsl digester Cas9 expressing plasmid.

| | Exon | | SEQ | |
|---|---|---|---|---|
| Name | targeted | Sequence (5′-3′) | ID NO: | PAM |
| G1 | Intro 1-2 | CACCGATAAAAGTGCATGTTAGAGG | 73 | AGG |
| | | AAACCCTCTAACATGCACTTTTATC | 74 | |
| G2 | Intron 1-2 | CACCGGAATATTTTTCTTACTCCTA | 75 | GGG |
| | | AAACTAGGAGTAAGAAAAATATTCC | 76 | |
| G3 | Exon 2 | CACCGATGTCAGTATCACCAAGAAG | 77 | AGG |
| | | AAACCTTCTTGGTGATACTGACATC | 78 | |
| G4 | Intron 2-3 | CACCGAACAAGACATGTGAGTGAAG | 79 | GGG |
| | | AAACCTTCACTCACATGTCTTGTTC | 80 | |
| G5 | Intron 2-3 | CACCGTAAGTATTTCAACAATGAGA | 81 | AGG |
| | | AAACTCTCATTGTTGAAATACTTAC | 82 | |

Guide RNA sequences to knock out RhAg—bold and italicized nucleotides are added to facilitate cloning into a Bbsl digester Cas9 expressing plasmid.

| | Exon | | SEQ | |
|---|---|---|---|---|
| Name | targeted | Sequence (5′-3′) | ID NO: | PAM |
| G1 | Exon 2 | CACCGCAGTGGGGCACTATTGTACA | 83 | GGG |
| | | AAACTGTACAATAGTGCCCCACTGC | 84 | |
| G2 | Exon 2 | CACCGCCTGTACAATAGTGCCCCAC | 85 | TGG |
| | | AAACGTGGGGCACTATTGTACAGGC | 86 | |
| G3 | Exon 2 | CACCGCAACCTACTCGTTGCTGCTT | 87 | TGG |
| | | AAACAAGCAGCAACGAGTAGGTTGC | 88 | |
| G4 | Exon 3 | CACCGATATCTTTTGGAGCTGTCC | 89 | TGG |
| | | AAACGGACAGCTCCAAAAGATATC | 90 | |
| GS | Exon 3 | CACCGCACTAACCAGGTATTCATTG | 91 | TGG |
| | | AAACCAATGAATACCTGGTTAGTGC | 92 | |
| G6 | Exon 3 | CACCGGGTGGGGCTCGTTTTTCCC | 93 | AGG |
| | | AAACGGGAAAAACGAGCCCCACCC | 94 | |

The RHAG gene encodes a clinically important protein (RhAG) responsible for bringing the immunogenic Rh blood group system proteins RhD and RhCE to the RBC surface. Though present on a different chromosome, the RhAg gene encodes a 12 membrane-spanning protein that complexes with the gene products of RhD and CE and is required for their surface expression. (FIG. 19) Introducing a missense mutation in any one of the three will prevent expression of all three proteins. Since RhD and CE are both clinically important to test for, deleting RhAg accomplishes both goals with a single mutation. In this study, CRISPR-Cas9 gene editing technology was utilized to knock out the RHAG gene in immortalized human-inducible pluripotent stem cells (iPSCs), followed by differentiation into primitive erythroblasts.

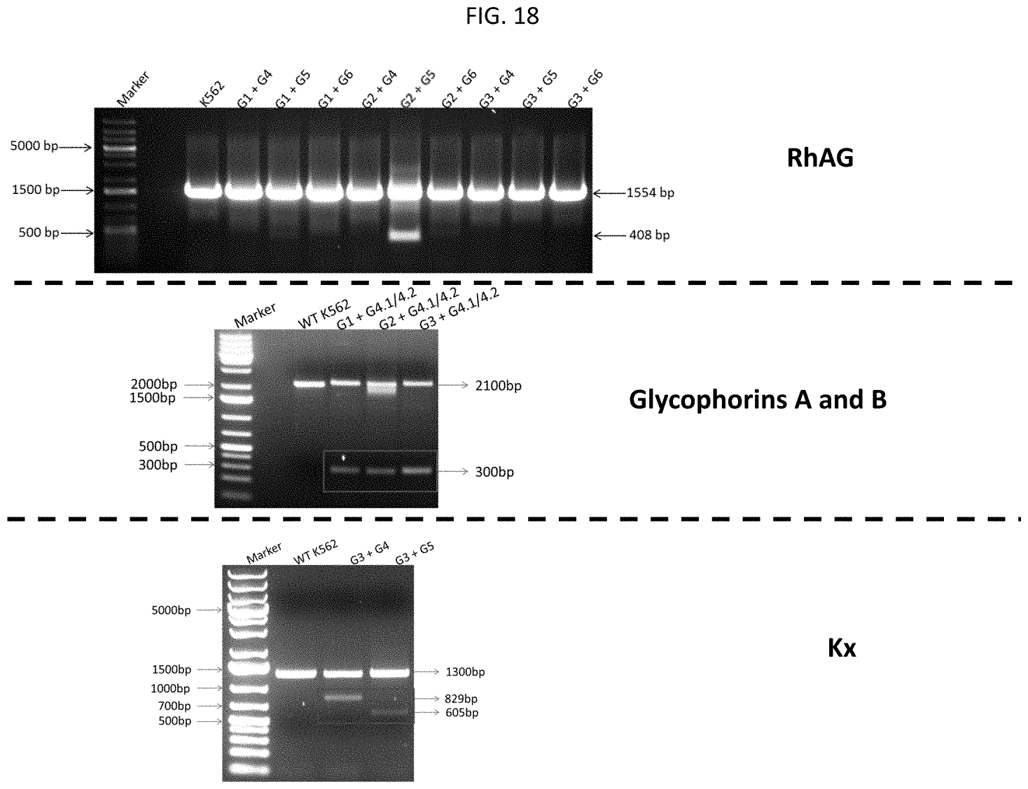

Guide RNAs (gRNA) designed to target a region spanning Exons 2 and 3 of the RhAG gene were cloned into a GFP-Cas9 expression vector and transfected into K562 erythro-leukemia cells to test their ability to mediate gene deletion. (FIG. 18) GFP-positive cells were sorted, grown for a week, genotyped and phenotyped to confirm RHAG/RhAG deletion. gRNAs found to be the most efficient at gene deletion were then cloned into a puromycin-Cas9 expression vector and transfected into a blood group 0, HLA Class 1-negative iPSC founder line. While gRNA sequences were verified in K562 erythro-leukemia cells, this step is not required and gRNAs can be verified directly in iPS cells. Because iPS cell culture maintenance is more costly and time consuming, validation of gRNA sequences can be done in a complementary cell line, for example K562 erythro-leukemia cell or DAMI cells, it is not required for practice of the methods described herein.

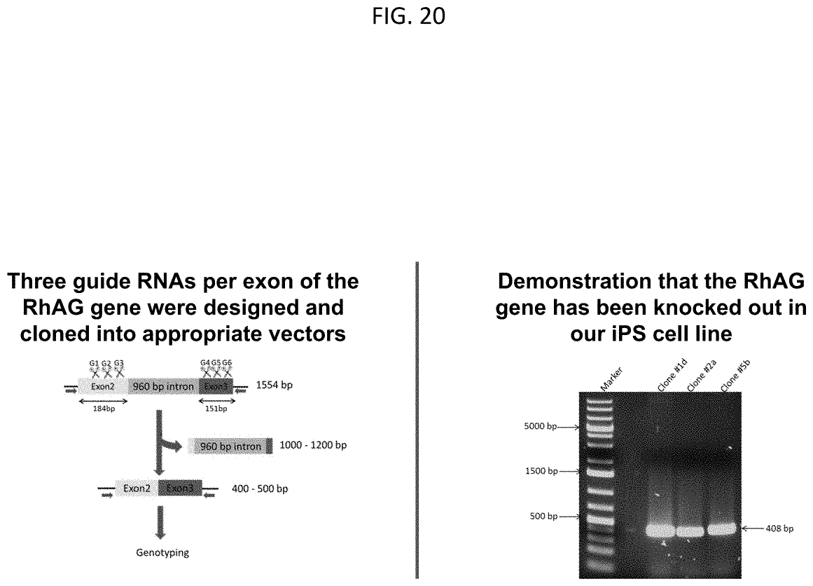

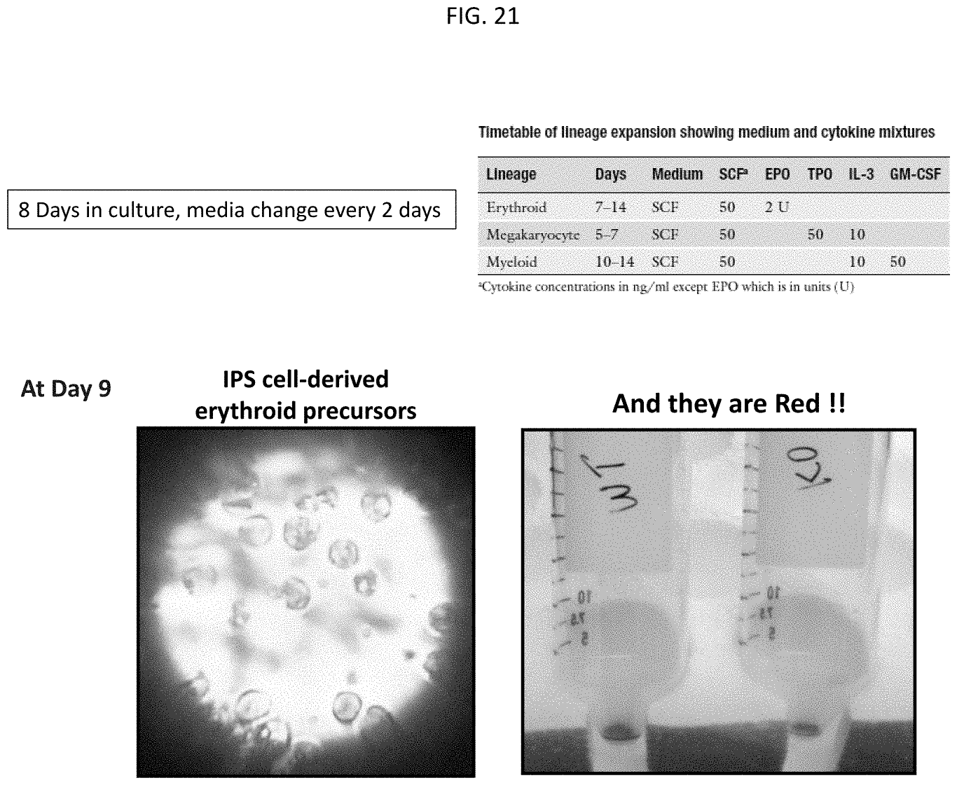

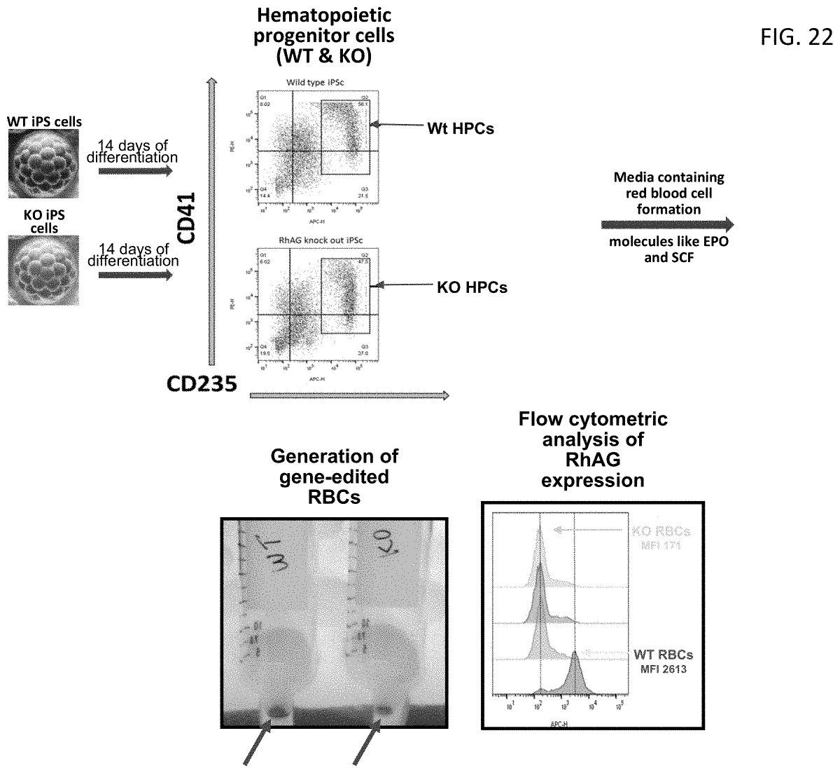

Puromycin-resistant iPSC clones were picked, expanded, and genotyped to confirm the RhAG deletion. (FIG. 20) Wild-type (WT) and knock-out (RhAG KO) iPSC lines were then differentiated into hematopoietic progenitor cells and primitive erythroblasts using a specialized cocktail of RBC-promoting cytokines and growth factors. (FIG. 21) See Mills et al. (“Hematopoietic differentiation of pluripotent stem cells in culture,” Hematopoietic Stem Cell Protocols. Methods in Molecular Biology (Methods and Protocols), vol. 1185, Humana Press, New York, N.Y.).

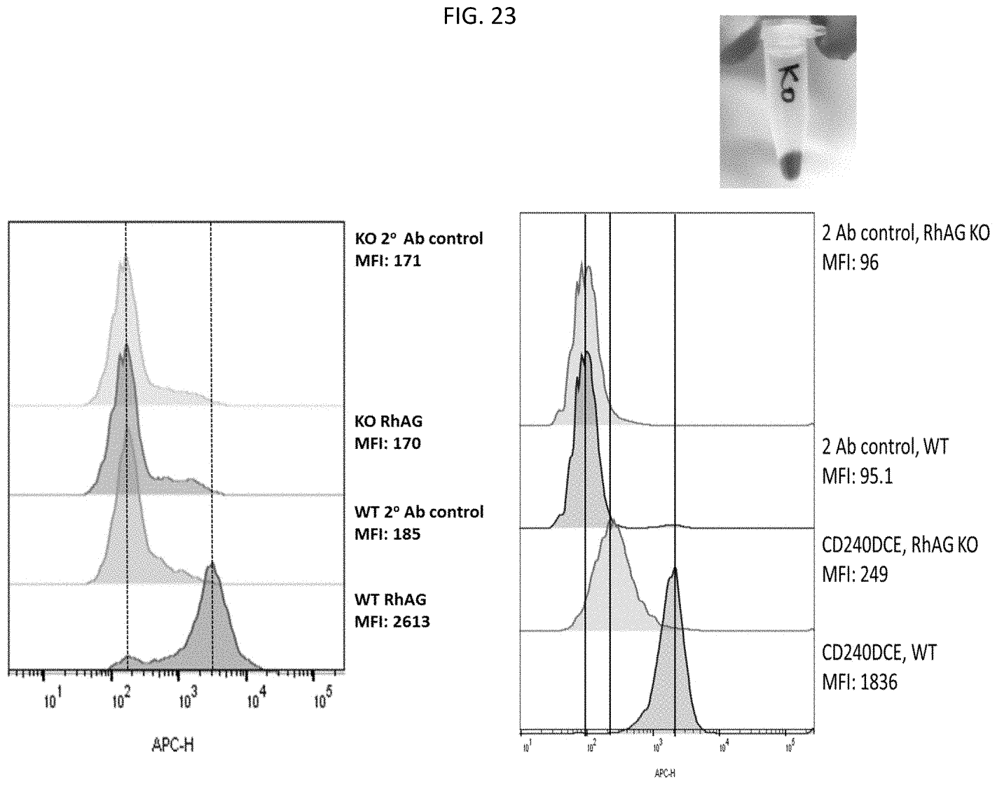

The resulting erythroblasts were evaluated by flow cytometry for a panel of RBC antigens that included RhAG, RhDCE, Kell, Duffy (ACKR1), Glycophorins A/B, transferrin receptor, and Band 3. (FIG. 23). The antibody CD240 DCE detects all three antigens. Note it is positive on wild-type cells (mfi 1836) and negative on RhAgKO cells (mfi 249). Mfi=median fluorescence intensity by flow cytometric analysis.

Results/Findings: Flow cytometry forward- and side-scatter profiles of iPSC-derived primitive erythroblasts were similar to that of mature human RBCs. Both WT and RhAG KO erythroblasts expressed Kell, Glycophorin A/B, transferrin receptor, and Band 3, but not Duffy. The results are in accordance with the genetic make-up of the iPSC line used in the study, which started out Duffy negative. Importantly, whereas WT iPSC-derived erythroblasts strongly expressed RhAG/RhDCE, those in which the RhAG gene had been knocked out were negative for both RhAG as well RhDCE.

Conclusions: Red cell precursors derived from CRISPR/Cas9 gene-edited human iPSCs missing one or more blood group systems may be able to provide rare donor reagent RBCs for the production and use in antibody identification panels, currently available nationwide for the determination of pre-transfusion red cell alloimmunization.

### Example 4

Antibody identification is an important component of compatibility testing in blood transfusions which is a procedure performed to determine if a particular unit of blood can be transfused safely into a certain patient. Subjects requiring regular transfusions include those with sickle cell anemia, Thalassemia, myelodysplastic syndromes, and certain other cancers. These transfusions often carry the risk of erythrocyte alloimmunization, thereby increasing the chances of potential life-threatening delayed hemolytic transfusion reactions. Erythrocyte alloimmunization is the development of antibodies against transfused donor cells. Genetic as well as patient related factors like impaired immunoregulatory abnormalities, also high degree of polymorphisms in the immunogenic RBC antigens are few of the many reasons thought to be a likely driving force for alloantibody production. Therefore, it is imperative to identify such alloantibodies in repeatedly transfused patients and a corresponding antigen negative blood unit could be given. This could minimize the antibody mediated destruction of transfused red cells bringing some respite to the diseased subjects. Diagnostic labs identify such alloantibodies using an antibody identification panels.

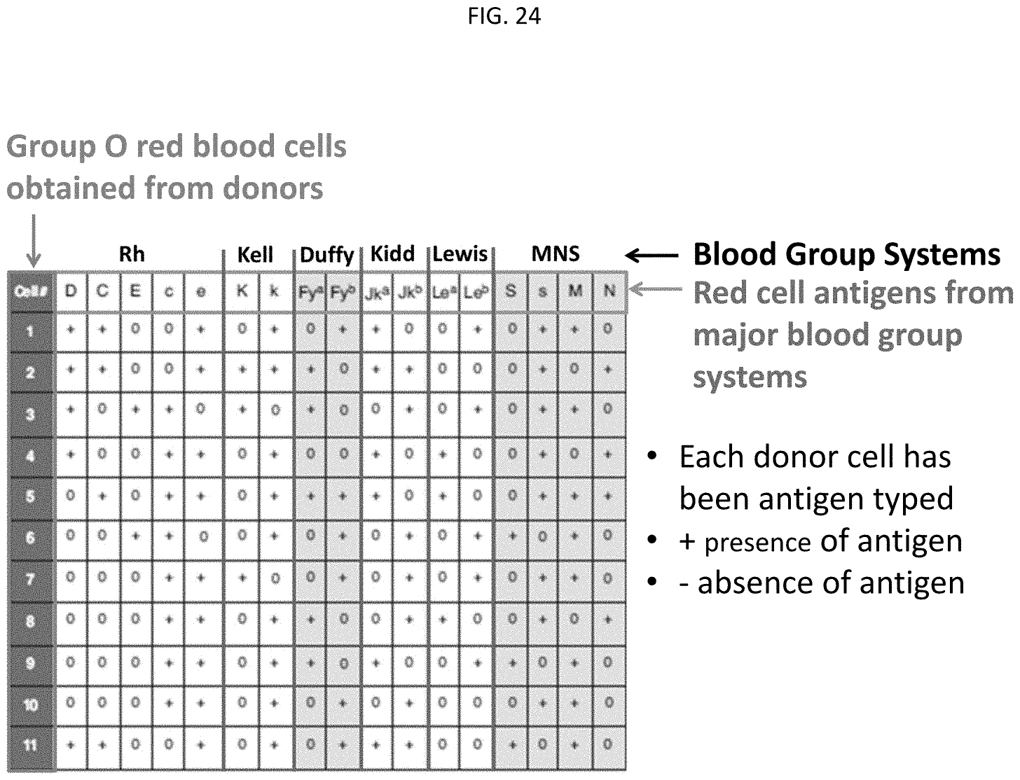

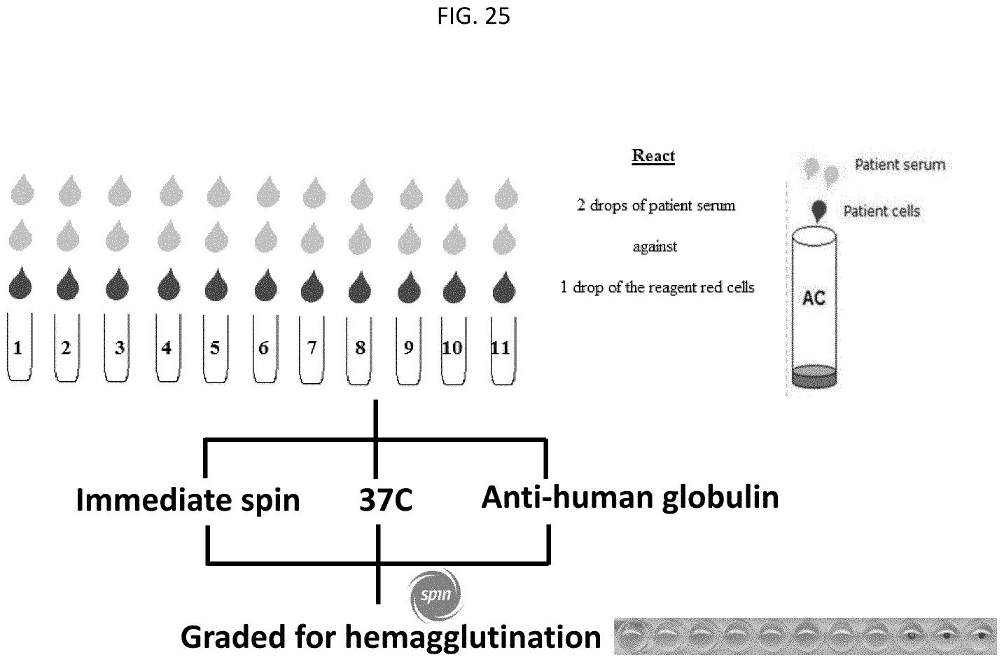

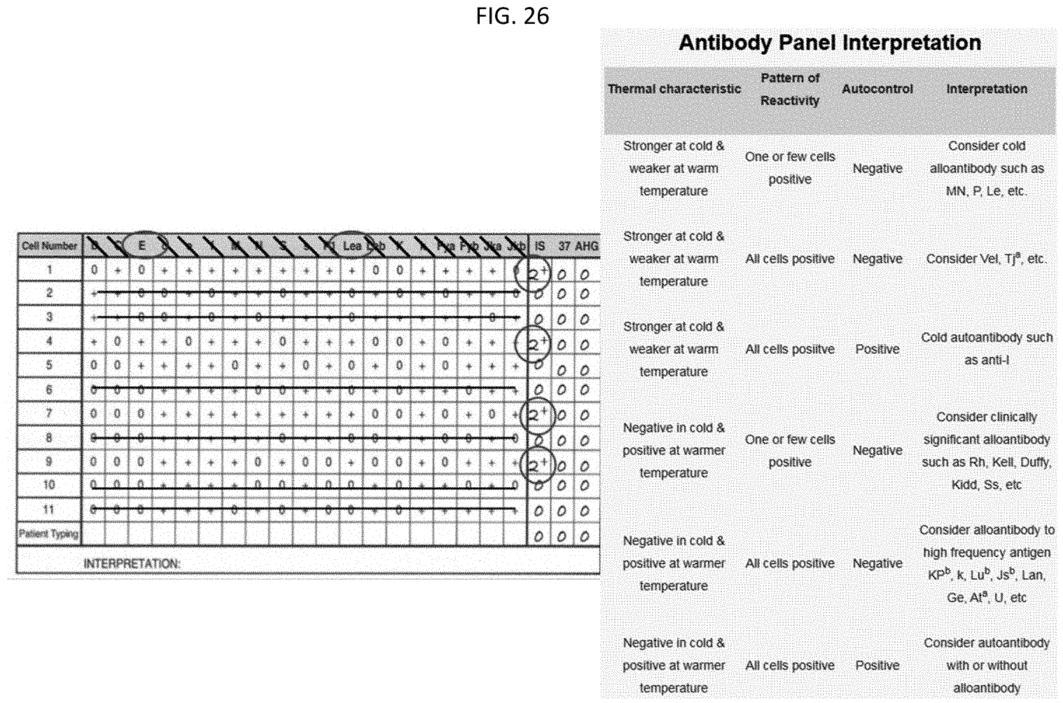

An example of an antibody identification panel is shown in FIG. 24. In general, a panel of reagent red blood cells with known phenotypes are mixed with a patient's serum or plasma in a 1:2 ratio. An auto control tube is also added to the identification process to rule out any auto antibodies. The pattern of positive and negative reactions with these cells identifies the antigen against which the antibody is directed. This method is outlined in FIG. 25. A sample of the data analysis process is shown in FIG. 26, highlighting the range of techniques and temperatures used for antibody identification.

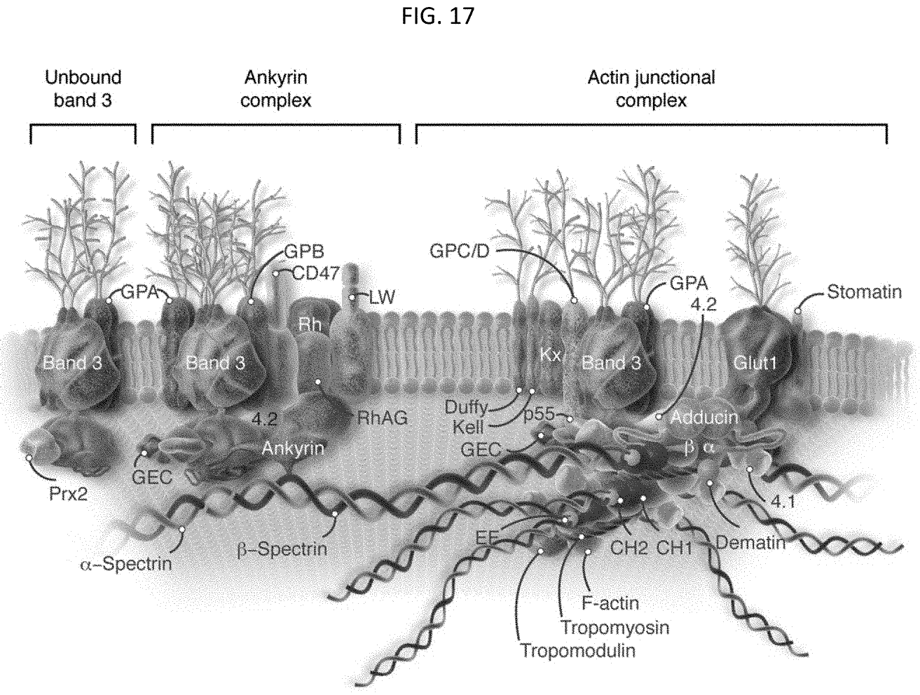

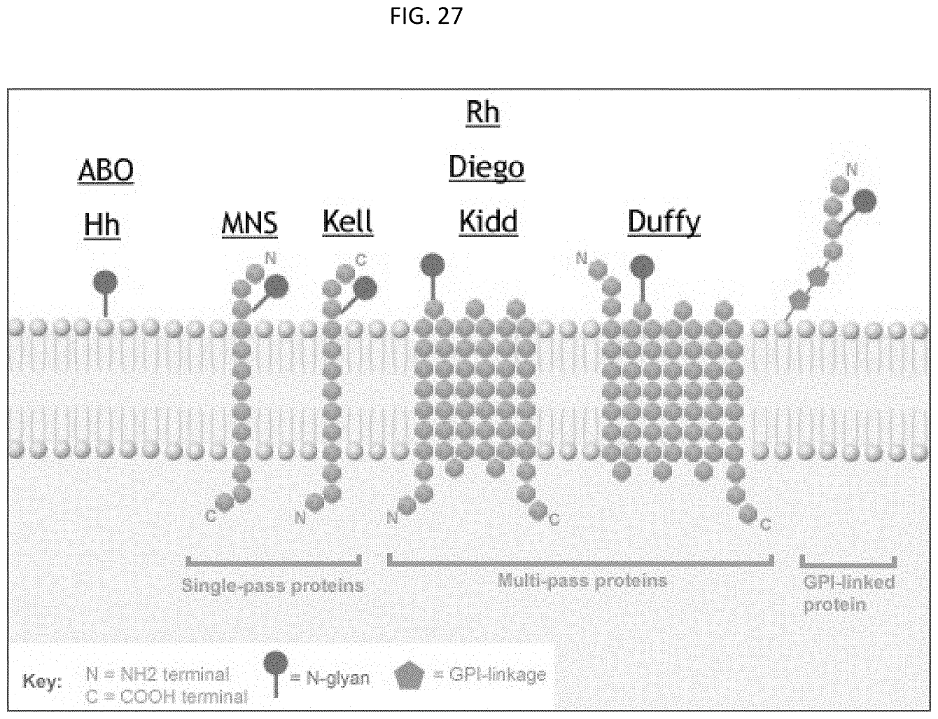

In general, a red blood cell membrane contains more than 300 antigens on its surface grouped into various blood group systems. A blood group system includes one or more antigens controlled at a single gene locus or by two or more very closely linked loci. Each system is genetically discrete from every other blood group system. See FIGS. 14, 16, 17, 27, and 28 for examples.

In antibody identification panels, labs test the most clinically relevant antigens of the major blood group systems, for example, those responsible for hemolytic transfusion reactions. High frequency or high prevalence antigens are the antigens that occur in greater than 99% of the population, for example, Rh17. Therefore, all antibody identification panels are screen for Rh17. If a patient has an antibody towards high prevalence antigen like Rh17, cells in the panel positive for Rh17 would give a positive hemagglutination reaction. Cells that do not express the Rh17 antigen should not agglutinate. Because Rh17 is the most common antigen, diagnostic labs require rare red blood cells with no Rh surface expression. These cell are exceedingly rare in the general population, with a frequency of less than 1 in 50,000 donors. Therefore, a need exists for RBCs expressing rare antigen phenotypes that is not dependent on donation by individuals.

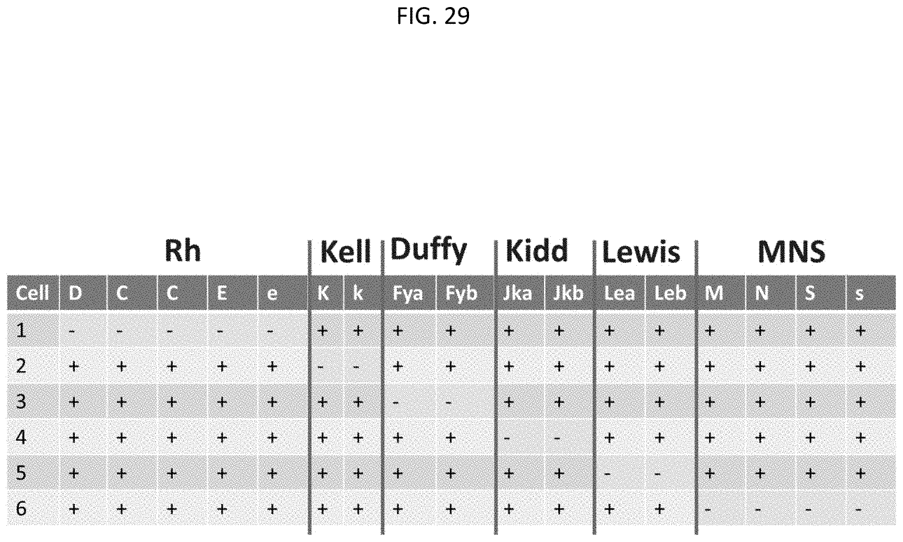

This embodiment describes antibody identification panels in which the red blood cells are engineered to remove/delete expression of an entire blood group system while simultaneously expressing all other blood group system antigens. (FIG. 29) For example, an RBC of the panel would not express any Rh antigen, but would express Kell, Duffy, Kidd, Lewis, MNS, etc. Addition of these designer red cells to the antibody identification panel will ease the antibody identification process.

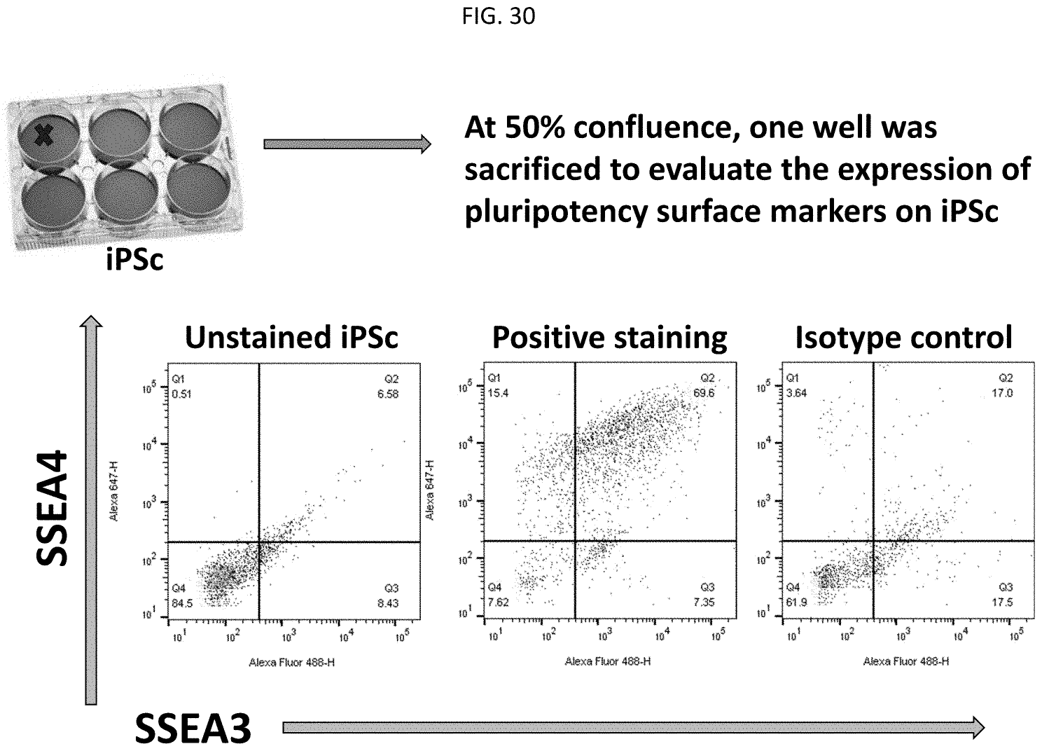

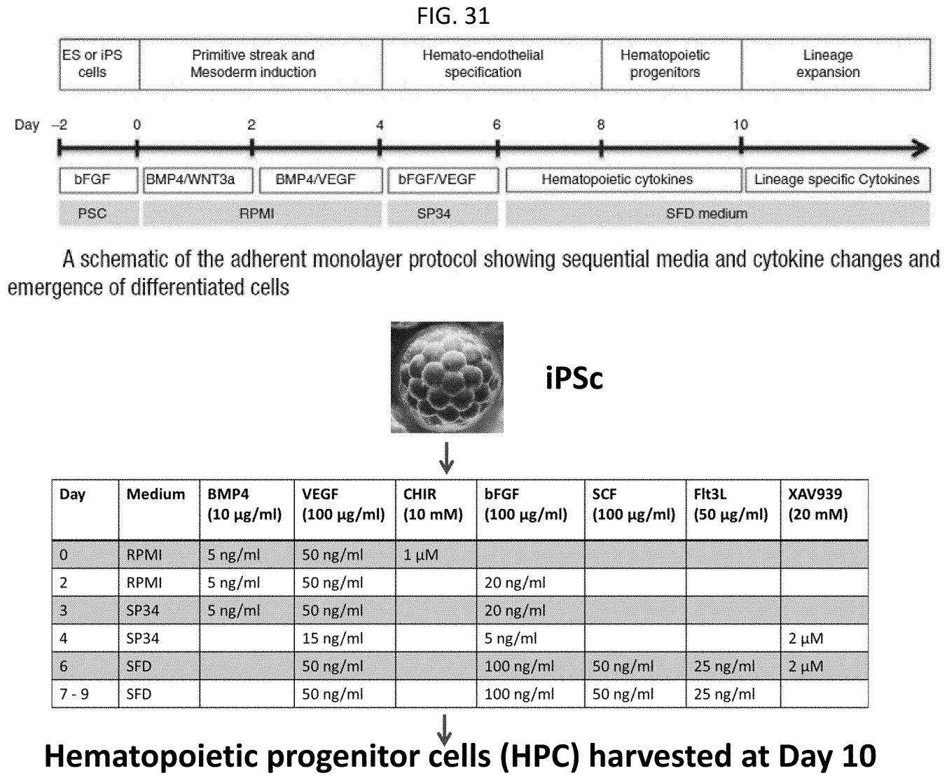

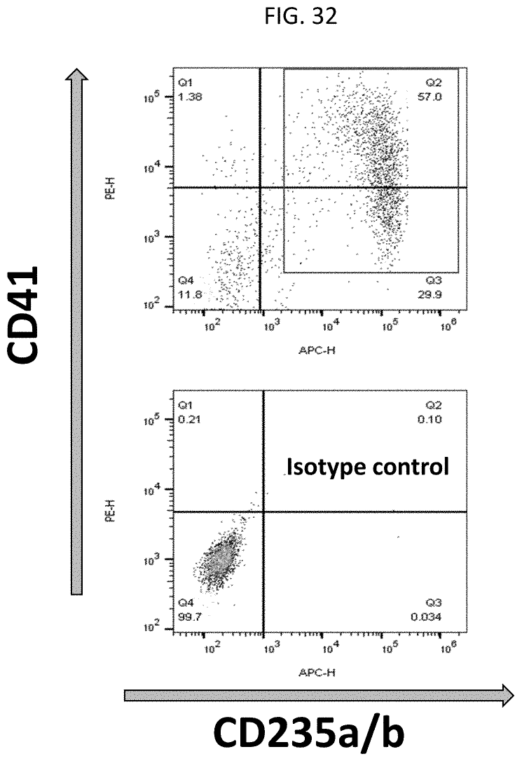

To produce designer red cells, induced pluripotent stem cells (iPS cells) may be genotyped for expression of major blood group system antigens. Expression of pluripotent surface markers on the iPS cells may also be evaluated. (FIG. 30). iPS cells are then differentiated into hematopoietic progenitor cells (HPCs). See, for example, Mills et al. (“Hematopoietic differentiation of pluripotent stem cells in culture,” Hematopoietic Stem Cell Protocols. Methods in Molecular Biology (Methods and Protocols), vol. 1185, Humana Press, New York, N.Y.). (FIG. 31). HPCs are identified based on CD41 and CD235a/b expression. (FIG. 32) Approximately 60-65% of multipotent HPC population were formed from differentiation of iPS cells. HPCs co-expressed CD41 (Integrin alpha chain 2b), a pan-hematopoietic marker and CD235a/b (Glycophorin A/B), a sialoglycoprotein typically found on erythrocyte membrane.

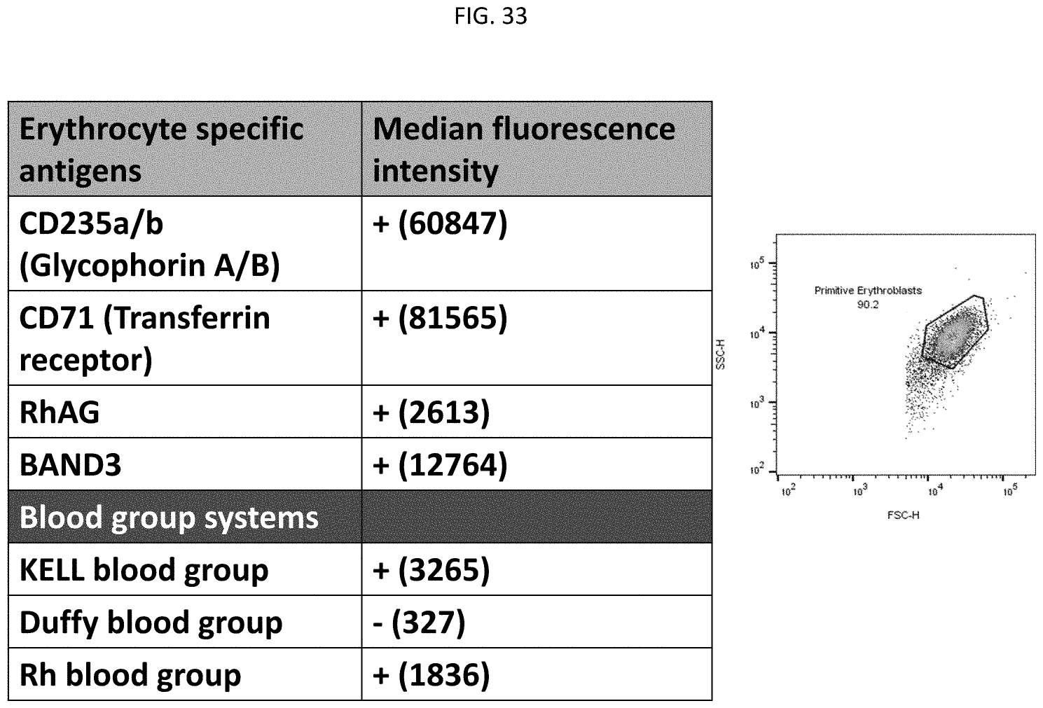

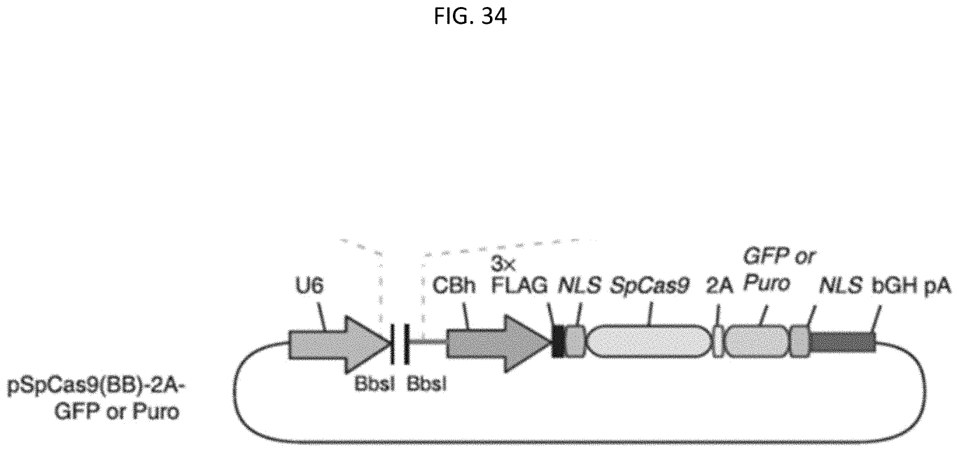

HPCs harvested at Day 10 were coated on ultra-low attachment polystyrene plates at 0.5×10⁶ cell/well, and cultured for 8 days (Days 10-17). Medium was changed every 2 days and at Day 17, erythroblasts were present. The culture medium was SFD medium with 50 ng/ml Stem Cell Factor (SCF) and 2 U EPO. Up to a 14 fold expansion in cell density was observed during differentiation of erythroblasts from HPCs. Erythroblasts were assayed for expression of various erythrocyte specific antigens and blood group system antigens. (FIG. 33)

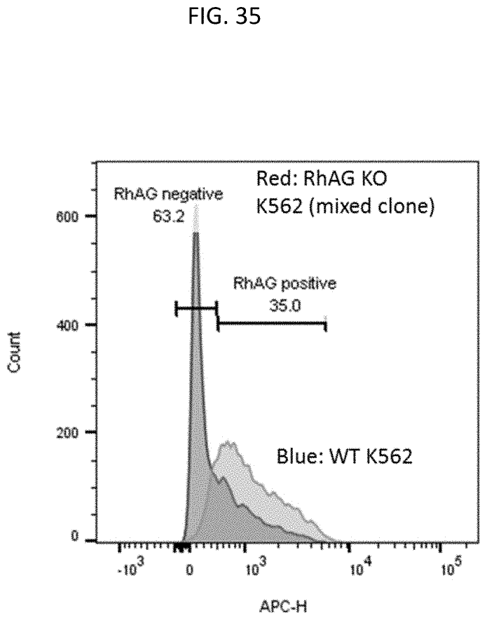

Preliminary engineering experiments were performed in K562 cells. K562 cells are a human erythroleukemia cell like that resembles proerythroblasts and express most of the red cell antigens. Guide RNA combinations were screened in K562 cells due to the ease of flow cytometry identification of positive gene editing events (FIG. 35).

Guide RNAs are designed and cloned into a suitable vector for transfection and expression into K562 cells of iPS cells. The vector included a GFP marker for selection of positive transformants. Gene deletion or mutation is then confirmed by sequencing and flow cytometry.

For transfection into iPS cells, the gRNA are cloned into the Cas9 pX459 expression vector, which includes a puromycin selectable marker. Positive transformants were selected based on puromycin resistance. Modification of target site was then confirmed by sequencing and PCR. iPS cells that include the desired modification are then differentiated into RBCs using the methods described previously.

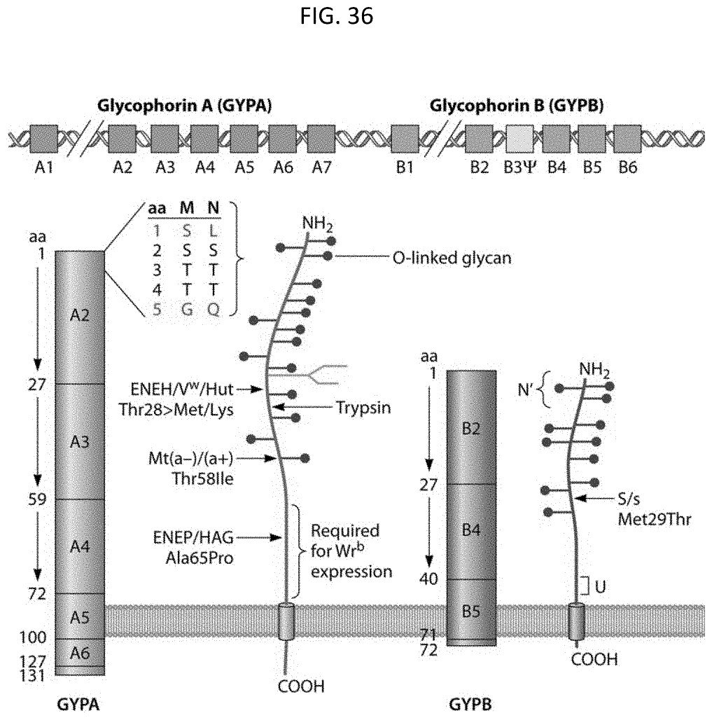

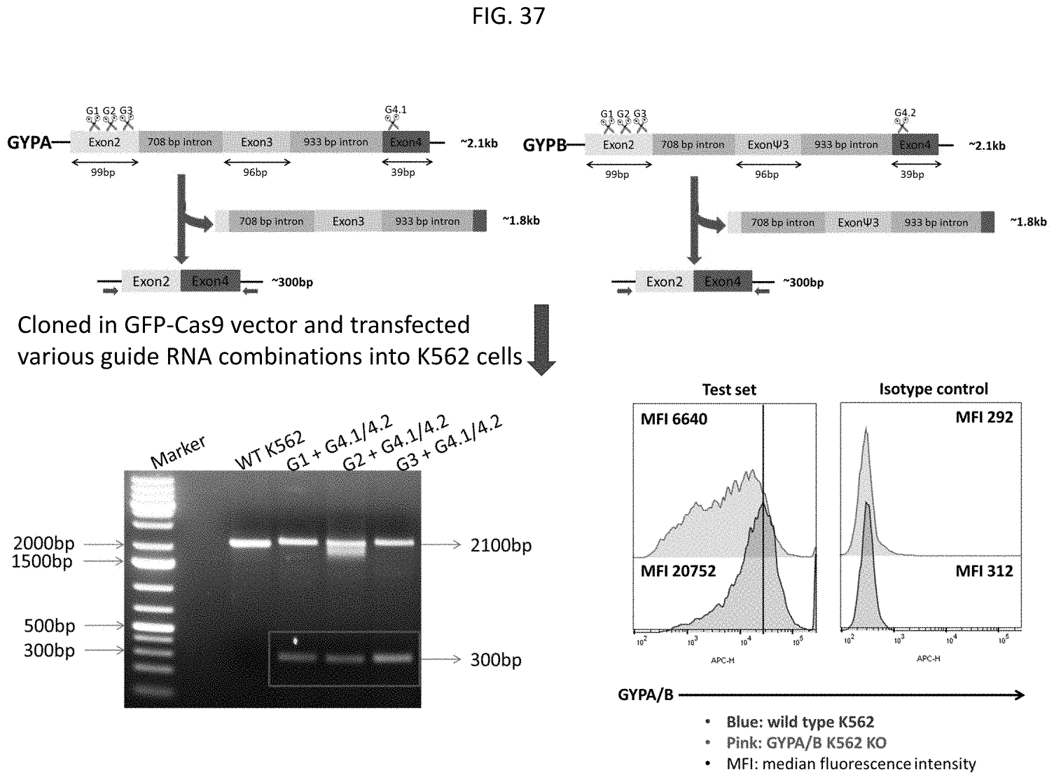

The same process is used to knock out the MNS blood group system by targeting the glycophorin A (GYPA) & B (GPYB) genes on chromosome 4. (FIGS. 36 and 37) These genes were formed by a gene duplication event and share 97% homology. Guide RNAs are designed to cut within exon 2 and exon 4 of each of GYPA and GYPB to remove all of exon 3 and the intervening introns (708 bp and 933 bp). iPS cells are transformed with the vector expressing the guide RNAs and Cas9 and iPS cells positive for the gene editing event are differentiated into RBCs using the methods described previously.

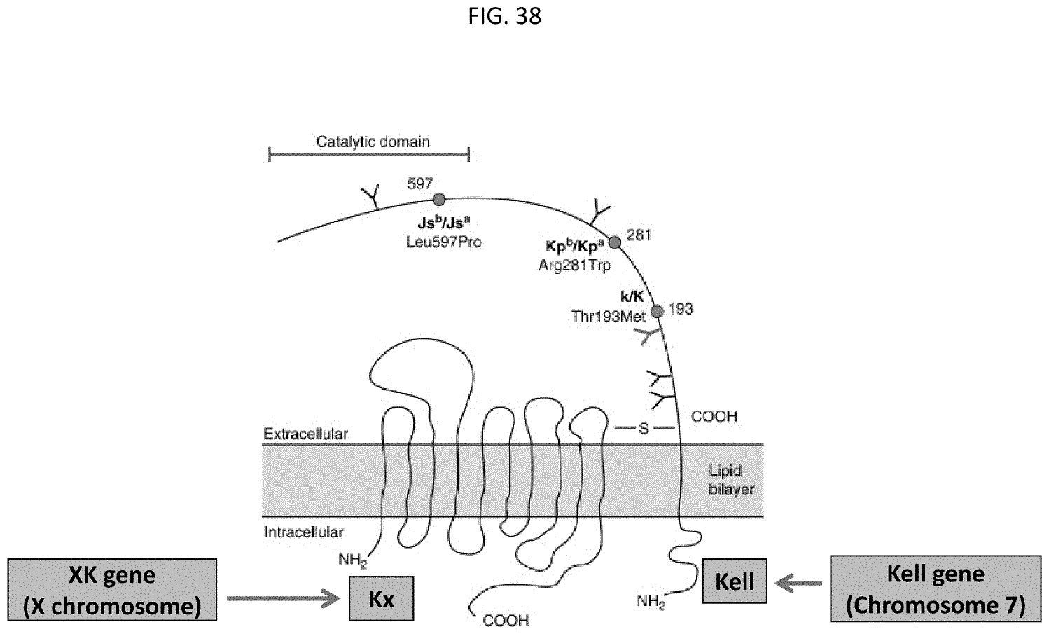

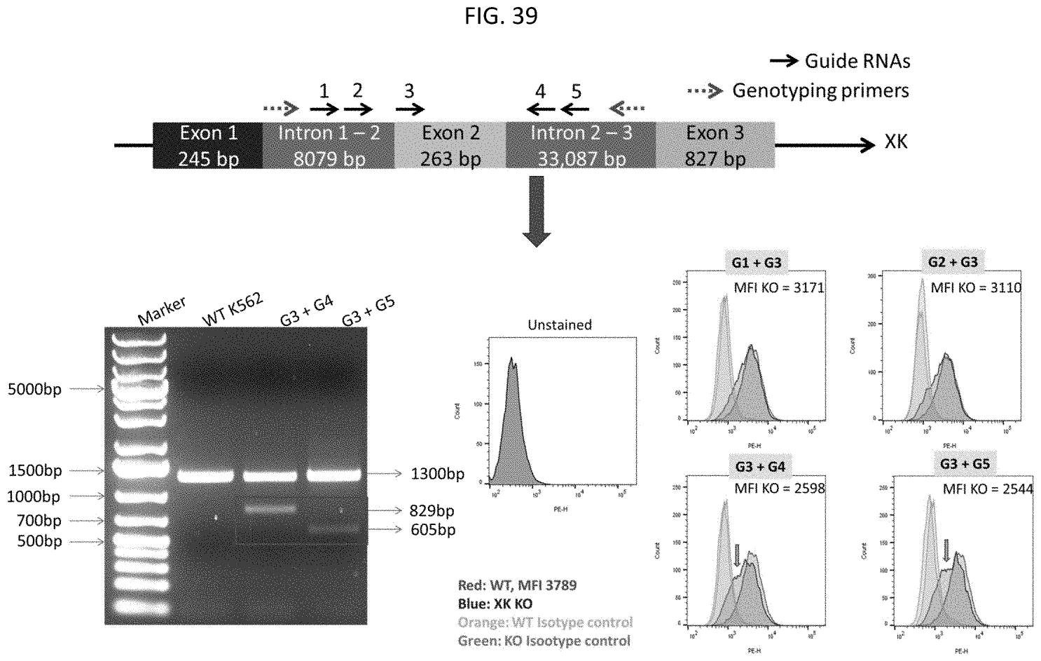

The process is also used to knock out the Kell blood group system by targeting the XK gene on the X chromosome. (FIGS. 38 and 39). Kell antigens are only very weakly expressed on XK negative cells, making the XK gene an appealing target for producing Kell blood group negative RBCs. Guide RNAs are designed to cut within intron 1 or exon 2 and intron 2 to remove all or a portion of exon 2. iPS cells are transformed with the vector expressing the guide RNAs and Cas9 and iPS cells positive for the gene editing event are differentiated into RBCs using the methods described previously.

We claim:

1. A method for creating a mammalian hematopoietic progenitor cell that does not express any Rh red blood cell antigen, the method comprising the steps of:
a) providing one or more guide RNAs designed to target a gene selected from the group consisting of RHD, RHCE, and RHAG;
b) ligating the guide RNA of step (a) into a plasmid encoding a Cas9 nuclease;
c) transfecting mammalian induced pluripotent stem cells with the plasmid of step (b);
d) cloning and selecting the resulting clones that do not express the Rh antigens; and
e) differentiating the selected clones into mammalian hematopoietic progenitor cells that do not express the Rh antigens.

2. The method of claim 1, wherein the target gene is RHD or RHCE.

3. The method of claim 1, wherein the induced pluripotent stem cell comprising the RHD, RHCE, and RNAG genes.

4. The method of claim 1, wherein the mammalian induced pluripotent stem cell is transfected with the plasmid of step (b) in the presence of a homology-directed repair (HDR) template oligonucleotide.

5. The method of claim 4, wherein the HDR template oligonucleotide encodes a stop codon to be introduced into the target gene.

6. The method of claim 4, wherein the HDR template oligonucleotide encodes missense mutation in the target gene.

7. The method of claim 4, wherein the HDR template oligonucleotide additionally encodes a diagnostic restriction enzyme site.

8. The method of claim 1, wherein the plasmid additionally encodes a reporter gene.

9. The method of claim 1, wherein the Cas9 nuclease is Cas9n.

10. A mammalian hematopoietic progenitor cell created by the method of claim 1.

11. A method for creating a mammalian hematopoietic progenitor cell that does not express any MNS red blood cell antigen, the method comprising the steps of:
a) providing one or more guide RNAs designed to target a genes GYPA and GYPB;
b) ligating the guide RNA of step (a) into a plasmid encoding a Cas9 nuclease;
c) transfecting mammalian induced pluripotent stem cells with the plasmid of step (b);
d) cloning and selecting the resulting clones that do not express the MNS antigens; and
e) differentiating the selected clones into mammalian hematopoietic progenitor cells that do not express the MNS antigens.

12. The method of claim 11, wherein the induced pluripotent stem cell comprises the GYPA and GYPB genes.

13. The method of claim 11, wherein the mammalian induced pluripotent stem cell is transfected with the plasmid of step (b) in the presence of a homology-directed repair (HDR) template oligonucleotide.

14. The method of claim 14, wherein the HDR template oligonucleotide encodes a stop codon to be introduced into the target genes.

15. The method of claim 14, wherein the HDR template oligonucleotide encodes missense mutation in the target genes.

16. The method of claim 14, wherein the HDR template oligonucleotide additionally encodes a diagnostic restriction enzyme site.

17. The method of claim 11, wherein the plasmid additionally encodes a reporter gene.

18. The method of claim 11, wherein the Cas9 nuclease is Cas9n.

19. A mammalian hematopoietic progenitor cell created by the method of claim 11.

20. A method for creating a mammalian hematopoietic progenitor cell that does not express any Kell red blood cell antigen, the method comprising the steps of:
a) providing one or more guide RNAs designed to target a genes selected from the group consisting of XK and KEL;
b) ligating the guide RNA of step (a) into a plasmid encoding a Cas9 nuclease;
c) transfecting mammalian induced pluripotent stem cells with the plasmid of step (b);
d) cloning and selecting the resulting clones that do not express the Kell antigens; and
e) differentiating the selected clones into mammalian hematopoietic progenitor cells that do not express the Kell antigens.

21. A method for creating mammalian cells that does not express any Rh red blood cell antigen, the method comprising the steps of:
a) providing one or more guide RNAs designed to target a gene selected from the group consisting of RHD, RHCE, and RHAG;
b) ligating the guide RNA of step (a) into a plasmid encoding a Cas9 nuclease;
c) transfecting the mammalian cell with the plasmid of step (b), wherein the mammalian cell is selected from the group consisting of a mammalian pluripotent stem cell, a K562 erythro-leukemia cell, or a DAMI cell;
d) cloning and selecting the resulting clones that do not express the Rh antigens; and
e) expanding the selected clones in culture to produce mammalian cells that do not express the Rh antigens.

22. A method for creating mammalian cells expressing a specific platelet alloantigen, the method comprising the steps of:
a) providing one or more guide RNAs designed to target platelet alloantigen target locus;
b) ligating the guide RNA of step (a) into a plasmid encoding a Cas9 nuclease;
c) transfecting the mammalian cell with the plasmid of step (b) in the presence of a homology directed repair template oligonucleotide, wherein the mammalian cell is selected from the group consisting of a mammalian pluripotent stem cell, a K562 erythro-leukemia cell, or a DAMI cell;
d) cloning and selecting the resulting clones that do not express the platelet alloantigen of interest; and
e) expanding the selected clones in culture to produce mammalian cells that express the platelet alloantigen of interest.
